(19) United States
(12) Patent Application Publication
(10) Pub. No.: US 2020/0095589 A1
(43) Pub. Date: Mar. 26, 2020

(54) ENGINEERED DCAS9 WITH REDUCED TOXICITY AND ITS USE IN GENETIC CIRCUITS

(71) Applicant: Massachusetts Institute of Technology, Cambridge, MA (US)

(72) Inventors: Christopher A. Voigt, Belmont, MA (US); Shuyi Zhang, Boston, MA (US)

(73) Assignee: Massachusetts Institute of Technology, Cambridge, MA (US)

(21) Appl. No.: 16/581,918

(22) Filed: Sep. 25, 2019

Related U.S. Application Data
(60) Provisional application No. 62/735,877, filed on Sep. 25, 2018.

(51) Int. Cl. C12N 15/62; C12N 9/22; C12N 9/02; C07K 14/37

(52) U.S. Cl. CPC C12N 15/62; C12N 9/22; C12N 9/0071; C12Y 114/17003; C07K 2319/00; C07K 14/37; C12N 2310/20; C12N 2800/80; C12Y 301/22004

(57) ABSTRACT

Disclosed herein are novel CRISPR/dCas9-based fusion proteins that facilitate the scaling up of genetic circuits, including those with non-linear response curves. The dCas9 based fusion proteins produce significantly less toxicity in comparison to previously described CRISPR/Cas9-based proteins used with logic gates. These improvements enable the generation of complex genetic circuits when both digital response curves and large amounts of dCas9 protein are needed. Also disclosed herein are methods of regulating expression of an output sequence through the introduction of novel CRISPR/dCas-9-based fusion proteins and genetic circuits into a cell.

### RELATED APPLICATION

This application claims priority under 35 U.S.C. § 119(e) to U.S. patent application No. 62/735,877, filed Sep. 25, 2018, the entire contents of which are incorporated herein by reference.

### GOVERNMENT SUPPORT

This invention was made with Government support under Grant No. N00014-16-1-2388 awarded by the Office of Naval Research. The Government has certain rights in the invention.

### INCORPORATION BY REFERENCE

This application contains a Sequence Listing which has been filed electronically in ASCII and is hereby incorporated by reference in its entirety. This ASCII copy, created on Dec. 2, 2019, is named M065670412US01-SUBSEQ-CRP and is 98.199 kB in size.

### FIELD

Disclosed herein are novel CRISPR/dCas9-based fusion proteins that produce significantly less toxicity in comparison to previously described CRISPR/Cas9-based proteins, and complex genetic circuits controlled by the novel CRISPR/dCas-9-based fusion proteins.

### BACKGROUND

Synthetic regulatory networks enable the control of when genes are turned on (Khalil A. S. and Collins J. J., Nat. Rev. Genet., 2010 May; 11(5): 367-79). Natural networks can consist of hundreds of regulators, but implementing synthetic versions at this scale has proven elusive (Purnick P. E. and Weiss R., Nat. Rev. Mol. Cell. Biol., 2009 June; 10(6): 410-22). Regulators used to build such networks have to perform reliably, cannot interfere with each other, and must tax cellular resources minimally (Nielsen A. A., et al., Curr. Opin. Chem. Biol., 2013 December; 17(6): 878-92). Sets of protein-based repressors and activators have been used to build regulatory circuits, but expanding the set becomes increasingly difficult as each new protein needs to be tested for cross-reactions with the remainder in the set (Gaber R., et al., Nat. Chem. Biol., 2014 March; 10(3): 203-8; Garg A., et al., Nucleic Acids Res., 2012 August; 40(15): 7584-95; Li Y., et al., Nat. Chem. Biol., 2015 March; 11(3): 207-13; Nielsen A. A., et al., Science, 2016. 352(6281): aac7341; Stanton B. C., et al., Nat. Chem. Biol., 2014. 10(2): p. 99-105). Further, protein expression draws on cellular resources (ATP, ribosomes, amino acids, etc.), and this can result in slow growth, reduced metabolic performance, and evolutionary instability (Ceroni F., et al., Nat. Methods, 2018 May; 15(5): 387-93; Lynch M. and Marinov G. K., Proc. Natl. Acad. Sci. USA, 2015 Dec. 22; 112(51): 15690-5; Pasini M., et al., N. Biotechnol. 2016 Jan. 25; 33(1): 78-90).

Regulators based on CRISPR (clustered regularly interspaced short palindromic repeats) machinery offer a potential solution (Barrangou R., et al., Science, 2007 Mar. 23; 315(5819): 1709-12; Deltcheva E., et al., Nature, 2011 Mar. 31; 471(7340): 602-7; Jinek M., et al., Science, 2012. 337(6096): p. 816-821; Cong L., et al., Science, 2013. 339(6121): p. 819-23; Mali P., et al., Science, 2013. 339(6121): p. 823-26; Gasiunas G., et al., Proc. Natl. Acad. Sci. USA, 2012 Sep. 25; 109(39): 15539-40). Catalytically inactive dCas9 can be used as a repressor by using the small guide RNA (sgRNA) to target a sequence within a promoter to sterically block RNA polymerase (RNAP) (Qi Lei S., et al., Cell, 2013. 152(5): p. 1173-83; Bikard D., et al., Nucleic Acids Res., 2013 August; 41(15): 7429-37). The target sequence in the promoter is based on a 3 nt PAM sequence, which binds to the dCas9 protein, and a 20 nt targeting region that basepairs with the sgRNA. Different DNA sequences can be targeted by changing this region, which has been the basis for building large sets of sgRNA-promoter pairs that exhibit little or no crosstalk. Up to 5 pairs have been shown in E. coli (Nielsen A. A. and Voigt C. A., Mol. Syst. Biol., 2014. 10(763): 1-11) and up to 20 pairs in yeast (Gander M. W., et al., Nat. Commun., 2017 May 25; 8: 15459), but theoretically thousands could be made, essentially solving the need for orthogonal regulators to build large networks. In addition, sgRNA-circuits do not require translation to function, thus simplifying their use in the nucleus of eukaryotic cells. Previously, dCas9 has been used to build simple logic circuits and cascades with up to 3 sgRNAs in bacteria, 7 sgRNAs in yeast, and 4 sgRNAs in mammalian cells (Nielsen A. A. and Voigt C. A., Mol. Syst. Biol., 2014. 10(763): 1-11; Gander M. W., et al., Nat. Commun., 2017 May 25; 8: 15459; Didovyk A., et al., ACS Synth. Biol., 2016 Jan. 15; 5(1): 81-8; Gao Y., et al., Nat. Methods, 2016 December; 13(12): 1043-49; Holowko M. B., et al., ACS Synth. Biol., 2016 Nov. 18; 5(11): 1275-83; Kiani S., et al., Nat. Methods, 2014 July; 11(7): 723-6; Weinberg B. H., P et al., Nat. Biotechnol., 2017 May; 35(5): 453-62).

Despite the promise, there are several limitations in the scale-up of dCas9-based circuits. The foremost challenge is that high concentrations of dCas9 is toxic in many bacteria (Rock J. M., et al., Nat. Microbiol., 2017. 2(16274): p. 1-9; Cho S., et al., ACS Synth. Biol., 2018 Apr. 20; 7(4): 1085-94; Lee Y. J., et al., Nucleic Acids Res., 2016 Mar. 18; 44(5): 2462-73). This can be avoided for genome editing and CRISPR interference (CRISPRi) experiments by keeping the concentration low or limiting how long it is expressed (Peters J. M., et al., Curr. Opin. Microbiol., 2015 October; 27: 121-26). However, for a genetic circuit, dCas9 needs to be continuously available, including under the conditions required by the application, for example in a fermenter. This is compounded by the problem that multiple sgRNAs all have to share the same pool of dCas9. The draw-down of a shared resource leads to changes in performance of all the sgRNA, referred to as “retroactivity,” and this can have a damaging impact on circuit function (Del Vecchio D., et al., Mol. Syst. Biol., 2008. 4(161): 1-16; Jayanthi S., et al., ACS Synth. Biol., 2-13 Aug. 16; 2(8): 431-41; Brewster R. C., et al., Cell, 2014 March; 156(6): 1312-23; Qian Y., et al., ACS Synth. Biol., 2017 Jul. 21; 6(7): 1263-72). Further, sgRNA-based gates have remarkably low cooperativity (Hill coefficient n≈1.0) (Nielsen A. A. and Voigt C. A., Mol. Syst. Biol., 2014. 10(763): 1-11). Higher cooperativities are required to build regulation that implement multistable switches, feedback control, cascades, and oscillations (n>1) (Strogatz S. H., Hachette UK, 2014; Hooshangi S., et al., Proc. Natl. Acad. Sci. USA, 2005 Mar. 8; 102(10): 3581-86; Ferrell J. E. Jr and Ha S. H., Trends Biochem. Sci., 2014 December; 39(12): 612-8; Gardner T. S., et al., Nature, 2000 Jan. 20; 403(6767): 339-42). In yeast, the cooperativity of sgRNA-based regulation was increased by fusing dCas9 to the chromatin remodeling repression domain Mxil, but there is no equivalent approach for prokaryotes (Gander M. W., et al., Nat. Commun., 2017 May 25; 8: 15459).

### SUMMARY

The origins of dCas9 toxicity are poorly understood. It has been observed that dCas9 binds non-specifically to NGG PAM sites, particularly when unbound to a sgRNA, and there are many GG sequences in the genome (5.4×10⁵ PAM sites per E. coli genome) (Jones D. L., et al., Science, 2017 Sep. 29; 357(6358): 1420-24). While it primarily binds to this motif, it has been shown that it can also inefficiently recognize other PAM sequences (e.g., NAG or NGA) (Hsu P. D., et al., Nat. Biotechnol., 2013. 31(9): 827-32; Zhang Y., et al., Sci. Rep., 2014. 4(5405): 1-5). Further, dCas9 functions by first actively interrogating the genome to search for the PAM motif, and then checking the complementarity of the sgRNA sequence to the target site (Jinek M., et al., Science, 2012. 337(6096): 816-821; Qi Lei S., et al., Cell, 2013. 152(5): 1173-83). The search for PAM binding involves actively opening the DNA double strands in the chromosome (Sternberg S. H., et al., Nature, 2014. 507(7490): 62-67). Previous studies also demonstrated that off-target genomic loci with up to six nucleotides that differ from the sgRNA sequence could still be recognized by Cas9, albeit with lower efficiency (but still requiring the PAM site) (Kim D., et al., Nat. Methods, 2015. 12(3): 237-43). These observations collectively point to the non-specific binding to NGG sequences by dCas9 as being a significant contributor to toxicity.

It was hypothesized that reducing the non-specific binding of dCas9 would alleviate toxicity. The specificity of active Cas9 for genome editing applications has been increased via a variety of strategies, including point mutations to enhance PAM binding (Kleinstiver B. P., et al., Nature, 2015. 523(7561): p. 481-85; Slaymaker I. M., et al., Science, 2016. 351(6268): 84-88), increasing sgRNA length (Fu Y., et al., Nat. Biotechnol., 2014. 32(3): 279-84; Chen B., et al., Cell, 2013. 155(7): 1479-91), splitting Cas9 (Zetsche B., et al., Nat. Biotechnol., 2015. 33(2): 139-42; Nihongaki Y., et al., Nat. Biotechnol., 2015. 33(7): 755-60; Wright A. V., et al., Proc. Natl. Acad. Sci. USA, 2015. 112(10): 2984-89), and the use of a pair of Cas9 nickases or FokI-dCas9 nucleases to increase the length of targeting sequence (Mali P., et al., Nat. Biotechnol., 2013. 31(9): p. 833-38; Guilinger J. P., et al., Nat. Biotechnol., 2014. 32(6): 577-82). It has been shown that Cas9 can be mutated (R1335K) to impair its ability to recognize the PAM, thus completely blocking DNA cleavage (Bolukbasi M. F., et al., Nat. Methods, 2015 December; 12(12): 1150-56). Cleavage could be partially rescued by fusing a DNA binding protein (a ZFP or TALE) to dCas9 and placing the corresponding operator upstream of the region targeted by the sgRNA. The longer effective “operator” increase cleavages specificity.

As described herein, this strategy was applied to dCas9, but it was found that a fusion to the TetR-family PhlF repressor is uniquely able to recover full activity. This essentially eliminated toxicity, thus allowing up to 9600 proteins per cell without impairing cell health. Promoters were constructed that include the 30 bp PhlF operator and the sgRNA targeting sequence. A set of 30 sgRNAs were constructed and characterized as NOT gates with improved cooperativity (<n>=1.6). Finally, the loss in dynamic range of a gate as additional sgRNAs are expressed was quantified and a mathematical model was used to quantify the loss in repression due to resource sharing. This disclosure represents the first step towards harnessing dCas9 to scale-up circuit design; however, it also exposes limitations in the use of many regulators that require a shared pool of proteins for activity.

Described herein are novel CRISPR/dCas9-based logic gates that facilitate the scaling up of genetic circuits. These logic gates exhibit non-linear response curves and significantly less toxicity in comparison to previously described CRISPR/Cas9-based logic gates. These improvements enable the production of complex genetic circuits when both digital response curves and large amounts of dCas9 protein are needed. Also described herein are methods of regulating expression of a genetic circuit output sequence through the introduction of novel CRISPR/dCas9-based logic gates into a cell.

Compositions of Synthetic Genetic Circuits and Non-Natural Cells

In one aspect, the components of a synthetic genetic circuit are provided, including a single polynucleotide or a combination of polynucleotides that encode: at least one fusion protein comprising a catalytically-inactive CRISPR/Cas protein fused to a transcription factor, wherein the catalytically-inactive CRISPR/Cas protein comprises a mutated protospacer adjacent motif (PAM) domain (or PAM-interacting domain) and a mutated or absent HNH domain, at least one small guide RNA, and at least one output sequence whose expression is operably linked to an output promoter, wherein the output promoter comprises a transcription factor operator and a cognate promoter comprising an sgRNA target site and, optionally, a PAM site.

In some embodiments, the mutation of the CRISPR/Cas HNH domain consists of the deletion of the entire domain and its replacement by an amino acid linker sequence (e.g., GGSGGS, SEQ ID NO: 127). In some embodiments, the catalytically-inactive CRISPR/Cas protein of a fusion protein possesses a functional RuvC domain. In some embodiments, the catalytically-inactive CRISPR/Cas protein of a fusion protein consists of amino acids 1 to 1368 of Cas9, wherein the Cas9 amino acid sequence contains D10A and R1335K mutations and the Cas9 amino acids 768 to 919 are replaced by a GGSGGS (SEQ ID NO: 127) amino acid linker sequence.

In some embodiments, the catalytically-inactive CRISPR/Cas protein is fused to the transcription factor with a C-terminal polypeptide bond. In other embodiments, the catalytically-inactive CRISPR/Cas protein is fused to the transcription factor with an N-terminal polypeptide bond. In some embodiments, the catalytically-inactive CRISPR/Cas protein and the transcription factor are separated by a linker peptide.

In some embodiments, the transcription factor of a fusion protein represses (or decreases) the expression of the output sequence. In some embodiments, the transcription factor of a fusion protein is PhlF or an ortholog or functional variant, thereof. In other embodiments, the transcription factor of a fusion protein is BM3RI or an ortholog or functional variant, thereof. In other embodiments, the transcription factor of a fusion protein is a ZFP protein or an ortholog or functional variant, thereof. In some embodiments, the transcription factor of a fusion protein activates (or increases) the expression of the output sequence.

In some embodiments, the transcription factor operator and the cognate promoter of the output promoter are on the same DNA strand. In other embodiments, the transcription factor operator and the cognate promoter of the output promoter are on complementary DNA strands. In some embodiments, the transcription factor operator and the cognate promoter of the output promoter are separated by 0 to 20 base pairs.

In some embodiments, the catalytically-inactive CRISPR/Cas protein consists of amino acids 1 to 1368 of Cas9, wherein the Cas9 amino acid sequence contains D10A and R1335K mutations and the Cas9 amino acids 768 to 919 are replaced by a GGSGGS (SEQ ID NO: 127) amino acid linker sequence, the transcription factor is PhlF, the catalytically-inactive CRISPR/Cas protein is fused to PhlF with a C-terminal polypeptide bond, the transcription factor operator of the output promoter is a PhlF operator, and the PhlF operator and the cognate promoter sequence of the output promoter are separated by 0 to 20 base pairs.

In some embodiments, the single polynucleotide or the combination of polynucleotides of a genetic circuit encode: (a) at least one fusion protein comprising a catalytically-inactive CRISPR/Cas protein fused to a transcription factor, wherein the catalytically-inactive CRISPR/Cas protein comprises a mutated PAM domain and a mutated or absent HNH domain; (b) between two and thirty unique sgRNAs, wherein the expression of at least one of the unique sgRNAs is under the control of an inducible promoter; and (c) between one and twenty-nine output sequences, each of whose expression is operably linked to an independent output promoter, wherein at least two of the output promoters comprise a transcription factor operator and a cognate promoter comprising a unique sgRNA target site and, optionally, a PAM site, and wherein: (i) the unique sgRNA target site of each output promoter comprising an sgRNA target site comprises an sgRNA target site of one of the sgRNAs in (b); and (ii) the unique sgRNA target site of at least one of the output promoters comprises the sgRNA target site of the at least one sgRNA under the control of an inducible promoter in (b).

In some embodiments, the genetic circuit is encoded on a single polynucleotide. In some embodiments, the single polynucleotide is a plasmid. In some embodiments, the genetic circuit is encoded on more than one polynucleotides. In some embodiments, at least one of the more than one polynucleotides is a plasmid.

In another aspect, a polynucleotide or combination of polynucleotides are provided. In some embodiments, the polynucleotide or combination of polynucleotides comprise(s) the nucleotide sequence of a genetic circuit described above. Also disclosed herein are compositions comprising the polynucleotide or combination of polynucleotides.

In another aspect, the disclosure relates to non-natural cells comprising a genetic circuit as described above or a polynucleotide or combination of polynucleotides as described above.

Compositions of Fusion Proteins

In another aspect, compositions of fusion proteins are provided, including a catalytically-inactive Cas9 protein linked by a C-terminal polypeptide bond to PhlF, wherein the catalytically-inactive Cas9 protein comprises a mutated PAM domain, a mutated HNH domain, and a functional RuvCI domain, and optionally, the catalytically-inactive Cas9 protein and the PhlF protein are separated by a linker peptide.

In some embodiments, the mutation of the Cas9 HNH domain consists of the deletion of the entire domain and its replacement by an amino acid linker sequence. In some embodiments, the catalytically-inactive Cas9 protein amino acid sequence contains D10A and R1335K mutations and the Cas9 amino acids 768 to 919 are replaced by a GGSGGS (SEQ ID NO: 127) amino acid linker sequence.

Composition of Polynucleotides

In another aspect, compositions of polynucleotides encoding for fusion proteins are provided, including compositions of one or more polynucleotides encoding for any fusion protein encompassed above in “Compositions of Fusion Proteins.”

Methods of Regulating Expression of a Genetic Circuit's Output Sequence

In another aspect, methods of regulating expression of a genetic circuit's output sequence are described, including the introduction of a synthetic genetic circuit into a cell. This aspect embodies the cellular introduction of the synthetic genetic circuit compositions encompassed above in “Components of a Synthetic Circuit.”

These and other aspects are descried in more detail below.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1G. Design and evaluation of a dCas9—transcription factor fusion. FIG. 1A. A schematic of targeted repression by dCas9-sgRNA complex bound to the promoter region of a fluorescent reporter gene (RFP, red fluorescent protein). FIG. 1B. A schematic of the fused protein bound to a promoter. DBD is the DNA-binding domain that is fused to dCas9. GGN is the PAM site. R133fK is the mutation that reduces the PAM recognition abilty of dCas9. FIG. 1C. The impact of changes to the fused protein and promoter on the response. The fold-repression is calculated as the ratio of uninduced to induced (1 mM IPTG) cells (Methods). All constructs other than the first are based on dCas9*(R1335K). F and R represent the forward and reverse orientations of the Zif268 operator. AHNH refers to the deletion of this domain. L88 shows the impact of a longer linker. The size of the spacer between the −35 and operator sequence is shown as SN, where N is the number of bp. Sequences and plasmid maps are shown in FIGS. 15A-15F and TABLE 3. SrpR, HlyIIR and BM3RI are all TetR-family repressors that were tested as alternatives to PhlF. FIG. 1D. The growth impact of dCas9 and dCas9*_PhlF is compared to the pSZ_Backbone plasmid (FIGS. 15A-15F) as a control. Protein expression is controlled using the aTc-inducible system and the x-axis is shown in units of fluorescence for the pTet promoter, measured separately (FIG. 4). The dashed line shows 2.5 ng/ml aTc, used in FIG. 1E for morphology studies. The arrows point to the inducer levels (0.7 ng/ml and 2.5 ng/ml) where the protein concentrations are determined in FIG. 1G. Media and growth conditions are provided in the Methods. FIG. 1E. Microscopic images of E. coli strains expressing PhlF, dCas9 or dCas9*_PhlF variants and a control (Backbone) are shown, under identical conditions as used for the growth curves. The scale bars are 5 μm. The corresponding FSC-A/SSC-A distribution of each strain was measured by flow cytometry (Methods). FIG. 1F. The fold-repression of the construct (pSZ_PhlF plasmid in FIGS. 13A-13F and the pPhlF_S6 promoter from TABLE 3) is shown as a function of dCas9*_PhlF expression. The sgRNA is under the control of the pTac promoter and all data are for 1 mM IPTG. The x-axis is the same as described in FIG. 1D. The line shows a fit to a Hill equation. For FIGS. 1B-1F, the data are shown as the mean of three experiments performed on different days and the error bars are the standard deviation. FIG. 1G. A representative immunoblotting assay is shown for calculating the number of dCas9 per cell. The dashed lines show the interpolation used to estimate concentrations. The calculation is described in the Methods and the numbers presented in the text are based on three experiments performed on different days (FIGS. 6A-6C).

FIGS. 2A-2D. NOT gates based on dCas9*_PhlF. FIG. 2A. The schematic of the gate is shown. The input and output to the gate are pTac and p9. Part sequences and plasmid maps are provided in FIGS. 15A-15F and TABLE 4. FIG. 2B. The response curves of dCas9-based NOT gates are shown (Methods). The input is the activity of the pTac promoter as a function of IPTG concentration, measured separately (FIG. 4). The concentration of dCas9*_PhlF was maintained by adding 2.5 ng/ml aTc and 0.7 ng/ml for dCas9. FIG. 2C. The response functions of 30 NOT gates based on orthogonal pairs of sgRNAs and promoters. The sequences are provided in FIG. 14. The data were fit to Equation 1 of Example 4 and the resulting parameters are provided in TABLE 1. FIG. 2D. Evaluation of cascades of different length. The detailed parts used in the genetic systems are shown in FIGS. 16A-16D. The input to the gate is the vanillic acid inducible promoter (pVan) and the x-axis is the activity of this promoter at different levels of inducer, measured separately (FIG. 4). The fits to the data are the responses predicted by combining the response functions of each layer of the cascade. The response functions of the individual gates and the predicted propagation of the signal through the cascade are shown at the bottom (Methods). All of the data in this Figure are shown as the mean of three experiments performed on different days and the error bars are the standard deviation.

FIGS. 3A-3B. The impact of simultaneous expression of multiple sgRNAs. FIG. 3A. Expression of sgRNA9 was fully induced (10 mM choline) to measure fold-repression of promoter p9 (labeled with asterisk), while the expression level of sgRNA10 (labeled with triangle) was induced by adding different levels of vanillic acid. The activity of the pVan promoter was measured separately as a function of vanillic acid concentration (FIG. 4). The detailed parts used in the genetic systems are shown in FIGS. 16A-16D. Solid lines are model prediction results. FIG. 3B. The impact of expressing multiple sgRNAs simultaneously. The repression fold change of promoter p9 was measured with or without the addition 100 μM vanillic acid. The constructs containing different numbers of sgRNAs are shown to the right. The sequences corresponding to the promoters and terminators are provided in TABLE 4. The sgRNAs are labeled sgN where N corresponds to the sequences in FIG. 14. The horizontal line marks 10-fold repression, roughly the minimum required for useful NOT gates. For dCas9*_PhlF, the fit parameters for equation 11 and 12 are β=3.0×10⁻¹¹ Ms⁻¹, α₁=7.6×10⁻¹² Ms⁻¹, αₓ=2.3×10⁻¹¹ Ms⁻¹, K=1.7×10⁻⁸M, n=0.9. For dCas9, the fit parameters are: β=3.0×10⁻¹¹ Ms⁻¹, α₁=7.6×10⁻¹² Ms⁻¹, αₓ=2.3×10⁻¹¹ Ms⁻¹, K=2.9×10⁻⁹ M, n=1.1. In both parts, the data are shown as the mean of three experiments performed on different days and the error bars are the standard deviation.

FIG. 4. Response curves of inducible systems. From left to right: pSZ_pTet, pSZ_Input, pSZ_Sensor (FIGS. 15A-15F and FIGS. 16A-16D). The solid line in each figure is a fit to a Hill equation. The pTet promoter activities were used to compare the expression levels of dCas9 in FIGS. 1D and 1F. The average of three experiments performed on different days is shown and the error bars indicate the standard deviation.

FIG. 5. Numbers of cells per ml as a function of optical density (OD₆₀₀). These data are used to calculate protein concentrations. After growth, aliquots were diluted 2×10⁷-fold and plated on LB agar (Methods). The colony numbers were then counted after overnight growth at 37° C. A linear regression curve (y=8.7×10⁸×) was fit to these data and used to calculate protein numbers per cell. The average of three experiments performed on different days is shown and the error bars indicate the standard deviation.

FIGS. 6A-6C. Immunoblotting and protein number estimation. For each figure: The concentration of Cas9 standard (column labeled “Cas9 volume”) for wells 1-4 was 50 nM. For wells 5 and 6, 40 μl of cell lysate was prepared from 700 μl E. coli culture of OD₆₀₀ nm=1. Solid rectangles represent the band area that were used to obtain the standard curve and sample immunoblotting intensities. Dashed rectangles represent the band area that was used to correct for background. FIG. 6A. Calculations were performed as in the following examples: In the well with 0.2 μl Cas9 standard, the Cas9 number is: 50 nM×0.2 μl×6.02×10²³=6.02×10⁹). In the well with 3 μl cell lysate added, the cell number is: 8.57×10⁸×0.7×3 μl/40 μl=4.499×10⁷. dCas9s per cell can then be calculated, which is: 2.358×10¹⁰/(4.499×10⁷)=524. The black rectangle marked with a bracket and asterisk is the area presented in FIG. 1G. FIG. 6B. Calculations performed as in FIG. 6A. FIG. 6C. Calculations performed as in FIG. 6A.

FIG. 7. Sensitivity of dCas9*_PhlF to the addition of DAPG. The fold-repression is shown in the absence (black bars) and presence (white bars) of the PhlF inducer DAPG (100 μM). The pSZ_Output and pSZ_PhlF plasmids were used for these experiments (FIGS. 15A-15F). The average of three experiments performed on different days is shown and the error bars indicate the standard deviation.

FIG. 8. Four inducible systems that respond to small molecules. White bars are the output promoter strength without inducers, and black bars are the output promoter strength when each inducer was added (measured with plasmid pSZ_Sensor, FIGS. 16A-16D). The inducer concentrations used to fully induce the promoters (from left to right): 1 mM IPTG, 100 μM vanillic acid, 10 mM Choline, and 10 μM 3OC6-AHL. The average of three experiments performed on different days is shown and the error bars indicate the standard deviation.

FIGS. 9A-9C. Representative histograms corresponding to the cascades. FIG. 9A. sgRNA2. FIG. 9B. sgRNA2 and sgRNA8. FIG. 9C. sgRNA2, sgRNA8, and sgRNA9. FIG. 9D. sgRNA2, sgRNA8, sgRNA9, and sgRNA3. Distributions are shown for the cascades in the presence (+) and absence (−) of inducer (100 μM vanillic acid). These data correspond to FIG. 2D.

FIG. 10. Evaluation of cascades at lower dCas9*_PhlF expression. The same experiments described in FIG. 2D were repeated, but where dCas9*_PhlF was expressed at a lower level. The inducer concentration was 0.5 ng/ml aTc. The average of three experiments performed on different days is shown and the error bars indicate the standard deviation.

FIG. 11. Plasmids with different numbers of sgRNAs. Verification of the sizes of constructs shown in FIG. 3B. M: DNA ladder; N1-N16: Plasmids containing 1 to 16 sgRNAs (FIGS. 16A-16D). These plasmids were all digested with BspHI to linearize the plasmids. Expected sizes of these linearized plasmids after digestion are (from left to right): 2300 bp, 2914 bp, 3351 bp, 3572 bp, 4023 bp, 4467 bp, 4834 bp, 5333 bp, and 5773 bp.

FIG. 12. −/+sgRNA fold-change of the cognate promoter. Each strain was transformed with a plasmid containing the cognate promoter (pSZ_Gate, FIGS. 16A-16D) for each titration sgRNA. −/+sgRNA fold-change of the promoter was then measured by co-transforming each strain with the plasmid (pSZ_Titration, FIGS. 16A-16D) containing all 16 titration sgRNAs. The average of three experiments performed on different days is shown and the error bars indicate the standard deviation.

FIG. 13. Toxicity of expressing multiple sgRNAs. The growth impact of co-expressing multiple sgRNAs was compared and normalized to the strain with no sgRNA expressed, following the same growth assay as in FIG. 1D (Methods). No dCas9 or dCas9*_PhlF was expressed in these experiments. The average of three experiments performed on different days is shown and error bards indicate the standard deviation.

FIG. 14. Sequences of 30 sgRNA and cognate promoters (top to bottom: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 2, 4, 6, 8, 10, 12, 14, 16, 18, 20, 22, 24, 26, 28, 30, 32, 34, 36, 38, 40, 42, 44, 46, 48, 50, 52, 54, 56, 58, and 60). sgRNA is the seed region that targets the cognate promoter (Target sequence) and tracrRNA is the scaffold region of sgRNA. All promoters have the same 30 bp PhlF operator sequence and additional 20 bp random generated spacer sequence (Promoter spacer) (Methods).

FIGS. 15A-15F. Plasmid maps for gate components. FIG. 15A. pSZ_Backgbone: Plasmid used to measure auto-fluorescence. FIG. 15B. pSZ_pTet: Plasmid for measuring pTet promoter strength. FIG. 15C. pSZ_Output: Plasmid for measuring output promoter strength. FIG. 15D. pSZ_Input: Plasmid for measuring input promoter (pTac) strength. FIG. 15E. pSZ_ZFP: Plasmid with fused dCas9*_ZFP complex. FIG. 15F. pSZ_PhlF: Plasmid expressing the fused dCas9*_PhlF.

FIGS. 16A-16E. Plasmid maps for circuit characterization. FIG. 16A. pSZ_Sensor: Four input sensor plasmid used to measure the input gates parameters. FIG. 16B. pSZ_Gate: Plasmid used to measure gate parameters. FIG. 16C. pSZ_NOT1: 1-layer NOT inverter; pSZ_NOT2: 2-layer NOT inverter; pSZ_NOT3: 3-layer NOT inverter; pSZ_NOT4: 4-layer NOT inverter. FIG. 16D. pSZ-RT1 and pSZ-RT2: Plasmids for measuring retroactivity in FIG. 3A. FIG. 16E. pSZ-Titration: Plasmid for expressing sgRNA arrays.

### DETAILED DESCRIPTION

Large synthetic genetic circuits require the simultaneous expression of many regulators. Deactivated Cas9 (dCas9) can serve as a repressor by having a small guide RNA (sgRNA) direct it to bind a promoter. The programmability and specificity of RNA:DNA basepairing simplifies the generation of many orthogonal sgRNAs that, in theory, could serve as a large set of regulators in a circuit. However, dCas9 is toxic in many bacteria, thus limiting how high it can be expressed, and low concentrations are quickly sequestered by multiple sgRNAs. Here, a non-toxic version of dCas9 was constructed by eliminating PAM (protospacer adjacent motif) binding with a R1335K mutation (dCas9*) and recovering DNA binding by fusing it to the PhlF repressor (dCas9*_PhlF). Both the 30 bp PhlF operator and 20 bp sgRNA binding site are required to repress a promoter. The larger region required for recognition mitigates toxicity in Escherichia coli, allowing up to 9600±800 molecules of dCas9*_PhlF per cell before growth or morphology are impacted, as compared to 530±40 molecules of dCas9. Further, PhlF multimerization leads to an increase in average cooperativity from n=0.9 (dCas9) to 1.6 (dCas9*_PhlF). A set of 30 orthogonal sgRNA-promoter pairs were characterized as NOT gates; however, the simultaneous use of multiple sgRNAs leads to a monotonic decline in repression and after 15 are co-expressed the dynamic range is <10-fold. This disclosure introduces a non-toxic variant of dCas9, critical for its use in applications in metabolic engineering and synthetic biology, and exposes a limitation in the number of regulators that can be used in one cell when they rely on a shared resource.

In this study, ZFPs as well as TetR-family homologs were fused to a dCas9 variant that has an impaired ability to recognize PAM sites. The corresponding operators for these DNA binding proteins were placed in proximity to the cognate promoters to increase targeting specificity. Among all the tested DNA binding proteins, fusion with PhlF showed the best repression fold change. Importantly, the fused dCas9-DNA binding protein complex showed significantly reduced toxicity when compare to dCas9, and the resulting gates generated non-linear response curves. These improvements will enable complex genetic circuits to be built when both digital response curves and large amounts of dCas9 protein are needed. Given the large number of orthogonal transcription factors and ever increasing Cas9 complexes being identified, this approach will enable even more complex circuits to be constructed in the future. Moreover, this approach is sufficiently general to apply to many CRISPR/Cas proteins.

Described herein are novel CRISPR/dCas9-based logic gates and methods of regulating expression of an output sequence through the introduction of novel CRISPR/dCas9-based logic gates into a cell.

Compositions of a Synthetic Genetic Circuits and Non-Natural Cells

In one aspect, the components of a synthetic genetic circuit are provided, including a single polynucleotide or a combination of polynucleotides that encode: at least one fusion protein comprising a catalytically-inactive CRISPR/Cas protein fused to a transcription factor, wherein the catalytically-inactive CRISPR/Cas protein comprises a mutated protospacer adjacent motif (PAM) domain (or PAM-interacting domain) and a mutated or absent HNH domain, at least one small guide RNA (i.e., sgRNA), and at least one output sequence whose expression is operably linked to an output promoter, wherein the output promoter comprises a transcription factor operator and a cognate promoter comprising an sgRNA target site and, optionally, a PAM site.

As used herein, the term “genetic circuit” refers to a controllable gene expression system. The term “synthetic genetic circuit” refers to an engineered, non-natural genetic circuit. Genetic circuits function by changing the flow of RNA polymerase on DNA. In some embodiments a synthetic genetic circuits functions by increasing the flow of RNA polymerase at one or more locations. In other embodiments, a synthetic genetic circuit functions by decreasing the flow of RNA polymerase at one or more locations. In still other embodiments, a synthetic genetic circuit functions by increasing the flow of RNA polymerase at one or more locations and decreasing the flow of RNA polymerase at one or more locations.

In some embodiments, the fusion protein(s), sgRNA(s), and output sequence(s) of a synthetic genetic circuit (i.e., “the core elements of the synthetic genetic circuit”) are encoded on a single polynucleotide (e.g., on the same backbone). In some embodiments, the core elements of the synthetic genetic circuit are encoded in any combination on multiple, independent polynucleotides. In some embodiments, the ratios of the core elements of the synthetic genetic circuit are equivalent (e.g., one fusion protein, one sgRNA, and one output sequence). In some embodiments, the ratios of the core elements of the synthetic genetic circuit are not equivalent (e.g., two fusion proteins, eight sgRNAs, and fifteen output sequences). In some embodiments, the core elements of the synthetic genetic circuit include multiple copies of the same fusion protein, sgRNA, or output sequence. In other embodiments, the core elements of the synthetic genetic circuit are each unique (e.g., each fusion protein, sgRNA, and output sequence has a unique composition). In some embodiments, the polynucleotide or combination of polynucleotides of a synthetic genetic circuit are in the form of a circular double stranded DNA (e.g., a viral vector or plasmid). In some embodiments, the components are encoded on plasmid p15A. In other embodiments, the polynucleotides or combination of polynucleotides of a synthetic genetic circuit are in the form of linear double stranded DNA (e.g., genomic DNA). In yet other embodiments, a combination of polynucleotides of a synthetic genetic circuit includes at least one polynucleotide that is in the form of circular double stranded DNA and at least one polynucleotide that is in the form of linear double stranded DNA.

The terms “fusion” or “fusion protein” refer to the combination of two or more polypeptides/peptides in a single polypeptide chain. Fusion proteins typically are produced genetically through the in-frame fusing of the nucleotide sequences encoding for each of the said polypeptides/peptides. Expression of the fused coding sequence results in the generation of a single protein without any translational terminator between each of the fused polypeptides/peptides. Alternatively, fusion proteins also can be produced by chemical synthesis.

In some embodiments of the fusion proteins described herein, the catalytically-inactive CRISPR/Cas protein of the fusion protein is fused to the transcription factor with a C-terminal polypeptide bond. In such embodiments, the C-terminal amino acid of the catalytically-inactive CRISPR/Cas protein is fused to the N-terminal amino acid of the transcription factor. In other embodiments, the catalytically-inactive CRISPR/Cas protein is fused to the transcription factor with an N-terminal polypeptide bond. In such embodiments, the N-terminal amino acid of the catalytically-inactive CRISPR/Cas protein is fused to the C-terminal amino acid of the transcription factor.

In some embodiments, the fusion of the catalytically-inactive CRISPR/Cas protein and the transcription factor is direct (i.e., without any additional amino acids residues between the fused polypeptides/peptides). In other embodiments, the catalytically-inactive CRISPR/Cas protein and the transcription factor of a fusion protein are separated by a linker peptide. As used herein, the term “linker peptide” refers to a polypeptide that serves to connect the CRISPR/Cas protein with the transcription factor of a fusion protein. The length of a linker peptide can vary; for example, the length may be as few as one amino acid or more than one hundred amino acids. Non-limiting examples of linker peptides contemplated herein include flexible linkers, such as Gly-Ser linkers. Such linkers can have the formula Glyₓ-Ser_{y} in which x=1, 2, 3, 4, 5, 6, 7, 8, 9, or 10 and y=1, 2, 3, 4, 5, 6, 7, 8, 9, or 10. In one specific embodiment, x=4 and y=1, such that the linker formula is Gly₄-Ser₁ (SEQ ID NO: 129). The Gly-Ser linker can be replicated n number of times [(Glyₓ-Ser_{y})ₙ], for example, wherein n=1, 2, 3, 4, 5, 6, 7, 8, 9, 10, 15, 20, 25, or 30. Additional flexible linkers include, e.g., (Gly)₆ (SEQ ID NO: 130), (Gly)₈ (SEQ ID NO: 131), etc. Additional linkers include rigid linkers (e.g., (EAAAK)₃ (SEQ ID NO: 132), A(EAAAK)₄ALEA(EAAAK)₄A (SEQ ID NO: 133), PAPAP (SEQ ID NO: 134), etc.) and cleavable linkers (e.g., disulfide, VSQTSKLTR↓AETVFPDV (SEQ ID NO: 135), RVL↓AEA (SEQ ID NO: 136); EDVVCC↓SMSY (SEQ ID NO: 137); GGIEGR↓GS (SEQ ID NO: 138); GFLG↓ (SEQ ID NO: 139), etc. (cleavage site marked by “↓”)). Any of the linkers can be naturally-occurring or synthetic.

As used herein, the term “CRISPR/Cas protein” refers to an RNA-guided DNA endonuclease, including, but not limited to, Cas9, Cpf1, C2c1, and C2c3 and each of their orthologs and functional variants. The amino acid sequence of exemplary Streptococcus pyogenes serotype M1 Cas9 is provided below, which serves as a reference for the Cas9 mutation numbering described herein:

Cas 9
(SEQ ID NO: 128)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

As used herein, the term “functional variants” includes polypeptides which are at least about 70% identical, at least about 80% identical, at least about 90% identical, at least about 95% identical, at least about 98% identical, at least about 99% identical, at least about 99.5% identical, or at least about 99.9% identical to a protein's native amino acid sequence (i.e., wild-type amino acid sequence) and which retain functionality. The term “functional variants” also includes polypeptides which are shorter or longer than a protein's native amino acid sequence by 1, 2, 3, 4, 5, 6, 7, 8, 9, 10, 11, 12, 13, 14, 15, 16, 17, 18, 19, 20, 21, 22, 23, 24, 25, 30, 40, 50, 75, 100 amino acids or more and which retain functionality. In the context of a CRISPR/Cas protein variant, the term “retain functionality” refers to a variant's ability to bind RNA at least about 5%, 10%, 20%, 25%, 30%, 40%, 50%, 60%, 70%, 75%, 80%, 90%, 100%, or more than 100% as efficiently as the respective non-variant (i.e., wild-type) CRISPR/Cas protein. Methods of measuring and comparing the efficiency of RNA binding are known to those skilled in the art.

The term “catalytically-inactive CRISPR/Cas protein” as used herein refers to a CRISPR/Cas protein variant or mutant that lacks endonuclease activity (i.e., the ability to cleave double stranded DNA). For example, catalytically-inactive Cas9 mutants have been generated through incorporation of various mutations (e.g., D10 mutants) mutations (Jinek et al., Science 337, 816-21 (2012)).

The terms “PAM domain” or “PAM-interacting domain” are used interchangeably herein to refer to a domain of a CRISPR/Cas protein that is responsible for recognition of protospacer adjacent motifs (PAMs or PAM sites). The term “mutated PAM domain” refers to any point mutation, insertion, deletion, frameshift, or mis sense mutation or any combination of these mutations that decreases a CRISPR/Cas protein's ability to recognize a PAM site by at least 20%, 25%, 30%, 40%, 50%, 60%, 70%, 75%, 80%, 90% or up to 100% relative to the respective non-variant (i.e., wild-type) CRISPR/Cas protein. For example, Cas9 R1335 point mutations (e.g., R1335K) decrease Cas9's ability to recognize PAM sites. Methods of measuring and comparing PAM recognition are known to those skilled in the art.

The term “HNH domain” refers to a protein endonuclease domain. The term “mutated HNH domain” refers to any point mutation, insertion, deletion, frameshift, or missense mutation or any combination of these mutations to a CRISPR/Cas protein's HNH domain. For example, in some embodiments, the mutation of the CRISPR/Cas HNH domain consists of the deletion of the entire domain and its replacement by an amino acid linker sequence (e.g., GGSGGS, SEQ ID NO: 127). As used herein, the term “amino acid linker sequence” refers to a polypeptide that serves to replace the HNH domain of a CRISPR/Cas protein. The length of an amino acid linker can vary; for example, the length of an amino acid linker may as few as one amino acid or more than one hundred amino acids. The term “absent,” in the context of an HNH domain, refers to and encompasses CRISPR/Cas proteins that inherently lack an HNH domain (e.g., Cpf1, C2c1, and C2c3).

In some embodiments, the catalytically-inactive CRISPR/Cas protein of a fusion protein possesses a functional RuvC domain. The term “RuvC domain” refers to a protein endonuclease domain. “Possesses a functional RuvC domain” refers to a native or wild-type RuvC domain, or any mutation thereof, that retains the catalytically-inactive CRISPR/Cas protein's ability to regulate the expression of an output promoter. In some embodiments, the catalytically-inactive CRISPR/Cas protein of a fusion protein possess a native or wild-type RuvC domain. The terms “native RuvC domain” or “wild-type RuvC domain” refer to an RuvC domain composed entirely of an amino acid sequence that is found in nature.

In some embodiments, the catalytically-inactive CRISPR/Cas protein of a fusion protein consists of amino acids 1 to 1368 of Cas9, wherein the Cas9 amino acid sequence contains D10A and R1335K mutations and the Cas9 amino acids 768 to 919 are replaced by a GGSGGS (SEQ ID NO: 127) amino acid linker sequence.

Cas9 orthologs have been described in various species, including, but not limited to Bacteroides coprophilus (e.g., NCBI Reference Sequence: WP_008144470.1), Campylobacter jejuni susp. jejuni (e.g., GeneBank: AJP35933.1), Campylobacter lari (e.g., GeneBank: AJD02827.1), Fancisella novicida (e.g., UniProtKB/Swiss-Prot: A0Q5Y3.1), Filifactor alocis (e.g., NCBI Reference Sequence: WP_083799662.1), Flavobacterium columnare (e.g., GeneBank: AMA50561.1), Fluviicola taffensis (e.g., NCBI Reference Sequence: WP_013687888.1), Gluconacetobacter diazotrophicus (e.g., NCBI Reference Sequence: WP_041249387.1), Lactobacillus farciminis (e.g., NCBI Reference Sequence: WP_010018949.1), Lactobacillus johnsonii (e.g., GeneBank: KXN76786.1), Legionella pneumophila (e.g., NCBI Reference Sequence: WP_062726656.1), Mycoplasma gallisepticum (e.g., NCBI Reference Sequence: WP_011883478.1), Mycoplasma mobile (e.g., NCBI Reference Sequence: WP_041362727.1), Neisseria cinerea (e.g., NCBI Reference Sequence: WP_003676410.1), Neisseria meningitidis (e.g., GeneBank: ODP42304.1), Nitratifractor salsuginis (e.g., NCBI Reference Sequence: WP_083799866.1), Parvibaculum lavamentivorans (e.g., NCBI Reference Sequence: WP_011995013.1), Pasteurella multocida (e.g., GeneBank: KUM14477.1), Sphaerochaeta globusa (e.g., NCBI Reference Sequence: WP_013607849.1), Streptococcus pasteurianus (e.g., NCBI Reference Sequence: WP_061100419.1), Streptococcus thermophilus (e.g., GeneBank: ANJ62426.1), Sutterella wadsworthensis (e.g., NCBI Reference Sequence: WP_005430658.1), and Treponema denticola (e.g., NCBI Reference Sequence: WP_002684945.1).

In some embodiments, “Cas9” refers to any one of the Cas9 orthologs described herein, including functional variants thereof or suitable Cas9 endonucleases and sequences that are apparent to those of ordinary skill in the art.

The term “transcription factor” refers to any polypeptide that is capable of binding DNA and that, when bound, regulates output gene expression. “Regulates output gene expression” refers to a change (increase or decrease) of at least 5%, 10%, 20%, 25%, 30%, 40%, 50%, 60%, 70%, 75%, 80%, 90%, 100%, 500%, 1000%, 10,000% or more than 10,000% in the level of output gene expression relative to the level of expression in the absence of the transcription factor. Methods of measuring and comparing gene expression are known to those skilled in the art. In some embodiments, the transcription factor activates or increases a genetic circuit's output gene expression. In other embodiments, the transcription factor represses or decreases a genetic circuit's output gene expression.

In some embodiments of the fusion proteins described herein, the transcription factor of a fusion protein is PhlF or an ortholog or functional variant thereof. In other embodiments, the transcription factor of a fusion protein is BM3RI or an ortholog or functional variant, thereof. In other embodiments, the transcription factor of a fusion protein is a ZFP protein or an ortholog or functional variant, thereof. In the context of a PhlF, BM3RI, or ZFP protein variant, the term “retain functionality” refers to a variant's ability to repress (or decrease) gene expression at least about 5%, 10%, 20%, 25%, 30%, 40%, 50%, 60%, 70%, 75%, 80%, 90%, 100%, or more than 100% as efficiently as the respective wild-type protein. Methods of measuring and comparing gene expression are known to those skilled in the art.

As used herein, the terms “small guide RNA” or “sgRNA” refer to a nucleic acid molecule that has a sequence that complements an sgRNA target site, which mediates binding of the CRISPR/Cas-RNA complex to the sgRNA target site, providing the specificity of the CRISPR/Cas-RNA complex. Typically, guide RNAs that exist as single RNA species comprise two domains: (1) a “guide” domain that shares homology to a target nucleic acid (e.g., directs binding of a CRISPR/Cas complex to a target site); and (2) a “direct repeat” domain that binds a CRISPR/Cas protein. In this way, the sequence and length of a small guide RNA may vary depending on the specific sgRNA target site and/or the specific CRISPR/Cas protein (Zetsche et al. Cell 163, 759-71 (2015)).

In some embodiments, a genetic circuit comprises a single sgRNA. In other embodiments, a genetic circuit comprises two unique sgRNAs, wherein both sgRNAs can be fully expressed and independently repress two promoters without incurring significant negative effects on repression due to resource sharing (e.g., insufficient dCas9-fusion protein). In some embodiments, a genetic circuit comprises more than two unique sgRNAs (e.g., 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 unique sgRNAs).

The term “output sequence” as used herein refers to an expressible nucleotide sequence that is operably linked to an output promoter of a synthetic genetic circuit. In some embodiments, the expressible nucleotide sequence of an output sequence comprises the nucleotide sequence of a non-coding RNA (e.g., a tRNA, rRNA, miRNA, siRNA, shRNA, sgRNA, piRNA, snoRNA, snRNA, exRNA, scaRNA, tracrRNA, lncRNA, riboswitch, or ribozyme). In some embodiments, the expressible nucleotide sequence of an output sequence comprises the nucleotide sequence of an RNA that encodes for a protein product (i.e., a mRNA). In some embodiments, the protein product is a therapeutic protein. In some embodiments, the protein product is a detectable protein, such as a fluorescent protein. In some embodiments, a genetic circuit comprises more than two unique output sequences (e.g., 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 unique output sequences).

The term “operably linked” as used herein refers to a relationship between an output promoter and an output sequence wherein the position of the output promoter relative to the output sequence is such that the output promoter is able to influence the expression of the output sequence. The term “influence the expression” refers to output sequence expression level changes (increases or decreases) of at least 5%, 10%, 20%, 25%, 30%, 40%, 50%, 60%, 70%, 75%, 80%, 90%, 100%, 500%, 1000%, 10,000% or more than 10,000% relative to output sequence expression levels in the absence of the output promoter. Methods of measuring and comparing promoter functionality are known to those skilled in the art.

As used herein, the term “transcription factor operator” refers to the DNA sequence that a transcription factor binds to; for example, the PhlF operator is the DNA sequence that PhlF binds to. In some embodiments, the transcription factor operator is positioned 3′ to the cognate promoter. In other embodiments, the transcription factor operator is positioned 5′ to the cognate promoter. In some embodiments, the transcription factor operator and the cognate promoter are oriented on the same DNA strand. In other embodiments, the transcription factor operator and the cognate promoter are oriented on complementary DNA strands. In some embodiments, the transcription factor operator and the cognate promoter sequence are separated by 0 to 20 base pairs.

As used herein, the term “cognate promoter” refers to a DNA sequence that interacts with a CRISPR/Cas complex. In some embodiments, the cognate promoter consists of an sgRNA target site. The term “sgRNA target” refers to a sequence that is complementary to a CRISPR/Cas protein's complexed sgRNA. In some embodiments, the sgRNA target site of at least one of the output promoters comprises the sgRNA target site of at least one sgRNA whose expression is under the control of an inducible promoter. Examples of inducible promoters are known to those having skill in the art. In some embodiments, an inducible promoter is a chemically inducible promoter (e.g., pTet, pTac, or pVan), a temperature inducible promoter, or a light inducible promoter. In some embodiments, the inducer of an inducible promoter is a small molecule (e.g., aTc, IPTG, or vanillic acid). In other embodiments, the inducer is a large molecule (e.g., a protein or non-coding RNA).

In some embodiments, the cognate promoter comprises an sgRNA target site and a PAM site. The term “PAM” or “PAM site” are used interchangeably herein to refer to a short nucleotide sequence, generally 2-6 base pairs in length, that is recognized by a CRISPR/Cas protein; for example, Cas9 primarily recognizes NGG elements as PAM sites, though it has been shown that it can also inefficiently recognize other PAM sites (e.g., NAG or NGA) (Zhang et al., Sci. Rep. 4, 1-5 (2014); Hsu et al., Nat. Biotechnol. 31, 827-32 (2013)). PAM sites can vary between CRISPR/Cas proteins and each protein's species of origin. In some embodiments, the cognate promoter lacks a PAM site.

In some embodiments, the transcription factor operator and the cognate promoter of the output promoter are on the same DNA strand. In other embodiments, the transcription factor operator and the cognate promoter of the output promoter are on complementary DNA strands. In some embodiments, the transcription factor operator and the cognate promoter of the output promoter are separated by 0 to 20 base pairs.

In some embodiments, the output promoter also comprises minimal gene promoter elements. In some embodiments, these minimal gene promoter elements provide for basal or constitutive expression of an output sequence which can be activated or repressed by the binding of a fusion protein to the output promoter.

In some embodiments, the catalytically-inactive CRISPR/Cas protein consists of amino acids 1 to 1368 of Cas9, wherein the Cas9 amino acid sequence contains D10A and R1335K mutations and the Cas9 amino acids 768 to 919 are replaced by a GGSGGS (SEQ ID NO: 127) amino acid linker sequence, the transcription factor is PhlF, the catalytically-inactive CRISPR/Cas protein is fused to PhlF with a C-terminal polypeptide bond, the transcription factor operator of the output promoter is a PhlF operator, and the PhlF operator and the cognate promoter sequence of the output promoter are separated by 0 to 20 base pairs.

In some embodiments, the single polynucleotide or the combination of polynucleotides of a genetic circuit encode: (a) at least one fusion protein comprising a catalytically-inactive CRISPR/Cas protein fused to a transcription factor, wherein the catalytically-inactive CRISPR/Cas protein comprises a mutated PAM domain and a mutated or absent HNH domain; (b) between two and thirty unique sgRNAs, wherein the expression of at least one of the unique sgRNAs is under the control of an inducible promoter; and (c) between one and twenty-nine output sequences, each of whose expression is operably linked to an independent output promoter, wherein at least two of the output promoters comprise a transcription factor operator and a cognate promoter comprising a unique sgRNA target site and, optionally, a PAM site, and wherein: (i) the unique sgRNA target site of each output promoter comprising an sgRNA target site comprises an sgRNA target site of one of the sgRNAs in (b); and (ii) the unique sgRNA target site of at least one of the output promoters comprises the sgRNA target site of the at least one sgRNA under the control of an inducible promoter in (b).

In some embodiments, the genetic circuit is encoded on a single polynucleotide. In some embodiments, the single polynucleotide is a plasmid. In some embodiments, the genetic circuit is encoded on more than one polynucleotides. In some embodiments, at least one of the more than one polynucleotides is a plasmid.

In another aspect, a polynucleotide or combination of polynucleotides are provided. In some embodiments, the polynucleotide or combination of polynucleotides comprise(s) the nucleotide sequence of a genetic circuit described above. Also disclosed herein are compositions comprising the polynucleotide or combination of polynucleotides.

In another aspect, the disclosure relates to non-natural cells comprising a genetic circuit as described above or a polynucleotide or combination of polynucleotides as described above. The term “non-natural cells,” as used herein, relates to a cell that has been engineered to be different from its natural counterpart or the cell from which it is derived.

In some embodiments, a non-natural cell comprises a genetic circuit that comprises at least one output promoter comprising an sgRNA target site of at least one sgRNA whose expression is under the control of an inducible promoter. In some embodiments, the source of the inducer of the inducible promoter is outside of the cell (e.g., a small molecule inducer, such as aTc, IPTG, or Vanillic acid). In other embodiments, the source of the inducer of the inducible promoter is within the cell. For example, the non-natural cell may respond to an external or internal stimulus via the production of a molecule (e.g., a protein, non-coding RNA, etc.) that is the inducer of the inducible promoter.

Compositions of Fusion Proteins

In another aspect, compositions of fusion proteins are provided, including a catalytically-inactive Cas9 protein linked by a C-terminal polypeptide bond to PhlF, wherein the catalytically-inactive Cas9 protein comprises a mutated PAM domain, a mutated HNH domain, and a functional RuvCI domain, and optionally, the catalytically-inactive Cas9 protein and the PhlF protein are separated by a linker peptide. Relevant definitions and term usages described in “Components of a Synthetic Circuit” above apply to this section, as well.

In some embodiments, the mutation of the Cas9 HNH domain consists of the deletion of the entire domain and its replacement by an amino acid linker sequence. In some embodiments, the catalytically-inactive Cas9 protein amino acid sequence contains D10A and R1335K mutations and the Cas9 amino acids 768 to 919 are replaced by a GGSGGS (SEQ ID NO: 127) amino acid linker sequence.

The compositions of fusion proteins have, in some embodiments, a single type of fusion protein (i.e., all the fusion proteins in the composition have the same amino acid sequence). In other embodiments, however, the fusion protein compositions include two or more types of fusion proteins (i.e., a “cocktail” of fusion proteins). For example, fusion proteins of a composition may include fusion proteins that have: (1) catalytically-inactive Cas9 proteins and/or PhlF transcription factors from different species; (2) catalytically-inactive Cas9 proteins of the same species that have different mutations and/or amino acid linker sequences; (3) PhlF transcription factors of the same species that have different mutations; and/or (4) different linker peptide sequences.

In some embodiments, the fusion proteins in a fusion protein composition may include non-canonical amino acids (e.g., amino acid phosphorylation, methylation, acetylation, amidation, isomerization, hydroxylation, sulfonation, and cysteine oxidation and nitrosylation).

In some embodiments, the compositions also comprise an sgRNA or a combination of sgRNAs that can be bound by the fusion proteins of the composition. In some embodiments, the compositions include diluents of various: buffer content (e.g., Tris-HCl, Tris Base, acetate, phosphate), pH and ionic strength; additives such as detergents and solubilizing agents (e.g., Triton X-100, Tween 80, Polysorbate 80), anti-oxidants (e.g., DTT, ascorbic acid, sodium metabisulfite), preservatives (e.g., Thimersol, benzyl alcohol, sodium azide), and stabilizers (e.g., glycerol, mannitol, trehalose). In some embodiments, the protein compositions are incorporated into particulate preparations of polymeric compounds (e.g., polylactic acid, polyglycolic acid, etc.) or into liposomes.

In some embodiments, the compositions are provided in a dry, solid form (e.g., lyophilized compositions). In other embodiments, the compositions are provided in a liquid form. In some embodiments, the compositions are frozen. In some embodiments, the fusion compositions include packaging material and a container, wherein the packaging material comprises a label that indicates how the composition can be stored over various periods of time and the conditions under which the composition may be used.

Composition of Polynucleotides

In another aspect, compositions of polynucleotides encoding for fusion proteins are provided, including compositions of a polynucleotide encoding for any fusion protein encompassed above in “Compositions of Fusion Proteins.” For example, in some embodiments, a polynucleotide encodes for a catalytically-inactive Cas9 protein linked by a C-terminal polypeptide bond to PhlF, wherein the catalytically-inactive Cas9 protein comprises a mutated PAM domain, a mutated HNH domain, and a functional RuvCI domain, and optionally, the catalytically-inactive Cas9 protein and the PhlF protein are separated by a linker peptide.

The polynucleotide compositions have, in some embodiments, a single type of polynucleotide (i.e., each polynucleotide in the composition consists of the same nucleic acid sequence). In other embodiments, however, the polynucleotide compositions include two or more types of polynucleotides (i.e., a “cocktail” of polynucleotides). For example, polynucleotides of a composition may include polynucleotides that encode for: (1) catalytically-inactive Cas9 proteins and/or PhlF transcription factors from different species; (2) catalytically-inactive Cas9 proteins of the same species that have different mutations and/or amino acid linker sequences; (3) PhlF transcription factors of the same species that have different mutations; and/or (4) different linker peptide sequences. In some embodiments, the polynucleotides that encode for the fusion proteins also encode for one or more sgRNAs and/or one or more output sequences whose expression is operably linked to an output promoter, wherein the output promoter comprises a transcription factor operator and a cognate promoter comprising an sgRNA target site and, optionally, a PAM site. In some embodiments, the composition of polynucleotides includes additional, independent polynucleotides that encode for one or more sgRNAs and/or one or more output sequences whose expression is operably linked to an output promoter, wherein the output promoter comprises a transcription factor operator and a cognate promoter comprising an sgRNA target site and, optionally, a PAM site.

In some embodiments, the polynucleotide composition may include non-canonical nucleotides such as inosine, thiouridine, or pseudouridine. In some embodiments, the polynucleotide composition may include chemically modified nucleotides. Examples of chemically modified oligonucleotides or polynucleotides are well known in the art. For example, the naturally occurring phosphodiester backbone of an oligonucleotide or polynucleotide can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labelled with a fluorescent moiety (e.g., fluorescein or rhodamine) or other label (e.g., biotin).

In some embodiments, the compositions also comprise an sgRNA or a combination of sgRNAs. In some embodiments, the compositions include diluents of various buffer content (e.g., Tris-HCl, Tris Base, acetate, phosphate), pH and ionic strength. In some embodiments, the polynucleotide compositions are incorporated into particulate preparations of polymeric compounds (e.g., polylactic acid, polyglycolic acid, etc.) or into liposomes.

In some embodiments, the compositions of polynucleotides are in a dry, solid form (e.g., lyophilized compositions). In other embodiments, the compositions of polynucleotides are in liquid form. In some embodiments, the compositions of polynucleotides are frozen. In some embodiments, the compositions of polynucleotides include packaging material and a container, wherein the packaging material comprises a label that indicates how the composition can be stored over various periods of time and the conditions under which the composition may be used.

Methods of Regulating Expression of a Genetic Circuit's Output Sequence

In another aspect, methods of regulating expression of a genetic circuit's output sequence are described, including the introduction of a synthetic genetic circuit into a cell. This aspect embodies the cellular introduction of the synthetic genetic circuit compositions encompassed above in “Components of a Synthetic Circuit.”

As used herein, the term “introducing the genetic circuit” refers to any mechanism whereby a polynucleotide or combination of polynucleotides can be transferred from a cell's exterior to that cell's interior, in which the cell remains viable. Methods of introducing polynucleotides into a cell are known to those of ordinary skill in the art and include, but are not limited to, electroporation, transfection (e.g., heat-shock-mediated transfection, laser transfection, lipofectamine-mediated transfection, liposomal transfection), transformation, microinjection, nuclear injection, biolistics, gene guns, gene therapy, and gene transfer.

“Cell” as used herein may refer to a prokaryotic cell, a eukaryotic cell, or a synthetic cell (i.e., a minimal cell or an artificial cell). “Prokaryotic cells” include bacteria and archaea. In some embodiments the prokaryotic cell is a bacteria of a phyla selected from Actinobacteria, Aquificae, Armatimonadetes, Bacteroidetes, Caldiserica, Chlamydiae, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Nitrospirae, Planctomycetes, Proteobacteria, Spirochaetes, Synergistets, Tenericutes, Thermodesulfobacteria, and Thermotogae. In some embodiments the prokaryotic cell is an archaea of a phyla selected from Euryarcheota, Crenarcheota, Nanoarchaeota, Thaumarchaeota, Aigarchaeota, Lokiarchaeota, Thermotogae, and Tenericutes. In some embodiments the eukaryotic cell is a member of a kingdom selected from Protista, Fungi, Plantae, or Animalia. In some embodiments the cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus. In other embodiments the cell is an archaeal cell, such as Methanosphaera spp., Methanothermus spp., Methanomicrobium spp., Methanohalobium spp., Methanimicrococcus spp., Methanocalculus spp., Haloferax spp., Halobacterium spp., Halococcus spp., Halorubrum spp., Haloterrigena spp., Thermoplasma spp., Thermoproteus spp., Chaetomium spp., Thermomyces spp., Brevibacillus spp., and Sulfolobus spp. In other embodiments, the cell is a fungal cell such as a yeast cell, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp., and industrial polyploid yeast strains. Preferably the yeast strain is a S. cerevisiae strain or a Yarrowia spp. strain. Other examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In other embodiments, the cell is a mammalian cell, an algal cell, or a plant cell. As used herein, “synthetic cell” refers to an engineered cell that mimics one or more functions or structure of a biological cell. In some embodiments, the cell exists independent of other cells (i.e., is single cellular). In other embodiments the cell exists as part of a multicellular organism (e.g., part of a tissue or organ). For example, a cell may be located in a transgenic animal or transgenic plant.

A relevant synthetic genetic circuit that can be introduced into a cell may comprise a single layer input gate. For example, in some embodiments, a genetic circuit may comprise a fusion protein (e.g., a catalytically-inactive Cas9 protein linked by a C-terminal polypeptide bond to PhlF) whose expression is controlled by an inducible promoter (e.g., aTc inducible pTet promoter), an sgRNA whose expression is controlled by a different inducible input promoter (e.g., IPTG inducible pTac promoter), an output promoter that is targeted by fusion protein-sgRNA complexes, and a gene controlled by the output promoter. In some embodiments, these parts are integrated on the same backbone (e.g., p15A) to avoid plasmid variation. Expression and production of the fusion protein and the sgRNA can be stimulated via cellular administration of the appropriate inducers. The fusion proteins and sgRNAs that are produced then form complexes that target the output promoter. The interaction between a fusion protein-sgRNA complex and an output promoter (i.e., the interaction between the transcription factor of the fusion protein with its operator and the interaction between the catalytically-inactive CRISPR/Cas protein of the fusion protein with the sgRNA and the cognate promoter) results in the regulation (i.e., an increase or decrease) of the output gene's expression levels.

A synthetic genetic circuit may also comprise multiple layers. For example, in some embodiments, a genetic circuit with two layers may comprise a fusion protein (e.g., a catalytically-inactive Cas9 protein linked by a C-terminal polypeptide bond to PhlF) whose expression is controlled by an inducible promoter (e.g., aTc inducible pTet promoter), an sgRNA(a) whose expression is controlled by a different inducible input promoter (e.g., vanillic acid inducible pVanR promoter), an output promoter(a) that is targeted and repressed by fusion protein-sgRNA(a) complexes, an sgRNA(b) whose expression is controlled by the output promoter(a), an output promoter(b) that is targeted and repressed by fusion protein-sgRNA(b) complexes, and an output gene whose expression is controlled by the output promoter(b). In some embodiments, these parts may be integrated on the same backbone to avoid plasmid variation. Expression and production of the fusion protein and the sgRNA(a) can be stimulated via cellular administration of the appropriate inducer. The fusion proteins and sgRNA(a)s that are produced then form complexes that target and repress the output promoter(a). The interaction between a fusion protein-sgRNA(a) complex and an output promoter(a) results in repression of sgRNA(b) expression levels. Because sgRNA(b) expression is repressed, fewer fusion protein-sgRNA(b) complexes interact with and repress output promoter(b). Thus, the output gene's expression levels increase.

In another example, a synthetic genetic circuit with three layers may comprise, in some embodiments, a fusion protein whose expression is controlled by an inducible promoter, an sgRNA(a) whose expression is controlled by a different inducible input promoter, an output promoter(a) that is targeted and repressed by fusion protein-sgRNA(a) complexes, an sgRNA(b) whose expression is controlled by the output promoter(a), an output promoter(b) that is targeted and repressed by fusion protein-sgRNA(b) complexes, an sgRNA(c) whose expression is controlled by the output promoter(b), an output promoter(c) that is targeted and repressed fusion protein-sgRNA(c) complexes, and an output gene whose expression is controlled by the output promoter(c). In some embodiments, these parts are integrated on the same backbone to avoid plasmid variation. Expression and production of the fusion protein and the sgRNA(a) can be stimulated via cellular administration of the appropriate inducer. The fusion proteins and the sgRNA(a)s that are produced then form complexes that target and repress the output promoter(a). The interaction between a fusion protein-sgRNA(a) complex and an output promoter(a) results in repression of sgRNA(b) expression levels. Because sgRNA(b) expression is repressed, fewer fusion protein-sgRNA(b) complexes interact with and repress output promoter(b). Thus expression of sgRNA(c) increases. The interaction between a fusion protein-sgRNA(c) complex and an output promoter(c) results in repression of the output gene's expression levels.

In other embodiments, a synthetic genetic circuit comprises four or more layers. The complexity and diversity of the synthetic genetic circuits embodied herein can be selected as needed for particular tasks and outcomes. For example, in some embodiments, a multilayer synthetic genetic circuit comprises multiple input gates.

While the cellular concentrations of the components utilized in this method (e.g., the polynucleotides, the fusion proteins generated via translation of RNAs produced from the polynucleotides, and sgRNAs generated via transcription of the polynucleotides) may vary, the methods can utilize any effective amount of the components. “Any effective amount of the components” refers to any amount that, when combined, results in the regulation of output gene expression or the change (increase or decrease) of at least 5%, 10%, 20%, 25%, 30%, 40%, 50%, 60%, 70%, 75%, 80%, 90%, 100%, 500%, 1000%, 10,000% or more than 10,000% in the level of output gene expression relative to the level of expression in the absence of the combination of components. For example, in some embodiments, the cellular concentration of fused dCas9*-PhlF complex is about 5000 molecules per cell.

### EXAMPLES

Methods and Materials

Strains and media.

All cloning was performed in Escherichia coli NEB 10-beta (New England Biolabs, # C3019) and cells were grown in LB Miller broth (Difco, MI, #90003-350). The measurements experiments were done in E. coli K-12 MG1655* [F-λ-ilvG-rfb-50 rph-1 Δ(araCBAD) Δ(LacI)] (Nielsen A. A. and Voigt C. A., Mol. Syst. Biol., 2014. 10(763): 1-11; Blattner F. R., et al., Science, 1997 Sep. 5; 277(5331): 1453-62), and MOPS EZ Rich Defined Medium was used (Teknova, # M2105) with 0.2% glucose (Thermo Fisher Scientific, #156129) as carbon source for cell growth. Ampicillin (100 μg/ml, GoldBio, # A-301-5), kanamycin (50 μg/ml, GoldBio, # K-120-5), and spectinomycin sulfate (50 μg/ml, GoldBio, # S-140-5) were used to maintain plasmids when appropriate.

Induction assays.

Individual colonies were inoculated into 150 μl MOPS EZ Rich Defined Medium with appropriate antibiotics and then grown overnight (˜16 hours) in 96-well plates (Nunc, Roskilde, Denmark, #249952) at 1,000 rpm and 37° C. on a plate shaker (ELMI, # DTS-4). Cultures were diluted 1000-fold by adding 2 μl of culture to 198 μl media, and then 15 μl of that dilution to 135 μl media, and grown with the same shaking condition for 3 hours. At this point, cells were diluted 3000-fold by adding 2 μl of culture to 198 μl media, and then 5 μl of that dilution to 145 μl media with inducers and antibiotics as needed, and then were grown under the same conditions for 6 hours.

Flow cytometry analyses.

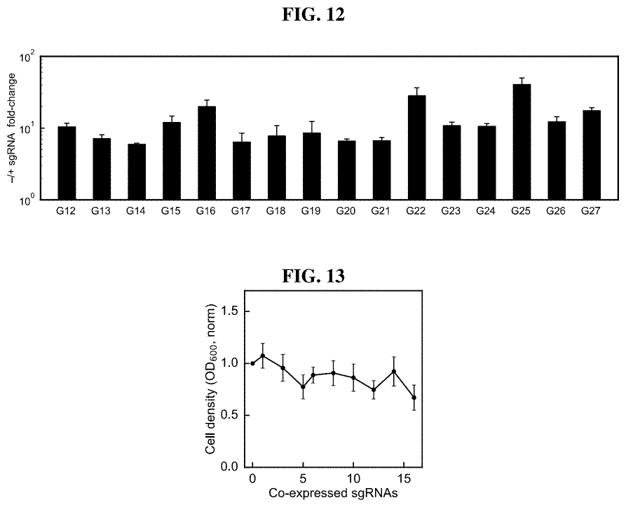

Aliquots of 40 μl of media containing cells were collected and added to 160 μl phosphate-buffered saline with 1 mg/ml kanamycin to stop translation and arrest cell growth. The LSRII Fortessa flow cytometer (BD Biosciences, San Jose, Calif.) was used to quantify the fluorescent protein production. The software FlowJo v10 (TreeStar, Inc., Ashland, Oreg.) was used to gate the events by forward and side scatter, and at least 10,000 events were collected for each sample. The geometric mean of each sample was calculated. The autofluorescence of white cells was subtracted, defined as the geometric mean of a strain harboring an empty backbone (pSZ_Backbone, FIGS. 13A-13F) grown under identical conditions. The fold-repression is measured as the uninduced fluorescence values divided by the induced fluorescence values. In FIG. 12, when sgRNA was expressed on a separate plasmid to repress the cognate promoter, the repression is defined as −/+sgRNA fold-change. And it is measured as without the sgRNA array plasmid divided by with the sgRNA array plasmid fluorescence values.

Growth Assay.

Individual colonies were inoculated into 150 μl MOPS EZ Rich Defined Medium with appropriate antibiotics and then grown overnight (˜16 hours) in 96-well plates (Nunc, Roskilde, Denmark, #249952) at 1,000 rpm and 37° C. on a plate shaker (ELMI, # DTS-4). Cultures were diluted 1000-fold by adding 2 μl of culture to 198 μl media, and then 15 μl of that dilution to 135 μl media, and grown with the same shaking condition for 3 hours. After the 3 hours step, the cultures were diluted 3000-fold by adding 2 μl of culture to 198 μl media, and then 5 μl of that dilution to 145 μl media with appropriate antibiotics and different inducers concentrations. The dilutions were made in 96-well plates (Nunc, Roskilde, Denmark, #165305) and grown at 1,000 rpm and 37° C. for 6 hours. The optical density at 600 nm was measured on a Synergy H1 plate reader (BioTek, Winooski, Vt.) and the background of MOPS EZ Rich Defined Medium was subtracted. The measured values were then normalized to the un-induced samples (0 ng/ml aTc).

Microscopy.

After 6 hours growth in the growth assay experiments, aliquots (2 μl) of cultures were collected. Microscopic images of these cultures were then taken on the Axiovert 200m microscope (Carl Zeiss, Oberkochen, Germany).

Numbers of cells per ml.

Colonies were inoculated into 150 μl MOPS EZ Rich Defined Medium with appropriate antibiotics and then grown overnight (˜16 hours). The next day, these cultures were diluted by adding 1 μl culture into 1 ml fresh media. After 5 hours of growth (1,000 rpm and 37° C.), the culture density was measured and diluted to different OD₆₀₀ nm. The cultures at different OD₆₀₀ nm were then diluted 2×10⁷-fold and plated on LB agar. Colony numbers were then counted after overnight growth at 37° C.

Quantification of dCas9.

Colonies were inoculated into 150 μl MOPS EZ Rich Defined Medium with appropriate antibiotics and then grown overnight (˜16 hours). The next day, these cultures were diluted by adding 1 μl culture into 1 ml fresh media containing inducer (2.5 ng/ml or 0.7 ng/ml aTc). After 5 hours of growth (1,000 rpm and 37° C.), the culture density was measured and adjusted to OD_{600 nm}=1 with MOPS EZ Rich Defined Medium. 700 μl of the adjusted culture for each strain was centrifuged at 12,000 rpm for 1 min. The supernatant was discarded and cell pellet was re-suspended in 40 μl lysis buffer (100 mM NaCl, 25 mM TrisHCl, pH 8.0) containing 0.2% β-mercaptoethanol (Sigma-Aldrich, # M6250). The samples were boiled at 100° C. for 5 min, after which 3 μl of the dCas9 sample and 0.75 μl of the dCas9*_PhlF sample were added to lysis buffer to a final volume of 20 μl.

To prepare the standard curve, 2 μl of purchased Cas9 complex (New England Biolabs, # M0386S) was added to 38 μl lysis buffer. Then, different amounts (0.2 μl, 1 μl, 3 μl, 5 μl) of the diluted Cas9 standard, 3 μl WT lysate, and lysis buffer were added to each sample to a total volume of 20 μl.

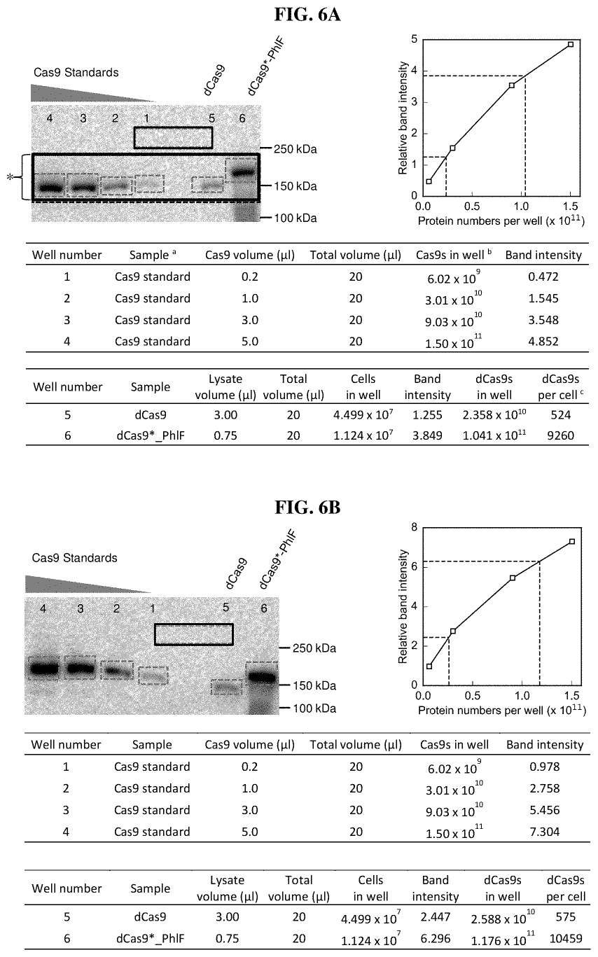

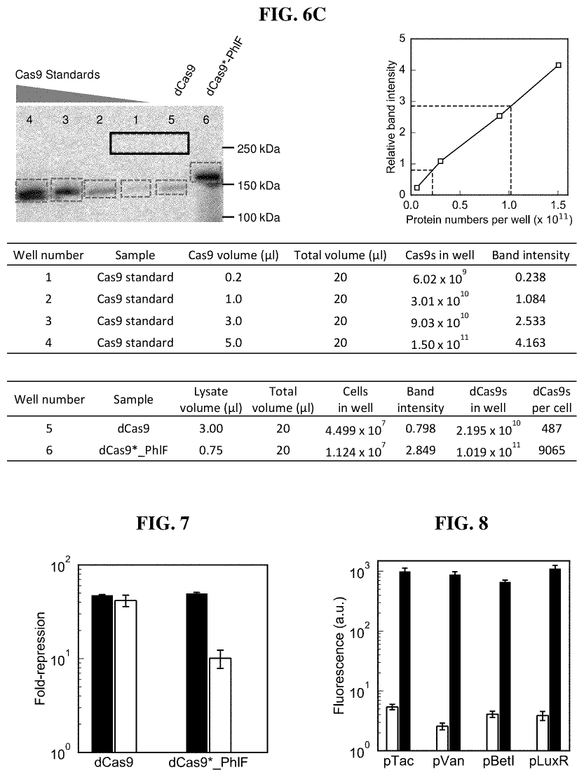

The same amount (10 μl) of the resulting standards and cell lysates were loaded on a 4-12% gradient SDS-PAGE gel (Lonza, #59524). After the run, the gels were transferred onto a PVDF membrane (Biorad, #162-0177) and then blocked at room temperature for 1 hr in 5% skim milk (w/v of TBST, 138 mM NaCl, 2.7 mM KCl, 0.1% Triton X-100, 25 mM Tris-HCl, pH 8.0). The anti-Cas9 antibody (abcam, # ab202580) was used as primary antibody and added 1:2000 into 2.5% skim milk (w/v of TBST). The primary antibody solution was then added to the PVDF membrane and allowed to bind for 1 hour at room temperature. The membrane was then washed three times with TBST. The secondary antibody, HRP-conjugated anti-mouse antibody (Sigma, # A8924), was added to 1:4000 and incubated for 1 hour at room temperature. After washing the membrane, chemiluminescence for HRP (Pierce, #32106) was used to develop the signal and detected using the Biorad chemidoc MP imaging system (Biorad, #170-8280). ImageJ 1.41 (NIH) was used to analyze the gel densitometry. The relative protein numbers of dCas9 in the strain was calculated from the standard curve and known concentrations of Cas9 standards (FIGS. 6A-6C).

Random sequence generation.

The random sequences are generated using the online Random DNA Sequence Generator (www.faculty.ucr.edu/˜mmaduro/random.htm) with GC content set to 50%.

sgRNA array.

Pairs of ssDNA oligonucleotides ≤200 nt long that encode the necessary genetic parts (promoter, sgRNA, terminator) were ordered from Integrated DNA Technologies (IDT). These oligos are annealed by PCR using KAPA HiFi MasterMix (KAPA Biosystems, #07958935001) and the resulting dsDNA modules were then assembled in a one-pot Golden Gate assembly reaction using type II enzymes BsaI (New England Biolabs, # R0535S) or BsmbI (New England Biolabs, # R0580S) to generate plasmids with different numbers of sgRNAs. After transformation, these plasmids were re-purified and digested with restriction enzyme BsphI (New England Biolabs, # R0517S) to make sure they have the expected sizes and thus rule out the possibility of unwanted homologous recombination during construction and transformation (FIG. 11).

Energy cost of expressing dCas9*_PhlF and TetR.

The tetR gene is 624 bp and the translated TetR protein contains 207 amino acids. Based on a previous study (Kaleta C., et al., Biotechnol. J., 2013 September; 8(9): 1105-14), for transcription, 0.6 ATP is needed per nucleotide triplet. The required ATPs for transcription of tetR mRNA would be: 0.6×624/3=124.8. In addition, the required ATPs to synthesize each amino acid from glucose were obtained from TABLE 1 of the same study (Kaleta C., et al., Biotechnol. J., 2013 September; 8(9): 1105-14), and the ATPs required for synthesizing amino acids in the TetR protein can be calculated, which is −307 (the negative value means net production of ATPs). For translation, 4 ATPs are needed per amino acid, and thus the ATPs required are 4×207=828. Overall, the ATPs required for synthesizing one TetR protein are: 124.8−307+828=645.8. The engineered dCas9*_PhlF protein contains 1511 amino acids (4536 bp DNA), and the ATPs required for each of these steps are: 907.2, −795, 6044. The overall ATPs consumption for synthesizing one dCas9*_PhlF protein would be 907.2−795+6044=6156.2.

Combining response functions.

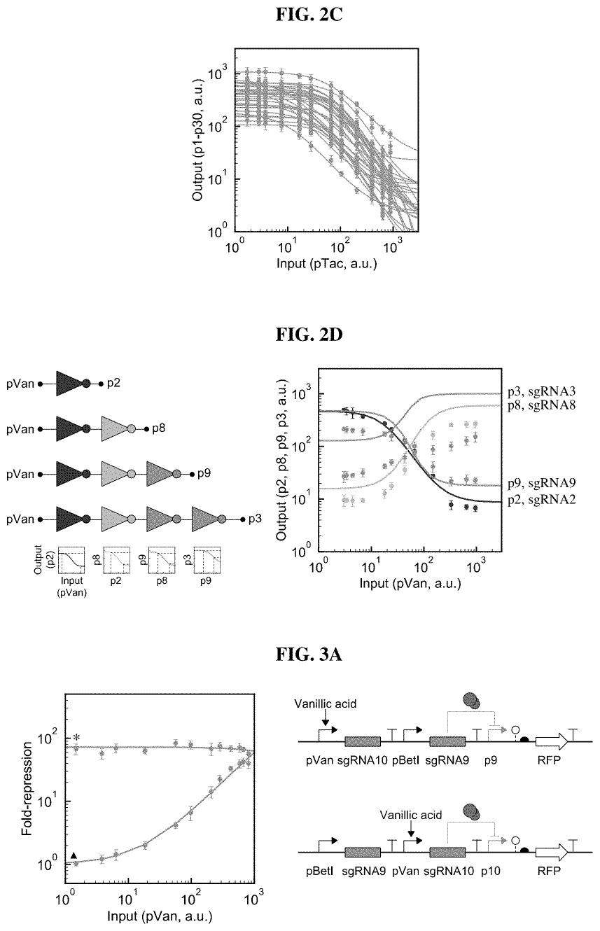

In the layered cascade circuit of NOT gates, the output values from the previous layer serve as the input values to the current layer (FIG. 2D). The pVan promoter was used as the input to the circuit, with measured ON (956 a.u.) and OFF (3 a.u.) fluorescence values. The corresponding OFF (9 a.u.) and ON (451 a.u.) values of promoter p2 were calculated from Equation 1, by using parameters of Gate2 (TABLE 1). The ON and OFF values from p2 promoter then served as inputs to the second NOT gate (Gate8). Next, ON and OFF values of promoter p8 were calculated from Equation 1 by using parameters of Gate8 (TABLE 1), which are 588 a.u. (ON) and 15 a.u. (OFF). The ON and OFF values for Gate9 and Gate3 were calculated following the same steps. For Gate9, the values are 470 a.u. (ON) and 18 a.u. (OFF). For Gate3, the values are 1002 a.u. (ON) and 129 a.u. (OFF).

TABLE 1
Measured gate parameters.

| Gate ^{b} | Yₘᵢₙ | Yₘₐₓ | K | n |
|---|---|---|---|---|
| Gate01 | 7.3 | 280 | 36 | 1.5 |
| Gate02 | 8.7 | 470 | 19 | 1.7 |
| Gate03 | 27 | 1100 | 88 | 1.4 |
| Gate04 | 5.8 | 670 | 42 | 1.3 |
| Gate05 | 6.4 | 510 | 26 | 1.5 |
| Gate06 | 3.6 | 200 | 47 | 1.5 |
| Gate07 | 2.8 | 170 | 23 | 1.6 |
| Gate08 | 9.8 | 740 | 21 | 1.6 |
| Gate09 | 10 | 710 | 25 | 1.4 |
| Gate10 | 10 | 609 | 49 | 1.5 |
| Gate11 | 5.3 | 260 | 34 | 1.6 |
| Gate12 | 23 | 500 | 42 | 1.6 |
| Gate13 | 1.4 | 230 | 80 | 1.8 |
| Gate14 | 1.9 | 420 | 85 | 1.8 |
| Gate15 | 0.9 | 150 | 59 | 1.8 |
| Gate16 | 1.2 | 340 | 82 | 1.7 |
| Gate17 | 1.4 | 320 | 27 | 1.4 |
| Gate18 | 0.9 | 450 | 103 | 1.7 |
| Gate19 | 1.5 | 590 | 77 | 1.7 |
| Gate20 | 2.5 | 670 | 68 | 1.5 |
| Gate21 | 0.2 | 130 | 63 | 1.7 |
| Gate22 | 0.4 | 160 | 56 | 1.7 |
| Gate23 | 0.3 | 370 | 72 | 1.7 |
| Gate24 | 2.1 | 450 | 89 | 1.7 |
| Gate25 | 2.3 | 410 | 73 | 1.8 |
| Gate26 | 1.2 | 500 | 78 | 1.7 |
| Gate27 | 2.0 | 250 | 64 | 1.5 |
| Gate28 | 0.9 | 570 | 56 | 1.5 |
| Gate29 | 5.8 | 670 | 104 | 1.7 |
| Gate30 | 2.2 | 110 | 63 | 1.7 |

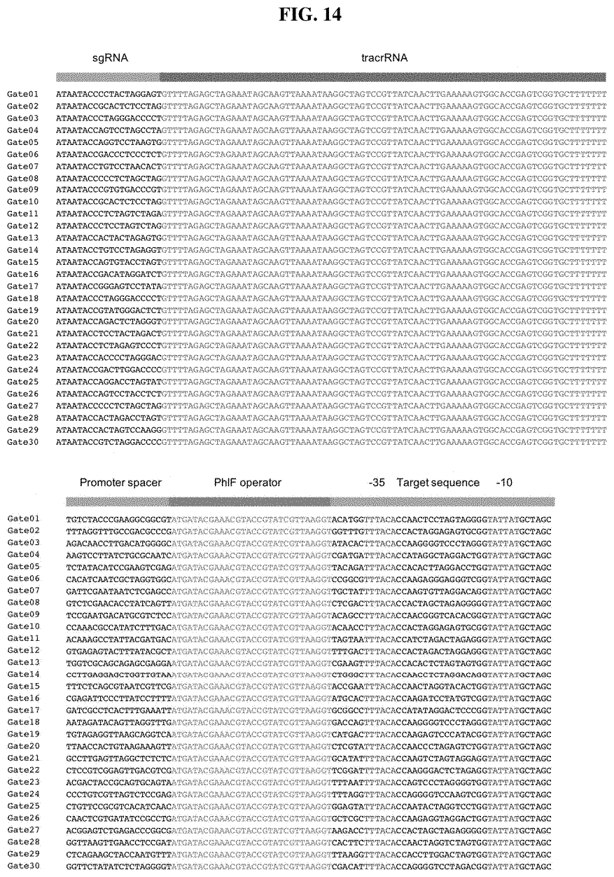

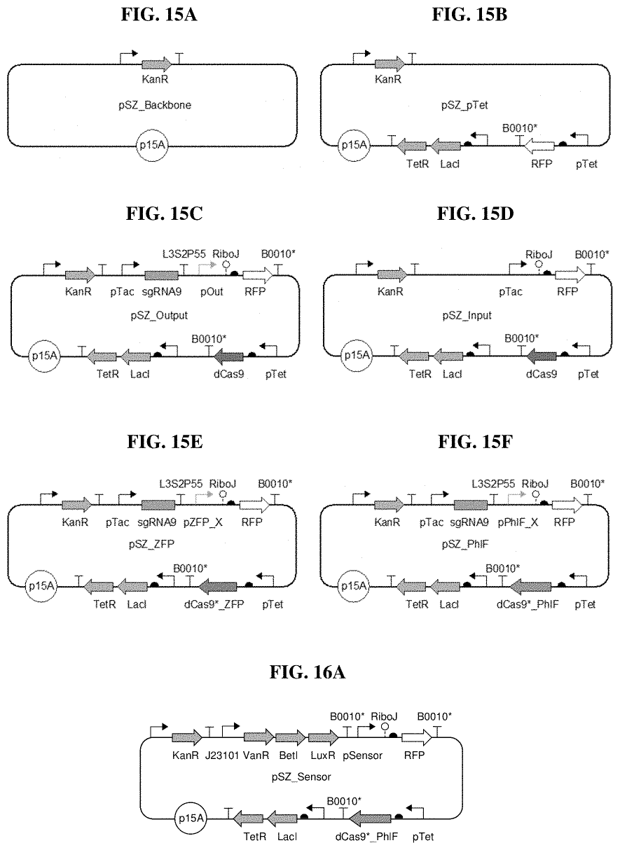

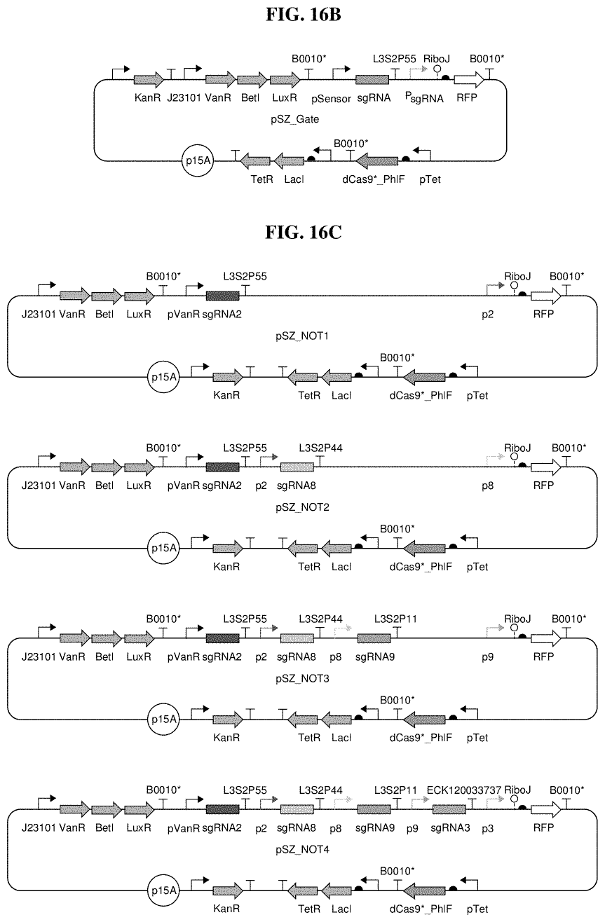

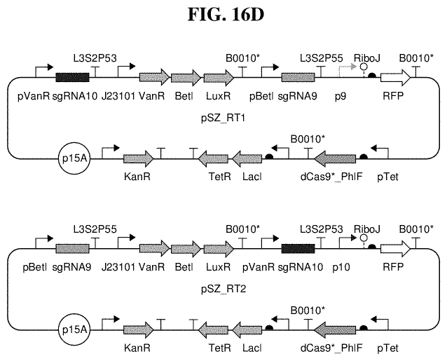

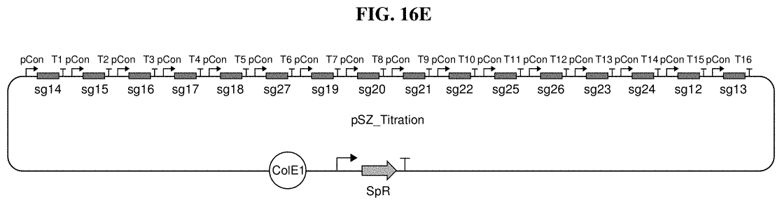

Parameters are shown for a fit to Equation 1 in main text. Gate sequences are provided in FIG. 14 and TABLE 2.

TABLE 2
Gate sequences.

| Gate | | | SEQ ID |
|---|---|---|---|
| Number | Part | DNA Sequence | NO |
| Gate01 | sgRNA- | ATAATACCCCTACTAGGAGTGTTTTAGAGCTAGAAATAG | 1 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | TGTCTACCCGAAGGCGGCGTATGATACGAAACGTACCGT | 2 |
| | PhIF operator- | ATCGTTAAGGTACATGGTTTACACCAACTCCTAGTAGGG | |
| | -35 Target | GTATTATGCTAGC | |
| | sequence -10 | | |
| Gate02 | sgRNA- | ATAATACCGCACTCTCCTAGGTTTTAGAGCTAGAAATAG | 3 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | TTTAGGTTTGCCGACGCCCGATGATACGAAACGTACCGT | 4 |
| | PhIF operator- | ATCGTTAAGGTGGTTTGTTTACACCACTAGGAGAGTGCG | |
| | -35 Target | GTATTATGCTAGC | |
| | sequence -10 | | |
| Gate03 | sgRNA- | ATAATACCCTAGGGACCCCTGTTTTAGAGCTAGAAATAG | 5 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | AGACAACCTTGACATGGGGCATGATACGAAACGTACCGT | 6 |
| | PhIF operator- | ATCGTTAAGGTATACACTTTACACCAAGGGGTCCCTAGG | |
| | -35 Target | GTATTATGCTAGC | |
| | sequence -10 | | |
| Gate04 | sgRNA- | ATAATACCAGTCCTAGCCTAGTTTTAGAGCTAGAAATAG | 7 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | AAGTCCTTATCTGCGCAATCATGATACGAAACGTACCGT | 8 |
| | PhIF operator- | ATCGTTAAGGTCGATGATTTACACCATAGGCTAGGACTG | |
| | -35 Target | GTATTATGCTAGC | |
| | sequence -10 | | |
| Gate05 | sgRNA- | ATAATACCAGGTCCTAAGTGGTTTTAGAGCTAGAAATAG | 9 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | TCTATACATCCGAAGTCGAGATGATACGAAACGTACCGT | 10 |
| | PhIF operator- | ATCGTTAAGGTTACAGATTTACACCACACTTAGGACCTG | |
| | -35 Target | GTATTATGCTAGC | |
| | sequence -10 | | |
| Gate06 | sgRNA- | ATAATACCGACCCTCCCTCTGTTTTAGAGCTAGAAATAG | 11 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | CACATCAATCGCTAGGTGGCATGATACGAAACGTACCGT | 12 |
| | PhIF operator- | ATCGTTAAGGTCCGGCGTTTACACCAAGAGGGAGGGTCG | |
| | -35 Target | GTATTATGCTAGC | |
| | sequence -10 | | |
| Gate07 | sgRNA- | ATAATACCTGTCCTAACACTGTTTTAGAGCTAGAAATAG | 13 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | GATTCGAATAATCTCGAGCCATGATACGAAACGTACCGT | 14 |
| | PhIF operator- | ATCGTTAAGGTTGCTATTTTACACCAAGTGTTAGGACAG | |
| | -35 Target | GTATTATGCTAGC | |
| | sequence -10 | | |
| Gate08 | sgRNA- | ATAATACCCCCTCTAGCTAGGTTTTAGAGCTAGAAATAG | 15 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | GTCTCGAACACCTATCAGTTATGATACGAAACGTACCGT | 16 |
| | PhIF operator- | ATCGTTAAGGTCTCGACTTTACACCACTAGCTAGAGGGG | |
| | -35 Target | GTATTATGCTAGC | |
| | sequence -10 | | |
| Gate09 | sgRNA- | ATAATACCCGTGTGACCCGTGTTTTAGAGCTAGAAATAG | 17 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | TCCGAATGACATGCGTCTCCATGATACGAAACGTACCGT | 18 |
| | PhIF operator- | ATCGTTAAGGTACAGCCTTTACACCAACGGGTCACACGG | |
| | -35 Target | GTATTATGCTAGC | |
| | sequence -10 | | |
| Gate10 | sgRNA- | ATAATACCGCACTCTCCTAGGTTTTAGAGCTAGAAATAG | 19 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | CCAAACGCCATATCTTTGACATGATACGAAACGTACCGT | 20 |
| | PhIF operator- | ATCGTTAAGGTACAACCTTTACACCACTAGGAGAGTGCG | |
| | -35 Target | GTATTATGCTAGC | |
| | sequence -10 | | |
| Gate11 | sgRNA- | ATAATACCCTCTAGTCTAGAGTTTTAGAGCTAGAAATAG | 21 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | ACAAAGCCTATTACGATGACATGATACGAAACGTACCGT | 22 |
| | PhIF operator- | ATCGTTAAGGTTAGTAATTTACACCATCTAGACTAGAGG | |
| | -35 Target | GTATTATGCTAGC | |
| | sequence -10 | | |
| Gate12 | sgRNA- | ATAATACCCTCCTAGTCTAGGTTTTAGAGCTAGAAATAG | 23 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | GTGAGAGTACTTTATACGCTATGATACGAAACGTACCGT | 24 |
| | PhIF operator- | ATCGTTAAGGTTTTGACTTTACACCACTAGACTAGGAGG | |
| | -35 Target | GTATTATGCTAGC | |
| | sequence -10 | | |
| Gate13 | sgRNA- | ATAATACCACTACTAGAGTGGTTTTAGAGCTAGAAATAG | 25 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | TGGTCGCAGCAGAGCGAGGAATGATACGAAACGTACCGT | 26 |
| | PhIF operator- | ATCGTTAAGGTCGAAGTTTTACACCACACTCTAGTAGTG | |
| | -35 Target | GTATTATGCTAGC | |
| | sequence -10 | | |
| Gate14 | sgRNA- | ATAATACCTGTCCTAGAGGTGTTTTAGAGCTAGAAATAG | 27 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | CCTTGAGGAGCTGGTTGTAAATGATACGAAACGTACCGT | 28 |
| | PhIF operator- | ATCGTTAAGGTCTGGGCTTTACACCAACCTCTAGGACAG | |
| | -35 Target | GTATTATGCTAGC | |
| | sequence -10 | | |
| Gate15 | sgRNA- | ATAATACCAGTGTACCTAGTGTTTTAGAGCTAGAAATAG | 29 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | TTTCTCAGCGTAATCGTTCGATGATACGAAACGTACCGT | 30 |
| | PhIF operator- | ATCGTTAAGGTACCGAATTTACACCAACTAGGTACACTG | |
| | -35 Target | GTATTATGCTAGC | |
| | sequence -10 | | |
| Gate16 | sgRNA- | ATAATACCGACATAGGATCTGTTTTAGAGCTAGAAATAG | 31 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | CGAGATTCCCTTATCCTTTTATGATACGAAACGTACCGT | 32 |
| | PhIF operator- | ATCGTTAAGGTATGCACTTTACACCAAGATCCTATGTCG | |
| | -35 Target | GTATTATGCTAGC | |
| | sequence -10 | | |
| Gate17 | sgRNA- | ATAATACCGGGAGTCCTATAGTTTTAGAGCTAGAAATAG | 33 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | GATCGCCTCACTTTGAAATTATGATACGAAACGTACCGT | 34 |
| | PhIF operator- | ATCGTTAAGGTGCGGCCTTTACACCATATAGGACTCCCG | |
| | -35 Target | GTATTATGCTAGC | |
| | sequence -10 | | |
| Gate18 | sgRNA- | ATAATACCCTAGGGACCCCTGTTTTAGAGCTAGAAATAG | 35 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | AATAGATACAGTTAGGTTTGATGATACGAAACGTACCGT | 36 |
| | PhIF operator- | ATCGTTAAGGTGACCAGTTTACACCAAGGGGTCCCTAGG | |
| | -35 Target | GTATTATGCTAGC | |
| | sequence -10 | | |
| Gate19 | sgRNA- | ATAATACCGTATGGGACTCTGTTTTAGAGCTAGAAATAG | 37 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | TGTAGAGGTTAAGCAGGTCAATGATACGAAACGTACCGT | 38 |
| | PhIF operator- | ATCGTTAAGGTCATGACTTTACACCAAGAGTCCCATACG | |
| | -35 Target | GTATTATGCTAGC | |
| | sequence -10 | | |
| Gate20 | sgRNA- | ATAATACCAGACTCTAGGGTGTTTTAGAGCTAGAAATAG | 39 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | TTAACCACTGTAAGAAAGTTATGATACGAAACGTACCGT | 40 |
| | PhIF operator- | ATCGTTAAGGTCTCGTATTTACACCAACCCTAGAGTCTG | |
| | -35 Target | GTATTATGCTAGC | |
| | sequence -10 | | |
| Gate21 | sgRNA- | ATAATACCTCCTACTAGACTGTTTTAGAGCTAGAAATAG | 41 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | GCCTTGAGTTAGGCTCTCTCATGATACGAAACGTACCGT | 42 |
| | PhIF operator- | ATCGTTAAGGTGCATATTTTACACCAAGTCTAGTAGGAG | |
| | -35 Target | GTATTATGCTAGC | |
| | sequence -10 | | |
| Gate22 | sgRNA- | ATAATACCTCTAGAGTCCCTGTTTTAGAGCTAGAAATAG | 43 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | CTCCGTCGGAGTTGACGTCGATGATACGAAACGTACCGT | 44 |
| | PhIF operator- | ATCGTTAAGGTTCGGATTTTACACCAAGGGACTCTAGAG | |
| | -35 Target | GTATTATGCTAGC | |
| | sequence -10 | | |
| Gate23 | sgRNA- | ATAATACCACCCCTAGGGACGTTTTAGAGCTAGAAATAG | 45 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | ACGACTACCGCAGTGCAGTAATGATACGAAACGTACCGT | 46 |
| | PhIF operator- | ATCGTTAAGGTTTTAATTTTACACCAGTCCCTAGGGGTG | |
| | -35 Target | GTATTATGCTAGC | |
| | sequence -10 | | |
| Gate24 | sgRNA- | ATAATACCGACTTGGACCCCGTTTTAGAGCTAGAAATAG | 47 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | CCCTGTCGTTAGTCTCCGAGATGATACGAAACGTACCGT | 48 |
| | PhIF operator- | ATCGTTAAGGTTTTAGGTTTACACCAGGGGTCCAAGTCG | |
| | -35 Target | GTATTATGCTAGC | |
| | sequence -10 | | |
| Gate25 | sgRNA- | ATAATACCAGGACCTAGTATGTTTTAGAGCTAGAAATAG | 49 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | CTGTTCCGCGTCACATCAACATGATACGAAACGTACCGT | 50 |
| | PhIF operator- | ATCGTTAAGGTGGAGTATTTACACCAATACTAGGTCCTG | |
| | -35 Target | GTATTATGCTAGC | |
| | sequence -10 | | |
| Gate26 | sgRNA- | ATAATACCAGTCCTACCTCTGTTTTAGAGCTAGAAATAG | 51 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | CAACTCGTGATATCCGCCTGATGATACGAAACGTACCGT | 52 |
| | PhIF operator- | ATCGTTAAGGTGCTCGCTTTACACCAAGAGGTAGGACTG | |
| | -35 Target | GTATTATGCTAGC | |
| | sequence -10 | | |
| Gate27 | sgRNA- | ATAATACCCCCTCTAGCTAGGTTTTAGAGCTAGAAATAG | 53 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | ACGGAGTCTGAGACCCGGCGATGATACGAAACGTACCGT | 54 |
| | PhIF operator- | ATCGTTAAGGTAAGACCTTTACACCACTAGCTAGAGGGG | |
| | -35 Target | GTATTATGCTAGC | |
| | sequence -10 | | |
| Gate28 | sgRNA- | ATAATACCACTAGACCTAGTGTTTTAGAGCTAGAAATAG | 55 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | GGTTAAGTTGAACCTCCGATATGATACGAAACGTACCGT | 56 |
| | PhIF operator- | ATCGTTAAGGTCACTTCTTTACACCAACTAGGTCTAGTG | |
| | -35 Target | GTATTATGCTAGC | |
| | sequence -10 | | |
| Gate29 | sgRNA- | ATAATACCACTAGTCCAAGGGTTTTAGAGCTAGAAATAG | 57 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | CTCAGAAGCTACCAATGTTTATGATACGAAACGTACCGT | 58 |
| | PhIF operator- | ATCGTTAAGGTTTAAGGTTTACACCACCTTGGACTAGTG | |
| | -35 Target | GTATTATGCTAGC | |
| | sequence -10 | | |
| Gate30 | sgRNA- | ATAATACCGTCTAGGACCCCGTTTTAGAGCTAGAAATAG | 59 |
| | tracrRNA | CAAGTTAAAATAAGGCTAGTCCGTTATCAACTTGAAAAA | |
| | | GTGGCACCGAGTCGGTGCTTTTTTT | |
| | Promoter spacer- | GGTTCTATATCTCTAGGGGTATGATACGAAACGTACCGT | 60 |
| | PhIF operator- | ATCGTTAAGGTCGACATTTTACACCAGGGGTCCTAGACG | |
| | -35 Target | GTATTATGCTAGC | |
| | sequence -10 | | |

Derivation for the impact of dCas9 sharing by multiple sgRNAs.

When multiple competing sgRNAs (i=2 . . . n) were expressed:

$$\begin{matrix}{C}_{TOT} = {C}_{F} + {C}_{s 1} + {\sum }_{i = 2}^{n} {C}_{si} , & ( 1 ) \\ \frac{{ds}_{1}}{d t} = {α}_{1} - {δ}_{s} {s}_{1} - {k}_{1} {C}_{F} {s}_{1} + {k}_{- 1} {C}_{s 1} , & ( 2 ) \\ \frac{{ds}_{i}}{d t} = {α}_{i} - {δ}_{s} {s}_{i} - {k}_{1} {C}_{F} {s}_{i} + {k}_{- 1} {C}_{s i} . & ( 3 )\end{matrix}$$

It was assumed that all of the co-expressed competing sgRNAs had the same transcription rate αᵢ=αₓ for i=2 . . . n.

For the formation of each sgRNA::dCas9 complex:

$$\begin{matrix}\frac{d {C}_{s 1}}{d t} = {k}_{1} {C}_{F} {s}_{1} - {k}_{- 1} {C}_{s 1} , & ( 4 ) \\ \frac{d {C}_{s i}}{d t} = {k}_{1} {C}_{F} {s}_{i} - {k}_{- 1} {C}_{s i} . & ( 5 )\end{matrix}$$

The dynamics of free dCas9 is given by:

$$\frac{d {C}_{F}}{dt} = - {k}_{1} {C}_{F} {s}_{1} - {\sum }_{i = 2}^{n} {k}_{1} {C}_{F} {s}_{i} + {k}_{- 1} {C}_{s 1} + {\sum }_{i = 2}^{n} {k}_{- 1} {C}_{s i} . \qquad ( 6 )$$

At steady-state, Equations 1-6 reduce to:

$$\begin{matrix}{s}_{1} = \frac{{α}_{1}}{{δ}_{s}} and {s}_{i} = \frac{{α}_{X}}{{δ}_{s}} , & ( 7 ) \\ {C}_{s 1} = \frac{{α}_{1} {C}_{TOT}}{β + {α}_{1} + N {α}_{X}} , where β = \frac{{δ}_{s}}{{K}_{1}} , & ( 8 )\end{matrix}$$

and N=n−1 is the number of co-expressed competing sgRNAs.

### Example 1. Experimental Design for Reducing Cas9 Toxicity

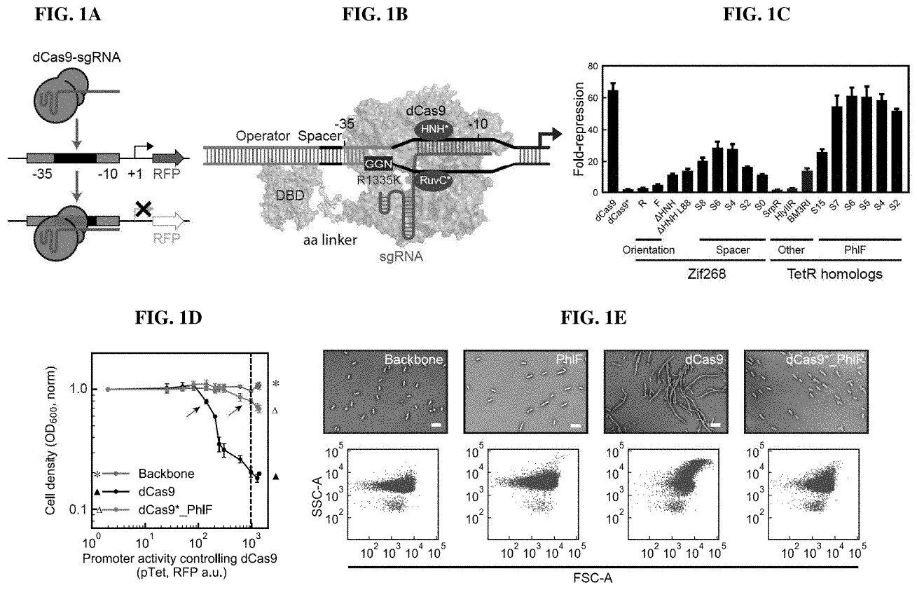

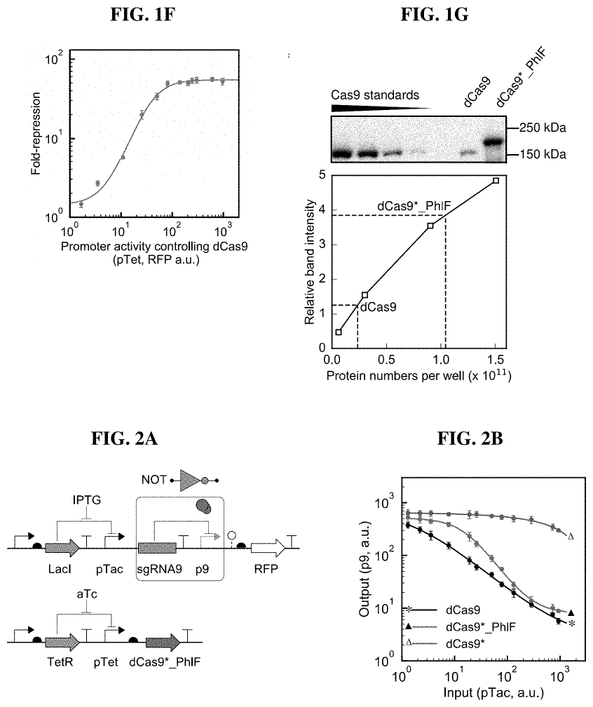

Transcription of a target reporter gene is blocked when a dCas9-sgRNA binds to its promoter (FIG. 1A). Following the hypothesis that non-specific dCas9 binding to DNA leads to its toxicity, a series of mutations intended to disrupt binding were made. A schematic of these modifications is shown in FIG. 1B. The RuvC* and HNH* domains are mutated to disrupt the nuclease activity of Cas9 to create dCas9 (Qi Lei S., et al., Cell, 2013. 152(5): 1173-83). The promoter is based on the strong constitutive promoter BBa_J23101 (Nielsen A. A. and Voigt C. A., Mol. Syst. Biol., 2014. 10(763): 1-11), modified upstream of the −10 position to contain a 20 bp sequence that is complementary to the cognate sgRNA. The activity of this promoter is measured using a transcriptional fusion to red fluorescent protein (rfp) and flow cytometry (Methods). Binding to the PAM site (NGG) is disrupted by making the R1335K mutation to dCas9 (Bolukbasi M. F., et al., Nat. Methods, 2015 December; 12(12): 1150-56). Various DNA-binding domains (DBD) are fused to the C-terminal end of dCas9 and the corresponding operator is placed upstream of the −35 promoter region, separated by a spacer. A linker is used to control the distance between the DBD and dCas9.

A reporter system was developed to evaluate the impact of these modifications on the ability for dCas9 to repress the targeted promoter (FIGS. 13A-13F). The expression of the sgRNA and dCas9 are controlled using IPTG- and aTc-inducible promoters, respectively. All of these components are integrated into a single p15A plasmid backbone. The fold-repression is measured as the fluorescence from the output promoter in the absence of sgRNA inducer (0 mM IPTG), divided by the fluorescence when the sgRNA is expressed (1 mM IPTG). When the R1335K mutation is made (dCas9*), this completely abolishes repression as expected (FIG. 1C).

### Example 2. Analyses of dCas9—ZFP Variant Activity

The ability for a zinc finger protein (ZFP) to recover nuclease activity was first tested. To this end, a variant of dCas9* described previously was built, where Zif268^{TS3} is fused to the C-terminal end of dCas9* via a 58 amino acid linker (Bolukbasi M. F., et al., Nat. Methods, 2015 December; 12(12): 1150-56). The corresponding 12 bp operator recognized by Zif268^{Ts3} was then placed upstream of the promoter, separated from the −35 position by a spacer (all promoter variants described are provided in TABLE 3). The orientation of the operator (forward and reverse) was initially tested with the forward yielding higher repression as previously observed (Bolukbasi M. F., et al., Nat. Methods, 2015 December; 12(12): 1150-56). Thus, it was selected for all subsequent optimization. The deletion of the nuclease domain (AHNH) (Sternberg S. H., et al., Nature, 2015. 527(7576): 110-13) and the increase in linker size to 88 amino acids (L88) both improved repression (FIG. 1C). Finally, the length of the spacer was varied between 0 to 8 bp and an optimum was identified at 6. Collectively, these changes resulted in a ZFP fused dCas9 that can only achieve a maximum of 28-fold repression, roughly a third of the activity of the unmodified variant.

### Example 3. Analyses of dCas9—TetR Variant Activity and Toxicity

TetR-family repressors were then evaluated in place of the ZFP using the same dCas9* variant (88 amino acid linker, AHNH). Four repressors were tested (PhlF, BM3RI, HlyIIR, and SrpR) and their corresponding operators (30 bp, 20 bp, 22 bp, 30 bp, TABLE 3) were inserted in front of the promoter with the 6 bp spacer (Stanton B. C., et al., Nat. Chem. Biol., 2014. 10(2): p. 99-105). Of these, the PhlF fusion (dCas9*_PhlF) recovered the most activity, achieving 95% of the repression of dCas9 with an optimal spacer length of 6 bp (FIG. 1C).

TABLE 3
Sequences of promoters used in FIG. 1C.

| Part | | | SEQ |
|---|---|---|---|
| name | Type | DNA Sequence | ID NO |
| pZFP_F | Promoter | TCCGAATGACATGCGTCTCCCGCTCCAACACCGTTGGTT | 61 |
| | | GAACAGCCTTTACACCAACGGGTCACACGGGTATTATGC | |
| | | TAGC | |
| pZFP_R | Promoter | TCCGAATGACATGCGTCTCCGGTGTTGGAGCGGTTGGTT | 62 |
| | | GAACAGCCTTTACACCAACGGGTCACACGGGTATTATGC | |
| | | TAGC | |
| pZFP_S0 | Promoter | TCCGAATGACATGCGTCTCCGGTGTTGGAGCGTTTACAC | 63 |
| | | CAACGGGTCACACGGGTATTATGCTAGC | |
| pZFP_S2 | Promoter | TCCGAATGACATGCGTCTCCGGTGTTGGAGCGCCTTTAC | 64 |
| | | ACCAACGGGTCACACGGGTATTATGCTAGC | |
| pZFP_S4 | Promoter | TCCGAATGACATGCGTCTCCGGTGTTGGAGCGAGCCTTT | 65 |
| | | ACACCAACGGGTCACACGGGTATTATGCTAGC | |
| pZFP_S6 | Promoter | TCCGAATGACATGCGTCTCCGGTGTTGGAGCGACAGCCT | 66 |
| | | TTACACCAACGGGTCACACGGGTATTATGCTAGC | |
| pZFP_S8 | Promoter | TCCGAATGACATGCGTCTCCGGTGTTGGAGCGGAACAGC | 67 |
| | | CTTTACACCAACGGGTCACACGGGTATTATGCTAGC | |
| pPhlF_S2 | Promoter | TCCGAATGACATGCGTCTCCATGATACGAAACGTACCGT | 68 |
| | | ATCGTTAAGGTCCTTTACACCAACGGGTCACACGGGTAT | |
| | | TATGCTAGC | |
| pPhlF_S4 | Promoter | TCCGAATGACATGCGTCTCCATGATACGAAACGTACCGT | 69 |
| | | ATCGTTAAGGTAGCCTTTACACCAACGGGTCACACGGGT | |
| | | ATTATGCTAGC | |
| pPhlF_S5 | Promoter | TCCGAATGACATGCGTCTCCATGATACGAAACGTACCGT | 70 |
| | | ATCGTTAAGGTCAGCCTTTACACCAACGGGTCACACGGG | |
| | | TATTATGCTAGC | |
| pPhlF_S6 | Promoter | TCCGAATGACATGCGTCTCCATGATACGAAACGTACCGT | 71 |
| | | ATCGTTAAGGTACAGCCTTTACACCAACGGGTCACACGG | |
| | | GTATTATGCTAGC | |
| pPhlF_S7 | Promoter | TCCGAATGACATGCGTCTCCATGATACGAAACGTACCGT | 72 |
| | | ATCGTTAAGGTAACAGCCTTTACACCAACGGGTCACACG | |
| | | GGTATTATGCTAGC | |
| pPhlF_S15 | Promoter | TCCGAATGACATGCGTCTCCATGATACGAAACGTACCGT | 73 |
| | | ATCGTTAAGGTGTTGGTTGAACAGCCTTTACACCAACGG | |
| | | GTCACACGGGTATTATGCTAGC | |
| pSrpR | Promoter | TCCGAATGACATGCGTCTCCATATACATACATGCTTGTT | 74 |
| | | TGTTTGTAAACACAGCCTTTACACCAACGGGTCACACGG | |
| | | GTATTATGCTAGC | |
| pHlyIIR | Promoter | TCCGAATGACATGCGTCTCCATATTTAAAATTCTTGTTT | 75 |
| | | AAAACAGCCTTTACACCAACGGGTCACACGGGTATTATG | |
| | | CTAGC | |
| pBM3RI | Promoter | TCCGAATGACATGCGTCTCCCGGAATGAACGTTCATTCC | 76 |
| | | GACAGCCTTTACACCAACGGGTCACACGGGTATTATGCT | |
| | | AGC | |

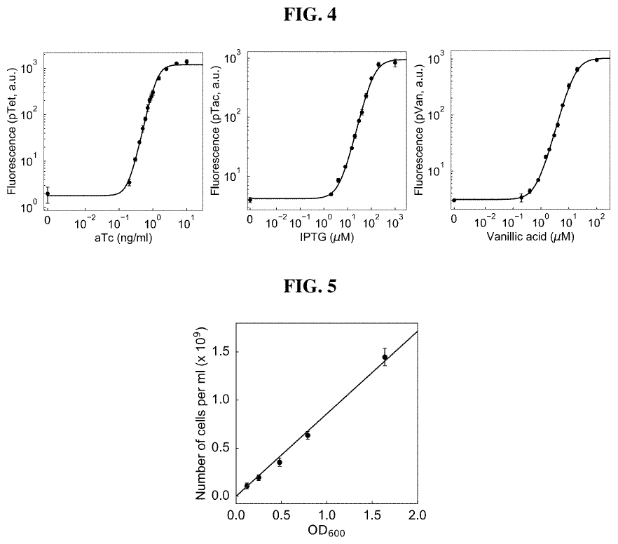

The growth impact of dCas9 was then compared to dCas9*_PhlF at different levels of expression, controlled by the addition of aTc. The activity of the pTet promoter is used as a surrogate of dCas9 expression, measured in independent experiments using a separate plasmid and red fluorescent protein (FIG. 4). There is a clear impact on growth, where cells expressing dCas9 rapidly declines past an expression threshold (FIG. 1D). In contrast, there is only a slight defect at the highest expression levels of dCas9*_PhlF. The morphological impact on the cell can be seen when aliquots are compared at the same level of inducer (2.5 ng/ml aTc) (FIG. 1E). The expression of dCas9* leads to longer cells and larger side scatter (SSC-A) (Tzur A., et al., PloS One, 2011 Jan. 20; 6(1): e16053), an effect described previously (Cho S., et al., ACS Synth. Biol., 2018 Apr. 20; 7(4): 1085-94). However, when expressing dCas9*_PhlF or PhlF alone, the same level of inducer leads to cell morphologies similar to wild-type E. coli. Next, whether the changes made to build dCas9*_PhlF simply disrupted its ability to act as a repressor was tested. Repression saturates at an expression level well before any growth defect is observed (FIG. 1F), thus indicating the changes are not impacting performance.

Note that the use of promoter strengths to compare expression levels between dCas9 and dCas9*_PhlF is, at best, inexact as these genes will translate differently. Therefore, immunoblotting was performed to quantify the size of the pools of each protein that the cell can tolerate before a growth impact is observed. Based on the growth experiment, 0.7 ng/ml aTc was chosen for dCas9 and 2.5 ng/ml aTc for dCas9*_PhlF as the inducer levels just prior to the corresponding thresholds (arrows in FIG. 1D). The details of these experiments are presented in the Methods. Briefly, a standard curve was generated using commercially-available Cas9 of known concentration and a Cas9-targeting monoclonal antibody (FIG. 1G). Then, wells are loaded with whole cell lysate from strains expressing dCas9 or dCas9*_PhlF and the dCas9 number per well can be calculated from band intensity of that well by comparing to the standard curve. The number of cells per ml were also measured and used in the calculation (FIG. 5). The average of three biological replicates, one of which is shown in FIG. 1G, determined that 9600±800 molecules of dCas9*_PhlF and 530±40 molecules of dCas9* are tolerated by a cell before growth and morphology defects are observed (FIGS. 6A-6C).

### Example 4. Design and Characterization of sgRNA-Promoter Pairs as NOT Gates

A transcriptional NOT gate inverts the response of a promoter (Yokobayashi Y., et al., Proc. Natl. Acad. Sci. USA, 2002 Dec. 24; 99(26): 16587-91). More complex circuits can be constructed by connecting NOT gates to each other (e.g., toggle switch and oscillator) or by converting to NOR gates through the addition of a second upstream input promoter (Nielsen A. A., et al., Science, 2016. 352(6281): aac7341; Gardner T. S., et al., Nature, 2000 Jan. 20; 403(6767): 339-42; Elowitz M. B. and Leibler S., Nature, 2000. 403(6767): 335-38; Tamsir A., et al., Nature, 2011. 469(7329): 212-15). Previously, an architecture was designed for NOT and NOR gates based on sgRNAs using dCas9 (Nielsen A. A. and Voigt C. A., Mol. Syst. Biol., 2014. 10(763): 1-11). Here, this approach was followed to build gates based on dCas9*_PhlF, where the input promoter driving sgRNA is an IPTG-inducible pTac promoter (FIG. 2A). The response of the output promoter is measured using a transcriptional fusion to rfp. These were combined to build a single plasmid using the p15A backbone (FIGS. 13A-13F). The plasmid was transformed into E. coli and cells were grown in inducer until reaching steady-state (Methods).

The response function is characterized by comparing the activity of the pTac promoter, measured separately, versus the activity of the output promoter (FIG. 2B and Methods). The resulting data can be fit to the equation,

$$y = {Y}_{min} + ( {Y}_{max} - {Y}_{min} ) \frac{{K}^{n}}{{x}^{n} + {K}^{n}} , \qquad ( 1 )$$

where y is the output promoter activity (and Yₘₐₓ/Yₘᵢₙ are the maximum/minimum activities), x is the input promoter activity, K is the threshold and n is the cooperativity. Note that the values of the promoter activities are in arbitrary units of red fluorescence and not standardized units. The response function from dCas9 is linear over the entire range of input with n=0.9, as observed previously (FIG. 2B). However, the response function resulting from dCas9*_PhlF has a clear S-shape with n=1.6. The increased cooperativity could be due to the multimerization of PhlF, a mechanism supported by the loss in repression observed by adding the PhlF inducer DAPG (FIG. 7).

A library of NOT gates was then built based on a set of 30 orthogonal sgRNAs (Nielsen A. A. and Voigt C. A., Mol. Syst. Biol., 2014. 10(763): 1-11). The target sequence corresponding to each was used to construct a promoter based on the system shown in FIG. 1B. The resulting NOT gates were then characterized as before and fit to Equation 1. The shapes of the curves are similar, but the maximum activity shifts as a result of the operator changes impacting promoter strength (FIG. 2C). On average, the gates exhibit a 47-fold dynamic range and the cooperativities span from 1.3 to 1.8. Because there are no cross reactions between gates, these could be used as the basis for the construction of large genetic circuits.

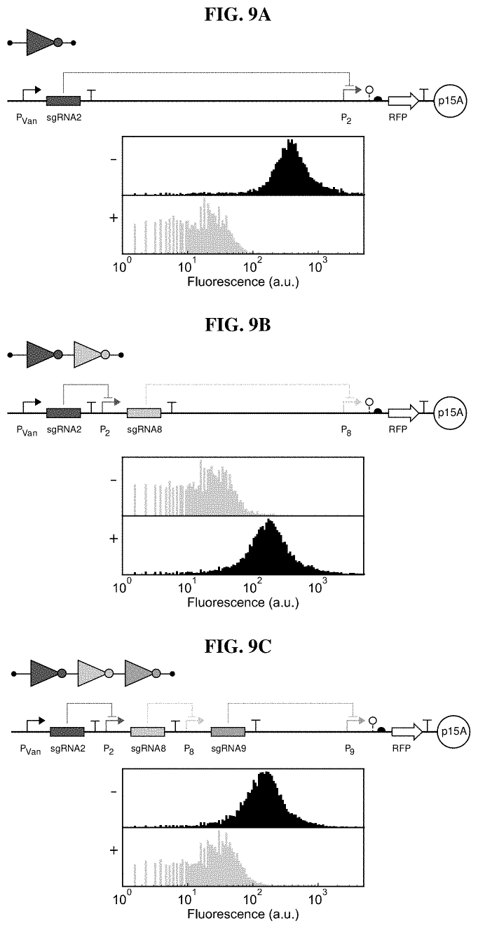

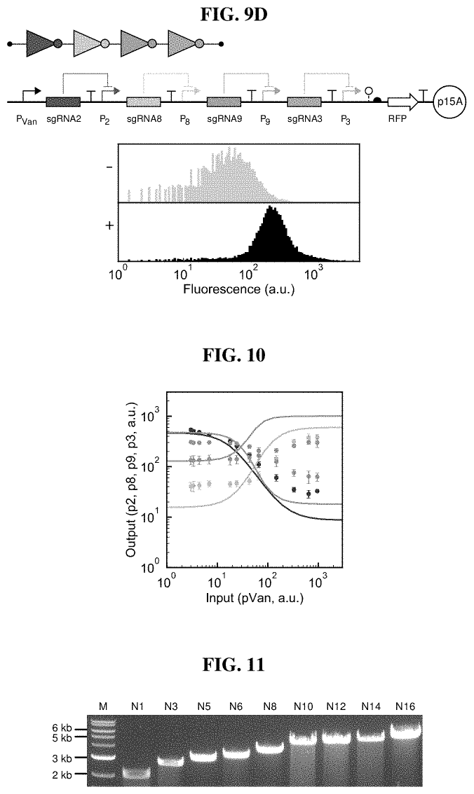

Cascades were constructed to demonstrate the layering of gates. First, the vanillic acid inducible system (pVan) was selected to serve as the input because it was observed to generate the largest dynamic range (341-fold) (FIG. 8). This was used as the input for a series of cascades based on 1 to 4 sgRNAs (FIG. 2D and FIGS. 9A-9C). The predicted response (solid lines) of each cascade was calculated by mathematically combining the response functions of the individually-measured gates (Methods). For the first three layers, the measured response closely matches that predicted. However, the addition of the fourth layer leads to a significant deviation from the predicted response. When dCas9*PhlF was expressed at lower levels, the measured responses deviated from the predicted responses even in the first two layers (FIG. 10).

### Example 5. Sharing of the dCas9*_PhlF Pool by Multiple sgRNAs

Genetic circuits with more than one gate require the simultaneous expression of multiple sgRNAs within the cell that need to compete with the same pool of dCas9 molecules. The sharing impacts the dynamics of each component in the system and this can have unintended consequences for the overall behavior of the circuit (Del Vecchio D., et al., Mol. Syst. Biol., 2008. 4(161): 1-16). Therefore, it is important to quantify the titration that occurs as more sgRNAs are simultaneously expressed.

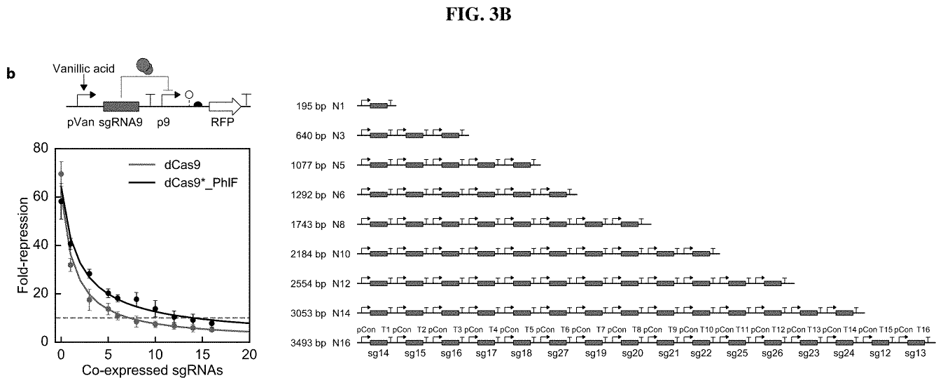

First, the impact of resource sharing between two sgRNAs was characterized (FIG. 3A). The pBetI promoter was used to generate a constitutive level of sgRNA9, which represses the p9 promoter. The vanillic acid inducible promoter (pVan) then drives a second sgRNA10. As vanillic acid is added and the second sgRNA is transcribed at higher levels, there is almost no impact on the ability of the first to repress its promoter. This is true even when sgRNA10 is expressed at the level required for the full repression of its cognate p10 promoter. Therefore, both sgRNAs can be fully expressed and independently repress two promoters without incurring significant effects due to resource sharing.

It is expected that as more sgRNAs are added to the system, at some point there would be a decline in their ability to function as dCas9*_PhlF is titrated. To quantify this transition, a mathematical model was developed inspired closely by the work of Del Vecchio and co-workers (Chen P. Y., et al., bioRxiv, 2018 Feb. 4: doi.org/10.1101/266015). The equations corresponding to when two sgRNAs are expressed are described below and this is expanded to a system of i sgRNAs in the Methods section. The pool of total dCas9 C_{TOT} is assumed to be constant. It can be described as the algebraic sum of free dCas9 C_{F} and the concentrations of dCas9 bound to the first and second sgRNAs (s₁ and s₂),

C_{TOT}=C_{F}+Cₛ₁+Cₛ₂ (2)

The dynamics of the unbound sgRNAs s₁ and s₂ are captured by the differential equations

$$\begin{matrix}\frac{{ds}_{1}}{d t} = {α}_{1} - {δ}_{s} {s}_{1} - {k}_{1} {C}_{F} {s}_{1} + {k}_{- 1} {C}_{s 1} and & ( 3 ) \\ \frac{{ds}_{2}}{d t} = {α}_{2} - {δ}_{s} {s}_{2} - {k}_{1} {C}_{F} {s}_{2} + {k}_{- 1} {C}_{s 2} and & ( 4 )\end{matrix}$$

where α₁ and α₂ are the transcription rates of the first and second sgRNAs. δₛ is degradation rates, and assumed to be the same for different sgRNAs. Similarly, the on- and off-rates of sgRNAs to dCas9 (k₁ and k₋₁) are assumed to be sequence independent. There are two additional differential equations for the formation of sgRNA::dCas9 complexes:

$$\begin{matrix}\frac{d {C}_{s 1}}{d t} = {k}_{1} {C}_{F} {s}_{1} - {k}_{- 1} {C}_{s 1} and & ( 5 ) \\ \frac{d {C}_{s 2}}{d t} = {k}_{1} {C}_{F} {s}_{2} - {k}_{- 1} {C}_{s 2} . & ( 6 )\end{matrix}$$

Finally, the concentration of free dCas9 is given by

$$\frac{d {C}_{F}}{dt} = - {k}_{1} {C}_{F} {s}_{1} - {k}_{1} {C}_{F} {s}_{2} + {k}_{- 1} {C}_{s 1} + {k}_{- 1} {C}_{s 2} . \qquad ( 7 )$$

At steady-state, Equations 1-6 reduce to

$$\begin{matrix}{s}_{1} = \frac{{α}_{1}}{{δ}_{s}} and & ( 8 ) \\ {C}_{s 1} = \frac{{K}_{1} {s}_{1} {C}_{TOT}}{1 + {K}_{1} {s}_{1} + {K}_{1} {s}_{2}} , & ( 9 )\end{matrix}$$

where K₁ is the association equilibrium constant of sgRNA to dCas9. This captures how increasing the concentration of the second sgRNA impacts the concentration of complexes with the first. By substituting sgRNA concentration from Equation 9, one can simplify Equation 9 to

$${C}_{s 1} = \frac{{α}_{1} {C}_{TOT}}{β + {α}_{1} + {α}_{2}} , where β = \frac{{δ}_{s}}{{K}_{1}} . \qquad ( 10 )$$

Considering a Shea-Acker's model of a repressor binding to a promoter (related in form to Equation 1 of Example 4), the impact on transcription would be:

$$\frac{G}{{G}_{ss}} = 1 + \frac{{C}_{s 1}^{n}}{{K}^{n}} , \qquad ( 11 )$$

where G/Gₛₛ is the fold-repression, K is the dissociation equilibrium constant for dCas9::sgRNA binding to the promoter, and n is the cooperativity. Combining Equations 10 and 11 shows how the expression of a second sgRNA impact the repression of promoter responsive to the first sgRNA.

Similarly, concentration of the first sgRNA::dCas9 complex can be derived when multiple competing sgRNAs are co-expressed and sharing the dCas9 pool (Methods section):

$${C}_{s 1} = \frac{{α}_{1} {C}_{TOT}}{β + {α}_{1} + N {α}_{X}} . \qquad ( 12 )$$

where N is the number of additional co-expressed sgRNAs and αₓ is the transcription rate of these competing sgRNAs. The concentration for each of these competing sgRNAs is assumed to be equal. The fold-repression is calculated by substituting Cₛ₁ from Equation 12 into Equation 11.

To parameterize the model, how the response of a sgRNA declines as more competing sgRNAs are added to the system was measured. The response of a vanillic acid-driven NOT gate based on sgRNA9 was measured; alone, it generates 58-fold repression (FIG. 3B). Then, a series of constructs were designed to express increasing numbers of sgRNAs, from 1 to 16. Each expression unit consists of the same pCon constitutive promoter, a different sgRNA (but conserving the tracrRNA sequence), and different strong terminators (part sequences in TABLE 4). The constructs involve the repetition of these units within a single construct. While effort was made to minimize repetitive DNA, sufficient regions of sequence similarity remain so special cloning procedures were used and construct stability confirmed by digestion (Methods and FIG. 11).

TABLE 4
Sequences of genetic parts used in this study.

| Part | | | SEQ |
|---|---|---|---|
| name | Type | DNA Sequence | ID NO |
| J23101 | Promoter | TTTACAGCTAGCTCAGTCCTAGGTATTATGCTAGC | 77 |
| pCon | Promoter | TTTACACCAACTCCTAGTAGGGGTATTATGCTAGC | 78 |
| pTac | Promoter | TGTTGACAATTAATCATCGGCTCGTATAATGTGTGGAATTGTG | 79 |
| | | AGCGCTCACAATT | |
| pVan | Promoter | ATTGGATCCAATTGACAGCTAGCTCAGTCCTAGGTACCATTG | 80 |
| | | GATCCAAT | |
| pBetI | Promoter | AGCGCGGGTGAGAGGGATTCGTTACCAATAGACAATTGATTG | 81 |
| | | GACGTTCAATATAATGCTAGC | |
| pLuxR | Promoter | ACCTGTAGGATCGTACAGGTTTACGCAAGAAAATGGTTTGTT | 82 |
| | | ACAGTCGAATAAA | |
| pTet | Promoter | TACTCCACCGTTGGCTTTTTTCCCTATCAGTGATAGAGATTGA | 83 |
| | | CATCCCTATCAGTGATAGAGATAATGAGCAC | |
| N1 | sgRNA | TTTACACCAACTCCTAGTAGGGGTATTATGCTAGCATAATACC | 84 |
| | array | GCACTCTCCTAGGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTCTCGGTACCAAATTTTCGAAAAAAGACGCTGA | |
| | | AAAGCGTCTTTTTTCGTTTTGGTCC | |
| N3 | sgRNA | TTTACACCAACTCCTAGTAGGGGTATTATGCTAGCATAATACC | 85 |
| | array | GCACTCTCCTAGGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTCTCGGTACCAAATTTTCGAAAAAAGACGCTGA | |
| | | AAAGCGTCTTTTTTCGTTTTGGTCCccaaacgccatatctttgacTCCGTT | |
| | | AACGGTCACGAGTTTTTACACCAACTCCTAGTAGGGGTATTA | |
| | | TGCTAGCATAATACCTGTCCTAGAGGTGTTTTAGAGCTAGAA | |
| | | ATAGCAAGTTAAAATAAGGCTAGTCCGTTATCAACTTGAAAA | |
| | | AGTGGCACCGAGTCGGTGCTTTTTTTAAAAAAAAAAAAGGCC | |
| | | TCCCAAATCGGGGGGCCTTTTTTATTGATAACAAAAccttgaggag | |
| | | ctggttgtaaTTTACACCAACTCCTAGTAGGGGTATTATGCTAGCAT | |
| | | AATACCAGTGTACCTAGTGTTTTAGAGCTAGAAATAGCAAGT | |
| | | TAAAATAAGGCTAGTCCGTTATCAACTTGAAAAAGTGGCACC | |
| | | GAGTCGGTGCTTTTTTTCTCGGTACCAAATTCCAGAAAAGAG | |
| | | ACGCTTAACAGCGTCTTTTTTCGTTTTGGTCC | |
| N5 | sgRNA | TTTACACCAACTCCTAGTAGGGGTATTATGCTAGCATAATACC | 86 |
| | array | GCACTCTCCTAGGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTCTCGGTACCAAATTTTCGAAAAAAGACGCTGA | |
| | | AAAGCGTCTTTTTTCGTTTTGGTCCccaaacgccatatctttgacTCCGTT | |
| | | AACGGTCACGAGTTTTTACACCAACTCCTAGTAGGGGTATTA | |
| | | TGCTAGCATAATACCTGTCCTAGAGGTGTTTTAGAGCTAGAA | |
| | | ATAGCAAGTTAAAATAAGGCTAGTCCGTTATCAACTTGAAAA | |
| | | AGTGGCACCGAGTCGGTGCTTTTTTTAAAAAAAAAAAAGGCC | |
| | | TCCCAAATCGGGGGGCCTTTTTTATTGATAACAAAAccttgaggag | |
| | | ctggttgtaaTTTACACCAACTCCTAGTAGGGGTATTATGCTAGCAT | |
| | | AATACCAGTGTACCTAGTGTTTTAGAGCTAGAAATAGCAAGT | |
| | | TAAAATAAGGCTAGTCCGTTATCAACTTGAAAAAGTGGCACC | |
| | | GAGTCGGTGCTTTTTTTCTCGGTACCAAATTCCAGAAAAGAG | |
| | | ACGCTTAACAGCGTCTTTTTTCGTTTTGGTCCtttctcagcgtaatcgttcg | |
| | | CGAAATCGAAGGTGAAGGTGTTTACACCAACTCCTAGTAGGG | |
| | | GTATTATGCTAGCATAATACCGACATAGGATCTGTTTTAGAG | |
| | | CTAGAAATAGCAAGTTAAAATAAGGCTAGTCCGTTATCAACT | |
| | | TGAAAAAGTGGCACCGAGTCGGTGCTTTTTTTCCAATTATTGA | |
| | | AGGCCGCTAACGCGGCCTTTTTTTGTTTCTGGTCTCCCcgagattcc | |
| | | cttatccttttTTTACACCAACTCCTAGTAGGGGTATTATGCTAGCAT | |
| | | AATACCGGGAGTCCTATAGTTTTAGAGCTAGAAATAGCAAGT | |
| | | TAAAATAAGGCTAGTCCGTTATCAACTTGAAAAAGTGGCACC | |
| | | GAGTCGGTGCTTTTTTTCCAATTATTGAAGGCCTCCCAAATCG | |
| | | GGGGGCCTTTTTTATTGATAACAAAA | |
| N6 | sgRNA | TTTACACCAACTCCTAGTAGGGGTATTATGCTAGCATAATACC | 87 |
| | array | TGTCCTAGAGGTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTAAAAAAAAAAAAGGCCTCCCAAATCGGGGGG | |
| | | CCTTTTTTATTGATAACAAAAccttgaggagctggttgtaaTTTACACCAA | |
| | | CTCCTAGTAGGGGTATTATGCTAGCATAATACCAGTGTACCT | |
| | | AGTGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCTCGGTACCAAATTCCAGAAAAGAGACGCTTAACAGCGTCT | |
| | | TTTTTCGTTTTGGTCCtttctcagcgtaatcgttcgCGAAATCGAAGGTGA | |
| | | AGGTGTTTACACCAACTCCTAGTAGGGGTATTATGCTAGCAT | |
| | | AATACCGACATAGGATCTGTTTTAGAGCTAGAAATAGCAAGT | |
| | | TAAAATAAGGCTAGTCCGTTATCAACTTGAAAAAGTGGCACC | |
| | | GAGTCGGTGCTTTTTTTCCAATTATTGAAGGCCGCTAACGCGG | |
| | | CCTTTTTTTGTTTCTGGTCTCCCcgagattcccttatccttttTTTACACCAA | |
| | | CTCCTAGTAGGGGTATTATGCTAGCATAATACCGGGAGTCCT | |
| | | ATAGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCCAATTATTGAAGGCCTCCCAAATCGGGGGGCCTTTTTTATT | |
| | | GATAACAAAAgatcgcctcactttgaaattTATCAAAGAGTTCATGCGTTT | |
| | | TTACACCAACTCCTAGTAGGGGTATTATGCTAGCATAATACC | |
| | | CTAGGGACCCCTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTCTCGGTACCAAAAAAAAAAAAAAAGACGCTG | |
| | | AAAAGCGTCTTTTTTCGTTTTGGTCCaatagatacagttaggtttgTTTAC | |
| | | ACCAACTCCTAGTAGGGGTATTATGCTAGCATAATACCCCCT | |
| | | CTAGCTAGGTTTTAGAGCTAGAAATAGCAAGTTAAAATAAGG | |
| | | CTAGTCCGTTATCAACTTGAAAAAGTGGCACCGAGTCGGTGC | |
| | | TTTTTTTCTCGGTACCAAAAAAAAAAAAAAAGACGCTGAAAA | |
| | | GCGTCTTTTTTTTTTTTGGTCC | |
| N8 | sgRNA | TTTACACCAACTCCTAGTAGGGGTATTATGCTAGCATAATACC | 88 |
| | array | TGTCCTAGAGGTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTAAAAAAAAAAAAGGCCTCCCAAATCGGGGGG | |
| | | CCTTTTTTATTGATAACAAAAccttgaggagctggttgtaaTTTACACCAA | |
| | | CTCCTAGTAGGGGTATTATGCTAGCATAATACCAGTGTACCT | |
| | | AGTGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCTCGGTACCAAATTCCAGAAAAGAGACGCTTAACAGCGTCT | |
| | | TTTTTCGTTTTGGTCCtttctcagcgtaatcgttcgCGAAATCGAAGGTGA | |
| | | AGGTGTTTACACCAACTCCTAGTAGGGGTATTATGCTAGCAT | |
| | | AATACCGACATAGGATCTGTTTTAGAGCTAGAAATAGCAAGT | |
| | | TAAAATAAGGCTAGTCCGTTATCAACTTGAAAAAGTGGCACC | |
| | | GAGTCGGTGCTTTTTTTCCAATTATTGAAGGCCGCTAACGCGG | |
| | | CCTTTTTTTGTTTCTGGTCTCCCcgagattcccttatccttttTTTACACCAA | |
| | | CTCCTAGTAGGGGTATTATGCTAGCATAATACCGGGAGTCCT | |
| | | ATAGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCCAATTATTGAAGGCCTCCCAAATCGGGGGGCCTTTTTTATT | |
| | | GATAACAAAAgatcgcctcactttgaaattTATCAAAGAGTTCATGCGTTT | |
| | | TTACACCAACTCCTAGTAGGGGTATTATGCTAGCATAATACC | |
| | | CTAGGGACCCCTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTCTCGGTACCAAAAAAAAAAAAAAAGACGCTG | |
| | | AAAAGCGTCTTTTTTCGTTTTGGTCCaatagatacagttaggtttgTTTAC | |
| | | ACCAACTCCTAGTAGGGGTATTATGCTAGCATAATACCCCCT | |
| | | CTAGCTAGGTTTTAGAGCTAGAAATAGCAAGTTAAAATAAGG | |
| | | CTAGTCCGTTATCAACTTGAAAAAGTGGCACCGAGTCGGTGC | |
| | | TTTTTTTCTCGGTACCAAAAAAAAAAAAAAAGACGCTGAAAA | |
| | | GCGTCTTTTTTTTTTTTGGTCCacggagtctgagacTcggcgAAGGTCGT | |
| | | CCGTACGAAGGTTTTACACCAACTCCTAGTAGGGGTATTATG | |
| | | CTAGCATAATACCGTATGGGACTCTGTTTTAGAGCTAGAAAT | |
| | | AGCAAGTTAAAATAAGGCTAGTCCGTTATCAACTTGAAAAAG | |
| | | TGGCACCGAGTCGGTGCTTTTTTTCTCGGTACCAAACCAATTA | |
| | | TTGAAGACGCTGAAAAGCGTCTTTTTTCGTTTTGGTCCtgtagagg | |
| | | ttaagcaggtcaTTTACACCAACTCCTAGTAGGGGTATTATGCTAGC | |
| | | ATAATACCAGACTCTAGGGTGTTTTAGAGCTAGAAATAGCAA | |
| | | GTTAAAATAAGGCTAGTCCGTTATCAACTTGAAAAAGTGGCA | |
| | | CCGAGTCGGTGCTTTTTTTCTCGGTACCAAATTCCAGAAAAGA | |
| | | GACGCTTTTAGAGCGTCTTTTTTCGTTTTGGTCC | |
| N10 | sgRNA | TTTACACCAACTCCTAGTAGGGGTATTATGCTAGCATAATACC | 89 |
| | array | TGTCCTAGAGGTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTAAAAAAAAAAAAGGCCTCCCAAATCGGGGGG | |
| | | CCTTTTTTATTGATAACAAAAccttgaggagctggttgtaaTTTACACCAA | |
| | | CTCCTAGTAGGGGTATTATGCTAGCATAATACCAGTGTACCT | |
| | | AGTGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCTCGGTACCAAATTCCAGAAAAGAGACGCTTAACAGCGTCT | |
| | | TTTTTCGTTTTGGTCCtttctcagcgtaatcgttcgCGAAATCGAAGGTGA | |
| | | AGGTGTTTACACCAACTCCTAGTAGGGGTATTATGCTAGCAT | |
| | | AATACCGACATAGGATCTGTTTTAGAGCTAGAAATAGCAAGT | |
| | | TAAAATAAGGCTAGTCCGTTATCAACTTGAAAAAGTGGCACC | |
| | | GAGTCGGTGCTTTTTTTCCAATTATTGAAGGCCGCTAACGCGG | |
| | | CCTTTTTTTGTTTCTGGTCTCCCcgagattcccttatccttttTTTACACCAA | |
| | | CTCCTAGTAGGGGTATTATGCTAGCATAATACCGGGAGTCCT | |
| | | ATAGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCCAATTATTGAAGGCCTCCCAAATCGGGGGGCCTTTTTTATT | |
| | | GATAACAAAAgatcgcctcactttgaaattTATCAAAGAGTTCATGCGTTT | |
| | | TTACACCAACTCCTAGTAGGGGTATTATGCTAGCATAATACC | |
| | | CTAGGGACCCCTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTCTCGGTACCAAAAAAAAAAAAAAAGACGCTG | |
| | | AAAAGCGTCTTTTTTCGTTTTGGTCCaatagatacagttaggtttgTTTAC | |
| | | ACCAACTCCTAGTAGGGGTATTATGCTAGCATAATACCCCCT | |
| | | CTAGCTAGGTTTTAGAGCTAGAAATAGCAAGTTAAAATAAGG | |
| | | CTAGTCCGTTATCAACTTGAAAAAGTGGCACCGAGTCGGTGC | |
| | | TTTTTTTCTCGGTACCAAAAAAAAAAAAAAAGACGCTGAAAA | |
| | | GCGTCTTTTTTTTTTTTGGTCCacggagtctgagacTcggcgAAGGTCGT | |
| | | CCGTACGAAGGTTTTACACCAACTCCTAGTAGGGGTATTATG | |
| | | CTAGCATAATACCGTATGGGACTCTGTTTTAGAGCTAGAAAT | |
| | | AGCAAGTTAAAATAAGGCTAGTCCGTTATCAACTTGAAAAAG | |
| | | TGGCACCGAGTCGGTGCTTTTTTTCTCGGTACCAAACCAATTA | |
| | | TTGAAGACGCTGAAAAGCGTCTTTTTTCGTTTTGGTCCtgtagagg | |
| | | ttaagcaggtcaTTTACACCAACTCCTAGTAGGGGTATTATGCTAGC | |
| | | ATAATACCAGACTCTAGGGTGTTTTAGAGCTAGAAATAGCAA | |
| | | GTTAAAATAAGGCTAGTCCGTTATCAACTTGAAAAAGTGGCA | |
| | | CCGAGTCGGTGCTTTTTTTCTCGGTACCAAATTCCAGAAAAGA | |
| | | GACGCTTTTAGAGCGTCTTTTTTCGTTTTGGTCCttaaccactgtaagaa | |
| | | agttACCCAGACCGCTAAACTGAATTTACACCAACTCCTAGTAG | |
| | | GGGTATTATGCTAGCATAATACCTCCTACTAGACTGTTTTAGA | |
| | | GCTAGAAATAGCAAGTTAAAATAAGGCTAGTCCGTTATCAAC | |
| | | TTGAAAAAGTGGCACCGAGTCGGTGCTTTTTTTCTCGGTACCA | |
| | | AATTCCAGAAAAGAGACGCTGAAAAGCGTCTTTTTTTTTTTTG | |
| | | GTCCgccttgagttaggctctctcTTTACACCAACTCCTAGTAGGGGTATT | |
| | | ATGCTAGCATAATACCTCTAGAGTCCCTGTTTTAGAGCTAGA | |
| | | AATAGCAAGTTAAAATAAGGCTAGTCCGTTATCAACTTGAAA | |
| | | AAGTGGCACCGAGTCGGTGCTTTTTTTGACGAACAATAAGGC | |
| | | CTCCCTAACGGGGGGCCTTTTTTATTGATAACAAAA | |
| N12 | sgRNA | TTTACACCAACTCCTAGTAGGGGTATTATGCTAGCATAATACC | 90 |
| | array | TGTCCTAGAGGTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTAAAAAAAAAAAAGGCCTCCCAAATCGGGGGG | |
| | | CCTTTTTTATTGATAACAAAAccttgaggagctggttgtaaTTTACACCAA | |
| | | CTCCTAGTAGGGGTATTATGCTAGCATAATACCAGTGTACCT | |
| | | AGTGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCTCGGTACCAAATTCCAGAAAAGAGACGGTCGTCCGTACGA | |
| | | AGGTTTTACACCAACTCCTAGTAGGGGTATTATGCTAGCATA | |
| | | ATACCGTATGGGACTCTGTTTTAGAGCTAGAAATAGCAAGTT | |
| | | AAAATAAGGCTAGTCCGTTATCAACTTGAAAAAGTGGCACCG | |
| | | AGTCGGTGCTTTTTTTCTCGGTACCAAACCAATTATTGAAGAC | |
| | | GCTGAAAAGCGTCTTTTTTCGTTTTGGTCCtgtagaggttaagcaggtcaT | |
| | | TTACACCAACTCCTAGTAGGGGTATTATGCTAGCATAATACC | |
| | | AGACTCTAGGGTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTCTCGGTACCAAATTCCAGAAAAGAGACGCTTT | |
| | | TAGAGCGTCTTTTTTCGTTTTGGTCCttaaccactgtaagaaagttACCCA | |
| | | GACCGCTAAACTGAATTTACACCAACTCCTAGTAGGGGTATT | |
| | | ATGCTAGCATAATACCTCCTACTAGACTGTTTTAGAGCTAGA | |
| | | AATAGCAAGTTAAAATAAGGCTAGTCCGTTATCAACTTGAAA | |
| | | AAGTGGCACCGAGTCGGTGCTTTTTTTCTCGGTACCAAATTCC | |
| | | AGAAAAGAGACGCTGAAAAGCGTCTTTTTTTTTTTTGGTCCgcc | |
| | | ttgagttaggctctctcTTTACACCAACTCCTAGTAGGGGTATTATGCTA | |
| | | GCATAATACCTCTAGAGTCCCTGTTTTAGAGCTAGAAATAGC | |
| | | AAGTTAAAATAAGGCTAGTCCGTTATCAACTTGAAAAAGTGG | |
| | | CACCGAGTCGGTGCTTTTTTTGACGAACAATAAGGCCTCCCTA | |
| | | ACGGGGGGCCTTTTTTATTGATAACAAAActccgtcggagttgacgtcgT | |
| | | GCCGTTCGCTTGGGACATCTTTACACCAACTCCTAGTAGGGGT | |
| | | ATTATGCTAGCATAATACCAGGACCTAGTATGTTTTAGAGCT | |
| | | AGAAATAGCAAGTTAAAATAAGGCTAGTCCGTTATCAACTTG | |
| | | AAAAAGTGGCACCGAGTCGGTGCTTTTTTTGACGAACAATAA | |
| | | GGCCTCCCGAAAGGGGGGCCTTTTTTATTGATAACAAAActgttc | |
| | | cgcgtcacatcaacTTTACACCAACTCCTAGTAGGGGTATTATGCTAG | |
| | | CATAATACCAGTCCTACCTCTGTTTTAGAGCTAGAAATAGCA | |
| | | AGTTAAAATAAGGCTAGTCCGTTATCAACTTGAAAAAGTGGC | |
| | | ACCGAGTCGGTGCTTTTTTTTCTAACTAAAAACACCCTAACGG | |
| | | GTGTTTTTTTGTTTCTGGTCTgCCcaactcgtgatatccgcctgAGTTACCA | |
| | | AAGGTGGTCCGCTTTACACCAACTCCTAGTAGGGGTATTATG | |
| | | CTAGCATAATACCACCCCTAGGGACGTTTTAGAGCTAGAAAT | |
| | | AGCAAGTTAAAATAAGGCTAGTCCGTTATCAACTTGAAAAAG | |
| | | TGGCACCGAGTCGGTGCTTTTTTTCCAATTATTGAACACCCTT | |
| | | CGGGGTGTTTTTTTGTTTCTGGTCTCCCacgactaccgcagtgcagtaTTT | |
| | | ACACCAACTCCTAGTAGGGGTATTATGCTAGCATAATACCGA | |
| | | CTTGGACCCCGTTTTAGAGCTAGAAATAGCAAGTTAAAATAA | |
| | | GGCTAGTCCGTTATCAACTTGAAAAAGTGGCACCGAGTCGGT | |
| | | GCTTTTTTTCCAATTATTGAAGACGCTTAACAGCGTCTTTTTTT | |
| | | GTTTCTGGTCTCCCTcctgtcgttagtctccgagTCAAAGTTCGTATGGA | |
| | | AGGTTTTACACCAACTCCTAGTAGGGGTATTATGCTAGCATA | |
| | | ATACCCTCCTAGTCTAGGTTTTAGAGCTAGAAATAGCAAGTT | |
| | | AAAATAAGGCTAGTCCGTTATCAACTTGAAAAAGTGGCACCG | |
| | | AGTCGGTGCTTTTTTTTTTTCGAAAAAACACCCTAACGGGTGT | |
| | | TTTTTTGTTTCTGGTCTCCCgtgagagtactttatacgctTTTACACCAACT | |
| | | CCTAGTAGGGGTATTATGCTAGCATAATACCACTACTAGAGT | |
| | | GGTTTTAGAGCTAGAAATAGCAAGTTAAAATAAGGCTAGTCC | |
| | | GTTATCAACTTGAAAAAGTGGCACCGAGTCGGTGCTTTTTTTC | |
| | | TCGGTACCAAATCTAACTAAAAAGACGCTGAAAAGCGTCTTT | |
| | | TTTCGTTTTGGTCC | |
| N14 | sgRNA | TTTACACCAACTCCTAGTAGGGGTATTATGCTAGCATAATACC | 91 |
| | array | TGTCCTAGAGGTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTAAAAAAAAAAAAGGCCTCCCAAATCGGGGGG | |
| | | CCTTTTTTATTGATAACAAAAccttgaggagctggttgtaaTTTACACCAA | |
| | | CTCCTAGTAGGGGTATTATGCTAGCATAATACCAGTGTACCT | |
| | | AGTGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCTCGGTACCAAATTCCAGAAAAGAGACGCTTAACAGCGTCT | |
| | | TTTTTCGTTTTGGTCCtttctcagcgtaatcgttcgCGAAATCGAAGGTGA | |
| | | AGGTGTTTACACCAACTCCTAGTAGGGGTATTATGCTAGCAT | |
| | | AATACCGACATAGGATCTGTTTTAGAGCTAGAAATAGCAAGT | |
| | | TAAAATAAGGCTAGTCCGTTATCAACTTGAAAAAGTGGCACC | |
| | | GAGTCGGTGCTTTTTTTCCAATTATTGAAGGCCGCTAACGCGG | |
| | | CCTTTTTTTGTTTCTGGTCTCCCcgagattcccttatccttttTTTACACCAA | |
| | | CTCCTAGTAGGGGTATTATGCTAGCATAATACCGGGAGTCCT | |
| | | ATAGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCCAATTATTGAAGGCCTCCCAAATCGGGGGGCCTTTTTTATT | |
| | | GATAACAAAAgatcgcctcactttgaaattTATCAAAGAGTTCATGCGTTT | |
| | | TTACACCAACTCCTAGTAGGGGTATTATGCTAGCATAATACC | |
| | | CTAGGGACCCCTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTCTCGGTACCAAAAAAAAAAAAAAAGACGCTG | |
| | | AAAAGCGTCTTTTTTCGTTTTGGTCCaatagatacagttaggtttgTTTAC | |
| | | ACCAACTCCTAGTAGGGGTATTATGCTAGCATAATACCCCCT | |
| | | CTAGCTAGGTTTTAGAGCTAGAAATAGCAAGTTAAAATAAGG | |
| | | CTAGTCCGTTATCAACTTGAAAAAGTGGCACCGAGTCGGTGC | |
| | | TTTTTTTCTCGGTACCAAAAAAAAAAAAAAAGACGCTGAAAA | |
| | | GCGTCTTTTTTTTTTTTGGTCCacggagtctgagacTcggcgAAGGTCGT | |
| | | CCGTACGAAGGTTTTACACCAACTCCTAGTAGGGGTATTATG | |
| | | CTAGCATAATACCGTATGGGACTCTGTTTTAGAGCTAGAAAT | |
| | | AGCAAGTTAAAATAAGGCTAGTCCGTTATCAACTTGAAAAAG | |
| | | TGGCACCGAGTCGGTGCTTTTTTTCTCGGTACCAAACCAATTA | |
| | | TTGAAGACGCTGAAAAGCGTCTTTTTTCGTTTTGGTCCtgtagagg | |
| | | ttaagcaggtcaTTTACACCAACTCCTAGTAGGGGTATTATGCTAGC | |
| | | ATAATACCAGACTCTAGGGTGTTTTAGAGCTAGAAATAGCAA | |
| | | GTTAAAATAAGGCTAGTCCGTTATCAACTTGAAAAAGTGGCA | |
| | | CCGAGTCGGTGCTTTTTTTCTCGGTACCAAATTCCAGAAAAGA | |
| | | GACGCTTTTAGAGCGTCTTTTTTCGTTTTGGTCCttaaccactgtaagaa | |
| | | agttACCCAGACCGCTAAACTGAATTTACACCAACTCCTAGTAG | |
| | | GGGTATTATGCTAGCATAATACCTCCTACTAGACTGTTTTAGA | |
| | | GCTAGAAATAGCAAGTTAAAATAAGGCTAGTCCGTTATCAAC | |
| | | TTGAAAAAGTGGCACCGAGTCGGTGCTTTTTTTCTCGGTACCA | |
| | | AATTCCAGAAAAGAGACGCTGAAAAGCGTCTTTTTTTTTTTTG | |
| | | GTCCgccttgagttaggctctctcTTTACACCAACTCCTAGTAGGGGTATT | |
| | | ATGCTAGCATAATACCTCTAGAGTCCCTGTTTTAGAGCTAGA | |
| | | AATAGCAAGTTAAAATAAGGCTAGTCCGTTATCAACTTGAAA | |
| | | AAGTGGCACCGAGTCGGTGCTTTTTTTGACGAACAATAAGGC | |
| | | CTCCCTAACGGGGGGCCTTTTTTATTGATAACAAAActccgtcgga | |
| | | gttgacgtcgTGCCGTTCGCTTGGGACATCTTTACACCAACTCCTAG | |
| | | TAGGGGTATTATGCTAGCATAATACCAGGACCTAGTATGTTTT | |
| | | AGAGCTAGAAATAGCAAGTTAAAATAAGGCTAGTCCGTTATC | |
| | | AACTTGAAAAAGTGGCACCGAGTCGGTGCTTTTTTTGACGAA | |
| | | CAATAAGGCCTCCCGAAAGGGGGGCCTTTTTTATTGATAACA | |
| | | AAActgttccgcgtcacatcaacTTTACACCAACTCCTAGTAGGGGTATT | |
| | | ATGCTAGCATAATACCAGTCCTACCTCTGTTTTAGAGCTAGAA | |
| | | ATAGCAAGTTAAAATAAGGCTAGTCCGTTATCAACTTGAAAA | |
| | | AGTGGCACCGAGTCGGTGCTTTTTTTTCTAACTAAAAACACCC | |
| | | TAACGGGTGTTTTTTTGTTTCTGGTCTgCCcaactcgtgatatccgcctgA | |
| | | GTTACCAAAGGTGGTCCGCTTTACACCAACTCCTAGTAGGGG | |
| | | TATTATGCTAGCATAATACCACCCCTAGGGACGTTTTAGAGCT | |
| | | AGAAATAGCAAGTTAAAATAAGGCTAGTCCGTTATCAACTTG | |
| | | AAAAAGTGGCACCGAGTCGGTGCTTTTTTTCCAATTATTGAAC | |
| | | ACCCTTCGGGGTGTTTTTTTGTTTCTGGTCTCCCacgactaccgcagtg | |
| | | cagtaTTTACACCAACTCCTAGTAGGGGTATTATGCTAGCATAA | |
| | | TACCGACTTGGACCCCGTTTTAGAGCTAGAAATAGCAAGTTA | |
| | | AAATAAGGCTAGTCCGTTATCAACTTGAAAAAGTGGCACCGA | |
| | | GTCGGTGCTTTTTTTCCAATTATTGAAGACGCTTAACAGCGTC | |
| | | TTTTTTTGTTTCTGGTCTCCC | |
| N16 | sgRNA | TTTACACCAACTCCTAGTAGGGGTATTATGCTAGCATAATACC | 92 |
| | array | TGTCCTAGAGGTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTAAAAAAAAAAAAGGCCTCCCAAATCGGGGGG | |
| | | CCTTTTTTATTGATAACAAAAccttgaggagctggttgtaaTTTACACCAA | |
| | | CTCCTAGTAGGGGTATTATGCTAGCATAATACCAGTGTACCT | |
| | | AGTGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCTCGGTACCAAATTCCAGAAAAGAGACGCTTAACAGCGTCT | |
| | | TTTTTCGTTTTGGTCCtttctcagcgtaatcgttcgCGAAATCGAAGGTGA | |
| | | AGGTGTTTACACCAACTCCTAGTAGGGGTATTATGCTAGCAT | |
| | | AATACCGACATAGGATCTGTTTTAGAGCTAGAAATAGCAAGT | |
| | | TAAAATAAGGCTAGTCCGTTATCAACTTGAAAAAGTGGCACC | |
| | | GAGTCGGTGCTTTTTTTCCAATTATTGAAGGCCGCTAACGCGG | |
| | | CCTTTTTTTGTTTCTGGTCTCCCcgagattcccttatccttttTTTACACCAA | |
| | | CTCCTAGTAGGGGTATTATGCTAGCATAATACCGGGAGTCCT | |
| | | ATAGTTTTAGAGCTAGAAATAGCAAGTTAAAATAAGGCTAGT | |
| | | CCGTTATCAACTTGAAAAAGTGGCACCGAGTCGGTGCTTTTTT | |
| | | TCCAATTATTGAAGGCCTCCCAAATCGGGGGGCCTTTTTTATT | |
| | | GATAACAAAAgatcgcctcactttgaaattTATCAAAGAGTTCATGCGTTT | |
| | | TTACACCAACTCCTAGTAGGGGTATTATGCTAGCATAATACC | |
| | | CTAGGGACCCCTGTTTTAGAGCTAGAAATAGCAAGTTAAAAT | |
| | | AAGGCTAGTCCGTTATCAACTTGAAAAAGTGGCACCGAGTCG | |
| | | GTGCTTTTTTTCTCGGTACCAAAAAAAAAAAAAAAGACGCTG | |
| | | AAAAGCGTCTTTTTTCGTTTTGGTCCaatagatacagttaggtttgTTTAC | |
| | | ACCAACTCCTAGTAGGGGTATTATGCTAGCATAATACCCCCT | |
| | | CTAGCTAGGTTTTAGAGCTAGAAATAGCAAGTTAAAATAAGG | |
| | | CTAGTCCGTTATCAACTTGAAAAAGTGGCACCGAGTCGGTGC | |
| | | TTTTTTTCTCGGTACCAAAAAAAAAAAAAAAGACGCTGAAAA | |
| | | GCGTCTTTTTTTTTTTTGGTCCacggagtctgagacTcggcgAAGGTCGT | |
| | | CCGTACGAAGGTTTTACACCAACTCCTAGTAGGGGTATTATG | |
| | | CTAGCATAATACCGTATGGGACTCTGTTTTAGAGCTAGAAAT | |
| | | AGCAAGTTAAAATAAGGCTAGTCCGTTATCAACTTGAAAAAG | |
| | | TGGCACCGAGTCGGTGCTTTTTTTCTCGGTACCAAACCAATTA | |
| | | TTGAAGACGCTGAAAAGCGTCTTTTTTCGTTTTGGTCCtgtagagg | |
| | | ttaagcaggtcaTTTACACCAACTCCTAGTAGGGGTATTATGCTAGC | |
| | | ATAATACCAGACTCTAGGGTGTTTTAGAGCTAGAAATAGCAA | |
| | | GTTAAAATAAGGCTAGTCCGTTATCAACTTGAAAAAGTGGCA | |
| | | CCGAGTCGGTGCTTTTTTTCTCGGTACCAAATTCCAGAAAAGA | |
| | | GACGCTTTTAGAGCGTCTTTTTTCGTTTTGGTCCttaaccactgtaagaa | |
| | | agttACCCAGACCGCTAAACTGAATTTACACCAACTCCTAGTAG | |
| | | GGGTATTATGCTAGCATAATACCTCCTACTAGACTGTTTTAGA | |
| | | GCTAGAAATAGCAAGTTAAAATAAGGCTAGTCCGTTATCAAC | |
| | | TTGAAAAAGTGGCACCGAGTCGGTGCTTTTTTTCTCGGTACCA | |
| | | AATTCCAGAAAAGAGACGCTGAAAAGCGTCTTTTTTTTTTTTG | |
| | | GTCCgccttgagttaggctctctcTTTACACCAACTCCTAGTAGGGGTATT | |
| | | ATGCTAGCATAATACCTCTAGAGTCCCTGTTTTAGAGCTAGA | |
| | | AATAGCAAGTTAAAATAAGGCTAGTCCGTTATCAACTTGAAA | |
| | | AAGTGGCACCGAGTCGGTGCTTTTTTTGACGAACAATAAGGC | |
| | | CTCCCTAACGGGGGGCCTTTTTTATTGATAACAAAActccgtcgga | |
| | | gttgacgtcgTGCCGTTCGCTTGGGACATCTTTACACCAACTCCTAG | |
| | | TAGGGGTATTATGCTAGCATAATACCAGGACCTAGTATGTTTT | |
| | | AGAGCTAGAAATAGCAAGTTAAAATAAGGCTAGTCCGTTATC | |
| | | AACTTGAAAAAGTGGCACCGAGTCGGTGCTTTTTTTGACGAA | |
| | | CAATAAGGCCTCCCGAAAGGGGGGCCTTTTTTATTGATAACA | |
| | | AAActgttccgcgtcacatcaacTTTACACCAACTCCTAGTAGGGGTATT | |
| | | ATGCTAGCATAATACCAGTCCTACCTCTGTTTTAGAGCTAGAA | |
| | | ATAGCAAGTTAAAATAAGGCTAGTCCGTTATCAACTTGAAAA | |
| | | AGTGGCACCGAGTCGGTGCTTTTTTTTCTAACTAAAAACACCC | |
| | | TAACGGGTGTTTTTTTGTTTCTGGTCTgCCcaactcgtgatatccgcctgA | |
| | | GTTACCAAAGGTGGTCCGCTTTACACCAACTCCTAGTAGGGG | |
| | | TATTATGCTAGCATAATACCACCCCTAGGGACGTTTTAGAGCT | |
| | | AGAAATAGCAAGTTAAAATAAGGCTAGTCCGTTATCAACTTG | |
| | | AAAAAGTGGCACCGAGTCGGTGCTTTTTTTCCAATTATTGAAC | |
| | | ACCCTTCGGGGTGTTTTTTTGTTTCTGGTCTCCCacgactaccgcagtg | |
| | | cagtaTTTACACCAACTCCTAGTAGGGGTATTATGCTAGCATAA | |
| | | TACCGACTTGGACCCCGTTTTAGAGCTAGAAATAGCAAGTTA | |
| | | AAATAAGGCTAGTCCGTTATCAACTTGAAAAAGTGGCACCGA | |
| | | GTCGGTGCTTTTTTTCCAATTATTGAAGACGCTTAACAGCGTC | |
| | | TTTTTTTGTTTCTGGTCTCCCTcctgtcgttagtctccgagTCAAAGTTCG | |
| | | TATGGAAGGTTTTACACCAACTCCTAGTAGGGGTATTATGCT | |
| | | AGCATAATACCCTCCTAGTCTAGGTTTTAGAGCTAGAAATAG | |
| | | CAAGTTAAAATAAGGCTAGTCCGTTATCAACTTGAAAAAGTG | |
| | | GCACCGAGTCGGTGCTTTTTTTTTTTCGAAAAAACACCCTAAC | |
| | | GGGTGTTTTTTTGTTTCTGGTCTCCCgtgagagtactttatacgctTTTACA | |
| | | CCAACTCCTAGTAGGGGTATTATGCTAGCATAATACCACTAC | |
| | | TAGAGTGGTTTTAGAGCTAGAAATAGCAAGTTAAAATAAGGC | |
| | | TAGTCCGTTATCAACTTGAAAAAGTGGCACCGAGTCGGTGCT | |
| | | TTTTTTCTCGGTACCAAATCTAACTAAAAAGACGCTGAAAAG | |
| | | CGTCTTTTTTCGTTTTGGTCC | |
| RiboJ | insulator | AGCTGTCACCGGATGTGCTTTCCGGTCTGATGAGTCCGTGAG | 93 |
| | | GACGAAACAGCCTCTACAAATAATTTTGTTTAA | |
| dCas9 | gene | ATGGATAAGAAATACTCAATAGGCTTAGCTATCGGCACAAAT | 94 |
| | | AGCGTCGGATGGGCGGTGATCACTGATGAATATAAGGTTCCG | |
| | | TCTAAAAAGTTCAAGGTTCTGGGAAATACAGACCGCCACAGT | |
| | | ATCAAAAAAAATCTTATAGGGGCTCTTTTATTTGACAGTGGA | |
| | | GAGACAGCGGAAGCGACTCGTCTCAAACGGACAGCTCGTAG | |
| | | AAGGTATACACGTCGGAAGAATCGTATTTGTTATCTACAGGA | |
| | | GATTTTTTCAAATGAGATGGCGAAAGTAGATGATAGTTTCTTT | |
| | | CATCGACTTGAAGAGTCTTTTTTGGTGGAAGAAGACAAGAAG | |
| | | CATGAACGTCATCCTATTTTTGGAAATATAGTAGATGAAGTT | |
| | | GCTTATCATGAGAAATATCCAACTATCTATCATCTGCGAAAA | |
| | | AAATTGGTAGATTCTACTGATAAAGCGGATTTGCGCTTAATCT | |
| | | ATTTGGCCTTAGCGCATATGATTAAGTTTCGTGGTCATTTTTT | |
| | | GATTGAGGGAGATTTAAATCCTGATAATAGTGATGTGGACAA | |
| | | ACTATTTATCCAGTTGGTACAAACCTACAATCAATTATTTGAA | |
| | | GAAAACCCTATTAACGCAAGTGGAGTAGATGCTAAAGCGATT | |
| | | CTTTCTGCACGATTGAGTAAATCAAGACGATTAGAAAATCTC | |
| | | ATTGCTCAGCTCCCCGGTGAGAAGAAAAATGGCTTATTTGGG | |
| | | AATCTCATTGCTTTGTCATTGGGTTTGACCCCTAATTTTAAAT | |
| | | CAAATTTTGATTTGGCAGAAGATGCTAAATTACAGCTTTCAA | |
| | | AAGATACTTACGATGATGATTTAGATAATTTATTGGCGCAAA | |
| | | TTGGAGATCAATATGCTGATTTGTTTTTGGCAGCTAAGAATTT | |
| | | ATCAGATGCTATTTTACTTTCAGATATCCTAAGAGTAAATACT | |
| | | GAAATAACTAAGGCTCCCCTATCAGCTTCAATGATTAAACGC | |
| | | TACGATGAACATCATCAAGACTTGACTCTTTTAAAAGCTTTAG | |
| | | TTCGACAACAACTTCCAGAAAAGTATAAAGAAATCTTTTTTG | |
| | | ATCAATCAAAAAACGGATATGCAGGTTATATTGATGGGGGAG | |
| | | CTAGCCAAGAAGAATTTTATAAATTTATCAAACCAATTTTAG | |
| | | AAAAAATGGATGGTACTGAGGAATTATTGGTGAAACTAAATC | |
| | | GTGAAGATTTGCTGCGCAAGCAACGGACCTTTGACAACGGCT | |
| | | CTATTCCCCATCAAATTCACTTGGGTGAGCTGCATGCTATTTT | |
| | | GAGAAGACAAGAAGACTTTTATCCATTTTTAAAAGACAATCG | |
| | | TGAGAAGATTGAAAAAATCTTGACTTTTCGAATTCCTTATTAT | |
| | | GTTGGTCCATTGGCGCGTGGCAATAGTCGTTTTGCATGGATG | |
| | | ACTCGGAAGTCTGAAGAAACAATTACCCCATGGAATTTTGAA | |
| | | GAAGTTGTCGATAAAGGTGCTTCAGCTCAATCATTTATTGAA | |
| | | CGCATGACAAACTTTGATAAAAATCTTCCAAATGAAAAAGTA | |
| | | CTACCAAAACATAGTTTGCTTTATGAGTATTTTACGGTTTATA | |
| | | ACGAATTGACAAAGGTCAAATATGTTACTGAAGGAATGCGAA | |
| | | AACCAGCATTTCTTTCAGGTGAACAGAAGAAAGCCATTGTTG | |
| | | ATTTACTCTTCAAAACAAATCGAAAAGTAACCGTTAAGCAAT | |
| | | TAAAAGAAGATTATTTCAAAAAAATAGAATGTTTTGATAGTG | |
| | | TTGAAATTTCAGGAGTTGAAGATAGATTTAATGCTTCATTAG | |
| | | GTACCTACCATGATTTGCTAAAAATTATTAAAGATAAAGATT | |
| | | TTTTGGATAATGAAGAAAATGAAGATATCTTAGAGGATATTG | |
| | | TTTTAACATTGACCTTATTTGAAGATAGGGAGATGATTGAGG | |
| | | AAAGACTTAAAACATATGCTCACCTCTTTGATGATAAGGTGA | |
| | | TGAAACAGCTTAAACGTCGCCGTTATACTGGTTGGGGACGTT | |
| | | TGTCTCGAAAATTGATTAATGGTATTAGGGATAAGCAATCTG | |
| | | GCAAAACAATATTAGATTTTTTGAAATCAGATGGTTTTGCCA | |
| | | ATCGCAATTTTATGCAGCTGATCCATGATGATAGTTTGACATT | |
| | | TAAAGAAGACATTCAAAAAGCACAAGTGTCTGGACAAGGCG | |
| | | ATAGTTTACATGAACATATTGCAAATTTAGCTGGTAGCCCTGC | |
| | | TATTAAAAAAGGTATTTTACAGACTGTAAAAGTTGTTGATGA | |
| | | ATTGGTCAAAGTAATGGGGCGGCATAAGCCAGAAAATATCGT | |
| | | TATTGAAATGGCACGTGAAAATCAGACAACTCAAAAGGGCC | |
| | | AGAAAAATTCGCGAGAGCGTATGAAACGAATCGAAGAAGGT | |
| | | ATCAAAGAATTAGGAAGTCAGATTCTTAAAGAGCATCCTGTT | |
| | | GAAAATACTCAATTGCAAAATGAAAAGCTCTATCTCTATTAT | |
| | | CTCCAAAATGGAAGAGACATGTATGTGGACCAAGAATTAGAT | |
| | | ATTAATCGTTTAAGTGATTATGATGTCGATGCCATTGTTCCAC | |
| | | AAAGTTTCCTTAAAGACGATTCAATAGACAATAAGGTCTTAA | |
| | | CGCGTTCTGATAAAAATCGTGGTAAATCGGATAACGTTCCAA | |
| | | GTGAAGAAGTAGTCAAAAAGATGAAAAACTATTGGAGACAA | |
| | | CTTCTAAACGCCAAGTTAATCACTCAACGTAAGTTTGATAATT | |
| | | TAACGAAAGCTGAACGTGGAGGTTTGAGTGAACTTGATAAAG | |
| | | CTGGTTTTATCAAACGCCAATTGGTTGAAACTCGCCAAATCA | |
| | | CTAAGCATGTGGCACAAATTTTGGATAGTCGCATGAATACTA | |
| | | AATACGATGAAAATGATAAACTTATTCGAGAGGTTAAAGTGA | |
| | | TTACCTTAAAATCTAAATTAGTTTCTGACTTCCGAAAAGATTT | |
| | | CCAATTCTATAAAGTACGTGAGATTAACAATTACCATCATGC | |
| | | CCATGATGCGTATCTAAATGCCGTCGTTGGAACTGCTTTGATT | |
| | | AAGAAATATCCAAAACTTGAATCGGAGTTTGTCTATGGTGAT | |
| | | TATAAAGTTTATGATGTTCGTAAAATGATTGCTAAGTCTGAGC | |
| | | AAGAAATAGGCAAAGCAACCGCAAAATATTTCTTTTACTCTA | |
| | | ATATCATGAACTTCTTCAAAACAGAAATTACACTTGCAAATG | |
| | | GAGAGATTCGCAAACGCCCTCTAATCGAAACTAATGGGGAAA | |
| | | CTGGAGAAATTGTCTGGGATAAAGGGCGAGATTTTGCCACAG | |
| | | TGCGCAAAGTATTGTCCATGCCCCAAGTCAATATTGTCAAGA | |
| | | AAACAGAAGTACAGACAGGCGGATTCTCCAAGGAGTCAATTT | |
| | | TACCAAAAAGAAATTCGGACAAGCTTATTGCTCGTAAAAAAG | |
| | | ACTGGGATCCAAAAAAATATGGTGGTTTTGATAGTCCAACGG | |
| | | TAGCTTATTCAGTCCTAGTGGTTGCTAAGGTGGAAAAAGGGA | |
| | | AATCGAAGAAGTTAAAATCCGTTAAAGAGTTACTAGGGATCA | |
| | | CAATTATGGAAAGAAGTTCCTTTGAAAAAAATCCGATTGACT | |
| | | TTTTAGAAGCTAAAGGATATAAGGAAGTTAAAAAAGACTTAA | |
| | | TCATTAAACTACCTAAATATAGTCTTTTTGAGTTAGAAAACGG | |
| | | TCGTAAACGGATGCTGGCTAGTGCCGGAGAATTACAAAAAGG | |
| | | AAATGAGCTGGCTCTGCCAAGCAAATATGTGAATTTTTTATAT | |
| | | TTAGCTAGTCATTATGAAAAGTTGAAGGGTAGTCCAGAAGAT | |
| | | AACGAACAAAAACAATTGTTTGTGGAGCAGCATAAGCATTAT | |
| | | TTAGATGAGATTATTGAGCAAATCAGTGAATTTTCTAAGCGT | |
| | | GTTATTTTAGCAGATGCCAATTTAGATAAAGTTCTTAGTGCAT | |
| | | ATAACAAACATAGAGACAAACCAATACGTGAACAAGCAGAA | |
| | | AATATTATTCATTTATTTACGTTGACGAATCTTGGAGCTCCCG | |
| | | CTGCTTTTAAATATTTTGATACAACAATTGATCGTAAACGATA | |
| | | TACGTCTACAAAAGAAGTTTTAGATGCCACTCTTATCCATCAA | |
| | | TCCATCACTGGTCTTTATGAAACACGCATTGATTTGAGTCAGC | |
| | | TAGGAGGTGACTAA | |
| dCas9*_ZFP | gene | ATGGATAAGAAATACTCAATAGGCTTAGCTATCGGCACAAAT | 95 |
| | | AGCGTCGGATGGGCGGTGATCACTGATGAATATAAGGTTCCG | |
| | | TCTAAAAAGTTCAAGGTTCTGGGAAATACAGACCGCCACAGT | |
| | | ATCAAAAAAAATCTTATAGGGGCTCTTTTATTTGACAGTGGA | |
| | | GAGACAGCGGAAGCGACTCGTCTCAAACGGACAGCTCGTAG | |
| | | AAGGTATACACGTCGGAAGAATCGTATTTGTTATCTACAGGA | |
| | | GATTTTTTCAAATGAGATGGCGAAAGTAGATGATAGTTTCTTT | |
| | | CATCGACTTGAAGAGTCTTTTTTGGTGGAAGAAGACAAGAAG | |
| | | CATGAACGTCATCCTATTTTTGGAAATATAGTAGATGAAGTT | |
| | | GCTTATCATGAGAAATATCCAACTATCTATCATCTGCGAAAA | |
| | | AAATTGGTAGATTCTACTGATAAAGCGGATTTGCGCTTAATCT | |
| | | ATTTGGCCTTAGCGCATATGATTAAGTTTCGTGGTCATTTTTT | |
| | | GATTGAGGGAGATTTAAATCCTGATAATAGTGATGTGGACAA | |
| | | ACTATTTATCCAGTTGGTACAAACCTACAATCAATTATTTGAA | |
| | | GAAAACCCTATTAACGCAAGTGGAGTAGATGCTAAAGCGATT | |
| | | CTTTCTGCACGATTGAGTAAATCAAGACGATTAGAAAATCTC | |
| | | ATTGCTCAGCTCCCCGGTGAGAAGAAAAATGGCTTATTTGGG | |
| | | AATCTCATTGCTTTGTCATTGGGTTTGACCCCTAATTTTAAAT | |
| | | CAAATTTTGATTTGGCAGAAGATGCTAAATTACAGCTTTCAA | |
| | | AAGATACTTACGATGATGATTTAGATAATTTATTGGCGCAAA | |
| | | TTGGAGATCAATATGCTGATTTGTTTTTGGCAGCTAAGAATTT | |
| | | ATCAGATGCTATTTTACTTTCAGATATCCTAAGAGTAAATACT | |
| | | GAAATAACTAAGGCTCCCCTATCAGCTTCAATGATTAAACGC | |
| | | TACGATGAACATCATCAAGACTTGACTCTTTTAAAAGCTTTAG | |
| | | TTCGACAACAACTTCCAGAAAAGTATAAAGAAATCTTTTTTG | |
| | | ATCAATCAAAAAACGGATATGCAGGTTATATTGATGGGGGAG | |
| | | CTAGCCAAGAAGAATTTTATAAATTTATCAAACCAATTTTAG | |
| | | AAAAAATGGATGGTACTGAGGAATTATTGGTGAAACTAAATC | |
| | | GTGAAGATTTGCTGCGCAAGCAACGGACCTTTGACAACGGCT | |
| | | CTATTCCCCATCAAATTCACTTGGGTGAGCTGCATGCTATTTT | |
| | | GAGAAGACAAGAAGACTTTTATCCATTTTTAAAAGACAATCG | |
| | | TGAGAAGATTGAAAAAATCTTGACTTTTCGAATTCCTTATTAT | |
| | | GTTGGTCCATTGGCGCGTGGCAATAGTCGTTTTGCATGGATG | |
| | | ACTCGGAAGTCTGAAGAAACAATTACCCCATGGAATTTTGAA | |
| | | GAAGTTGTCGATAAAGGTGCTTCAGCTCAATCATTTATTGAA | |
| | | CGCATGACAAACTTTGATAAAAATCTTCCAAATGAAAAAGTA | |
| | | CTACCAAAACATAGTTTGCTTTATGAGTATTTTACGGTTTATA | |
| | | ACGAATTGACAAAGGTCAAATATGTTACTGAAGGAATGCGAA | |
| | | AACCAGCATTTCTTTCAGGTGAACAGAAGAAAGCCATTGTTG | |
| | | ATTTACTCTTCAAAACAAATCGAAAAGTAACCGTTAAGCAAT | |
| | | TAAAAGAAGATTATTTCAAAAAAATAGAATGTTTTGATAGTG | |
| | | TTGAAATTTCAGGAGTTGAAGATAGATTTAATGCTTCATTAG | |
| | | GTACCTACCATGATTTGCTAAAAATTATTAAAGATAAAGATT | |
| | | TTTTGGATAATGAAGAAAATGAAGATATCTTAGAGGATATTG | |
| | | TTTTAACATTGACCTTATTTGAAGATAGGGAGATGATTGAGG | |
| | | AAAGACTTAAAACATATGCTCACCTCTTTGATGATAAGGTGA | |
| | | TGAAACAGCTTAAACGTCGCCGTTATACTGGTTGGGGACGTT | |
| | | TGTCTCGAAAATTGATTAATGGTATTAGGGATAAGCAATCTG | |
| | | GCAAAACAATATTAGATTTTTTGAAATCAGATGGTTTTGCCA | |
| | | ATCGCAATTTTATGCAGCTGATCCATGATGATAGTTTGACATT | |
| | | TAAAGAAGACATTCAAAAAGCACAAGTGTCTGGACAAGGCG | |
| | | ATAGTTTACATGAACATATTGCAAATTTAGCTGGTAGCCCTGC | |
| | | TATTAAAAAAGGTATTTTACAGACTGTAAAAGTTGTTGATGA | |
| | | ATTGGTCAAAGTAATGGGGCGGCATAAGCCAGAAAATATCGT | |
| | | TATTGAAATGGCACGTGAAAATCAGACAACTCAAAAGGGCC | |
| | | AGAAAAATTCGCGAGAGCGTATGAAACGAATCGAAGAAGGT | |
| | | ATCAAAGAATTAGGAAGTCAGATTCTTAAAGAGCATCCTGTT | |
| | | GAAAATACTCAATTGCAAAATGAAAAGCTCTATCTCTATTAT | |
| | | CTCCAAAATGGAAGAGACATGTATGTGGACCAAGAATTAGAT | |
| | | ATTAATCGTTTAAGTGATTATGATGTCGATGCCATTGTTCCAC | |
| | | AAAGTTTCCTTAAAGACGATTCAATAGACAATAAGGTCTTAA | |
| | | CGCGTTCTGATAAAAATCGTGGTAAATCGGATAACGTTCCAA | |
| | | GTGAAGAAGTAGTCAAAAAGATGAAAAACTATTGGAGACAA | |
| | | CTTCTAAACGCCAAGTTAATCACTCAACGTAAGTTTGATAATT | |
| | | TAACGAAAGCTGAACGTGGAGGTTTGAGTGAACTTGATAAAG | |
| | | CTGGTTTTATCAAACGCCAATTGGTTGAAACTCGCCAAATCA | |
| | | CTAAGCATGTGGCACAAATTTTGGATAGTCGCATGAATACTA | |
| | | AATACGATGAAAATGATAAACTTATTCGAGAGGTTAAAGTGA | |
| | | TTACCTTAAAATCTAAATTAGTTTCTGACTTCCGAAAAGATTT | |
| | | CCAATTCTATAAAGTACGTGAGATTAACAATTACCATCATGC | |
| | | CCATGATGCGTATCTAAATGCCGTCGTTGGAACTGCTTTGATT | |
| | | AAGAAATATCCAAAACTTGAATCGGAGTTTGTCTATGGTGAT | |
| | | TATAAAGTTTATGATGTTCGTAAAATGATTGCTAAGTCTGAGC | |
| | | AAGAAATAGGCAAAGCAACCGCAAAATATTTCTTTTACTCTA | |
| | | ATATCATGAACTTCTTCAAAACAGAAATTACACTTGCAAATG | |
| | | GAGAGATTCGCAAACGCCCTCTAATCGAAACTAATGGGGAAA | |
| | | CTGGAGAAATTGTCTGGGATAAAGGGCGAGATTTTGCCACAG | |
| | | TGCGCAAAGTATTGTCCATGCCCCAAGTCAATATTGTCAAGA | |
| | | AAACAGAAGTACAGACAGGCGGATTCTCCAAGGAGTCAATTT | |
| | | TACCAAAAAGAAATTCGGACAAGCTTATTGCTCGTAAAAAAG | |
| | | ACTGGGATCCAAAAAAATATGGTGGTTTTGATAGTCCAACGG | |
| | | TAGCTTATTCAGTCCTAGTGGTTGCTAAGGTGGAAAAAGGGA | |
| | | AATCGAAGAAGTTAAAATCCGTTAAAGAGTTACTAGGGATCA | |
| | | CAATTATGGAAAGAAGTTCCTTTGAAAAAAATCCGATTGACT | |
| | | TTTTAGAAGCTAAAGGATATAAGGAAGTTAAAAAAGACTTAA | |
| | | TCATTAAACTACCTAAATATAGTCTTTTTGAGTTAGAAAACGG | |
| | | TCGTAAACGGATGCTGGCTAGTGCCGGAGAATTACAAAAAGG | |
| | | AAATGAGCTGGCTCTGCCAAGCAAATATGTGAATTTTTTATAT | |
| | | TTAGCTAGTCATTATGAAAAGTTGAAGGGTAGTCCAGAAGAT | |
| | | AACGAACAAAAACAATTGTTTGTGGAGCAGCATAAGCATTAT | |
| | | TTAGATGAGATTATTGAGCAAATCAGTGAATTTTCTAAGCGT | |
| | | GTTATTTTAGCAGATGCCAATTTAGATAAAGTTCTTAGTGCAT | |
| | | ATAACAAACATAGAGACAAACCAATACGTGAACAAGCAGAA | |
| | | AATATTATTCATTTATTTACGTTGACGAATCTTGGAGCTCCCG | |
| | | CTGCTTTTAAATATTTTGATACAACAATTGATCGTAAAAAGTA | |
| | | TACGTCTACAAAAGAAGTTTTAGATGCCACTCTTATCCATCAA | |
| | | TCCATCACTGGTCTTTATGAAACACGCATTGATTTGAGTCAGC | |
| | | TAGGAGGTGACGGCACCGGCGGGCCCAAGAAGAAGAGGAAG | |
| | | GTATACCCATACGATGTTCCTGACTATGCGGGCTATCCCTATG | |
| | | ACGTCCCGGACTATGCAGGATCGTATCCTTATGACGTTCCAG | |
| | | ATTACGCTGGATCCGCCGCTCCGGCAGCTAAGAAAAAGAAAC | |
| | | TGGATTTCGAATCCGGAAAGCCCTATAAATGTCCTGAATGTG | |
| | | GCAAGTCCTTCTCGCGGAGCGACGACCTGACACGGCACCAAC | |
| | | GTACGCACACTGGTGAGAAGCCATACGCGTGTCCTGTCGAGT | |
| | | CCTGTGACCGCCGCTTCAGTCAGAAGGGACACCTGACACGGC | |
| | | ACATCCGCATTCACACAGGGCAAAAACCGTTTCAATGCCGCA | |
| | | TCTGCATGAGGAACTTCAGCATCCGTAGCAGCCTGACACGGC | |
| | | ACATCCGCACCCACACAGGAGAAAAGCCCTTCGCCTGTGACA | |
| | | TCTGCGGCAGGAAGTTCGCGCTGAGCCACCACCTGACACGGC | |
| | | ACACCAAGATCCACCTCCGTCAGAAAGACCCCGGGTAA | |
| dCas9*_HNH | gene | ATGGATAAGAAATACTCAATAGGCTTAGCTATCGGCACAAAT | 96 |
| | | AGCGTCGGATGGGCGGTGATCACTGATGAATATAAGGTTCCG | |
| | | TCTAAAAAGTTCAAGGTTCTGGGAAATACAGACCGCCACAGT | |
| | | ATCAAAAAAAATCTTATAGGGGCTCTTTTATTTGACAGTGGA | |
| | | GAGACAGCGGAAGCGACTCGTCTCAAACGGACAGCTCGTAG | |
| | | AAGGTATACACGTCGGAAGAATCGTATTTGTTATCTACAGGA | |
| | | GATTTTTTCAAATGAGATGGCGAAAGTAGATGATAGTTTCTTT | |
| | | CATCGACTTGAAGAGTCTTTTTTGGTGGAAGAAGACAAGAAG | |
| | | CATGAACGTCATCCTATTTTTGGAAATATAGTAGATGAAGTT | |
| | | GCTTATCATGAGAAATATCCAACTATCTATCATCTGCGAAAA | |
| | | AAATTGGTAGATTCTACTGATAAAGCGGATTTGCGCTTAATCT | |
| | | ATTTGGCCTTAGCGCATATGATTAAGTTTCGTGGTCATTTTTT | |
| | | GATTGAGGGAGATTTAAATCCTGATAATAGTGATGTGGACAA | |
| | | ACTATTTATCCAGTTGGTACAAACCTACAATCAATTATTTGAA | |
| | | GAAAACCCTATTAACGCAAGTGGAGTAGATGCTAAAGCGATT | |
| | | CTTTCTGCACGATTGAGTAAATCAAGACGATTAGAAAATCTC | |
| | | ATTGCTCAGCTCCCCGGTGAGAAGAAAAATGGCTTATTTGGG | |
| | | AATCTCATTGCTTTGTCATTGGGTTTGACCCCTAATTTTAAAT | |
| | | CAAATTTTGATTTGGCAGAAGATGCTAAATTACAGCTTTCAA | |
| | | AAGATACTTACGATGATGATTTAGATAATTTATTGGCGCAAA | |
| | | TTGGAGATCAATATGCTGATTTGTTTTTGGCAGCTAAGAATTT | |
| | | ATCAGATGCTATTTTACTTTCAGATATCCTAAGAGTAAATACT | |
| | | GAAATAACTAAGGCTCCCCTATCAGCTTCAATGATTAAACGC | |
| | | TACGATGAACATCATCAAGACTTGACTCTTTTAAAAGCTTTAG | |
| | | TTCGACAACAACTTCCAGAAAAGTATAAAGAAATCTTTTTTG | |
| | | ATCAATCAAAAAACGGATATGCAGGTTATATTGATGGGGGAG | |
| | | CTAGCCAAGAAGAATTTTATAAATTTATCAAACCAATTTTAG | |
| | | AAAAAATGGATGGTACTGAGGAATTATTGGTGAAACTAAATC | |
| | | GTGAAGATTTGCTGCGCAAGCAACGGACCTTTGACAACGGCT | |
| | | CTATTCCCCATCAAATTCACTTGGGTGAGCTGCATGCTATTTT | |
| | | GAGAAGACAAGAAGACTTTTATCCATTTTTAAAAGACAATCG | |
| | | TGAGAAGATTGAAAAAATCTTGACTTTTCGAATTCCTTATTAT | |
| | | GTTGGTCCATTGGCGCGTGGCAATAGTCGTTTTGCATGGATG | |
| | | ACTCGGAAGTCTGAAGAAACAATTACCCCATGGAATTTTGAA | |
| | | GAAGTTGTCGATAAAGGTGCTTCAGCTCAATCATTTATTGAA | |
| | | CGCATGACAAACTTTGATAAAAATCTTCCAAATGAAAAAGTA | |
| | | CTACCAAAACATAGTTTGCTTTATGAGTATTTTACGGTTTATA | |
| | | ACGAATTGACAAAGGTCAAATATGTTACTGAAGGAATGCGAA | |
| | | AACCAGCATTTCTTTCAGGTGAACAGAAGAAAGCCATTGTTG | |
| | | ATTTACTCTTCAAAACAAATCGAAAAGTAACCGTTAAGCAAT | |
| | | TAAAAGAAGATTATTTCAAAAAAATAGAATGTTTTGATAGTG | |
| | | TTGAAATTTCAGGAGTTGAAGATAGATTTAATGCTTCATTAG | |
| | | GTACCTACCATGATTTGCTAAAAATTATTAAAGATAAAGATT | |
| | | TTTTGGATAATGAAGAAAATGAAGATATCTTAGAGGATATTG | |
| | | TTTTAACATTGACCTTATTTGAAGATAGGGAGATGATTGAGG | |
| | | AAAGACTTAAAACATATGCTCACCTCTTTGATGATAAGGTGA | |
| | | TGAAACAGCTTAAACGTCGCCGTTATACTGGTTGGGGACGTT | |
| | | TGTCTCGAAAATTGATTAATGGTATTAGGGATAAGCAATCTG | |
| | | GCAAAACAATATTAGATTTTTTGAAATCAGATGGTTTTGCCA | |
| | | ATCGCAATTTTATGCAGCTGATCCATGATGATAGTTTGACATT | |
| | | TAAAGAAGACATTCAAAAAGCACAAGTGTCTGGACAAGGCG | |
| | | ATAGTTTACATGAACATATTGCAAATTTAGCTGGTAGCCCTGC | |
| | | TATTAAAAAAGGTATTTTACAGACTGTAAAAGTTGTTGATGA | |
| | | ATTGGTCAAAGTAATGGGGCGGCATAAGCCAGAAAATATCGT | |
| | | TATTGAAATGGCACGTGAAAATCAGGGAGGTTCAGGTGGATC | |
| | | GCGCCAATTGGTTGAAACTCGCCAAATCACTAAGCATGTGGC | |
| | | ACAAATTTTGGATAGTCGCATGAATACTAAATACGATGAAAA | |
| | | TGATAAACTTATTCGAGAGGTTAAAGTGATTACCTTAAAATC | |
| | | TAAATTAGTTTCTGACTTCCGAAAAGATTTCCAATTCTATAAA | |
| | | GTACGTGAGATTAACAATTACCATCATGCCCATGATGCGTAT | |
| | | CTAAATGCCGTCGTTGGAACTGCTTTGATTAAGAAATATCCA | |
| | | AAACTTGAATCGGAGTTTGTCTATGGTGATTATAAAGTTTATG | |
| | | ATGTTCGTAAAATGATTGCTAAGTCTGAGCAAGAAATAGGCA | |
| | | AAGCAACCGCAAAATATTTCTTTTACTCTAATATCATGAACTT | |
| | | CTTCAAAACAGAAATTACACTTGCAAATGGAGAGATTCGCAA | |
| | | ACGCCCTCTAATCGAAACTAATGGGGAAACTGGAGAAATTGT | |
| | | CTGGGATAAAGGGCGAGATTTTGCCACAGTGCGCAAAGTATT | |
| | | GTCCATGCCCCAAGTCAATATTGTCAAGAAAACAGAAGTACA | |
| | | GACAGGCGGATTCTCCAAGGAGTCAATTTTACCAAAAAGAAA | |
| | | TTCGGACAAGCTTATTGCTCGTAAAAAAGACTGGGATCCAAA | |
| | | AAAATATGGTGGTTTTGATAGTCCAACGGTAGCTTATTCAGTC | |
| | | CTAGTGGTTGCTAAGGTGGAAAAAGGGAAATCGAAGAAGTT | |
| | | AAAATCCGTTAAAGAGTTACTAGGGATCACAATTATGGAAAG | |
| | | AAGTTCCTTTGAAAAAAATCCGATTGACTTTTTAGAAGCTAA | |
| | | AGGATATAAGGAAGTTAAAAAAGACTTAATCATTAAACTACC | |
| | | TAAATATAGTCTTTTTGAGTTAGAAAACGGTCGTAAACGGAT | |
| | | GCTGGCTAGTGCCGGAGAATTACAAAAAGGAAATGAGCTGG | |
| | | CTCTGCCAAGCAAATATGTGAATTTTTTATATTTAGCTAGTCA | |
| | | TTATGAAAAGTTGAAGGGTAGTCCAGAAGATAACGAACAAA | |
| | | AACAATTGTTTGTGGAGCAGCATAAGCATTATTTAGATGAGA | |
| | | TTATTGAGCAAATCAGTGAATTTTCTAAGCGTGTTATTTTAGC | |
| | | AGATGCCAATTTAGATAAAGTTCTTAGTGCATATAACAAACA | |
| | | TAGAGACAAACCAATACGTGAACAAGCAGAAAATATTATTCA | |
| | | TTTATTTACGTTGACGAATCTTGGAGCTCCCGCTGCTTTTAAA | |
| | | TATTTTGATACAACAATTGATCGTAAAAAGTATACGTCTACA | |
| | | AAAGAAGTTTTAGATGCCACTCTTATCCATCAATCCATCACTG | |
| | | GTCTTTATGAAACACGCATTGATTTGAGTCAGCTAGGAGGTG | |
| | | ACGGCACCGGCGGGCCCAAGAAGAAGAGGAAGGTATACCCA | |
| | | TACGATGTTCCTGACTATGCGGGCTATCCCTATGACGTCCCGG | |
| | | ACTATGCAGGATCGTATCCTTATGACGTTCCAGATTACGCTGG | |
| | | ATCCGCCGCTCCGGCAGCTAAGAAAAAGAAACTGGATTTCGA | |
| | | ATCCGGAAAGCCCTATAAATGTCCTGAATGTGGCAAGTCCTT | |
| | | CTCGCGGAGCGACGACCTGACACGGCACCAACGTACGCACAC | |
| | | TGGTGAGAAGCCATACGCGTGTCCTGTCGAGTCCTGTGACCG | |
| | | CCGCTTCAGTCAGAAGGGACACCTGACACGGCACATCCGCAT | |
| | | TCACACAGGGCAAAAACCGTTTCAATGCCGCATCTGCATGAG | |
| | | GAACTTCAGCATCCGTAGCAGCCTGACACGGCACATCCGCAC | |
| | | CCACACAGGAGAAAAGCCCTTCGCCTGTGACATCTGCGGCAG | |
| | | GAAGTTCGCGCTGAGCCACCACCTGACACGGCACACCAAGAT | |
| | | CCACCTCCGTCAGAAAGACCCCGGGTAA | |
| dCas9*_HNH- | gene | ATGGATAAGAAATACTCAATAGGCTTAGCTATCGGCACAAAT | 97 |
| L88 | | AGCGTCGGATGGGCGGTGATCACTGATGAATATAAGGTTCCG | |
| | | TCTAAAAAGTTCAAGGTTCTGGGAAATACAGACCGCCACAGT | |
| | | ATCAAAAAAAATCTTATAGGGGCTCTTTTATTTGACAGTGGA | |
| | | GAGACAGCGGAAGCGACTCGTCTCAAACGGACAGCTCGTAG | |
| | | AAGGTATACACGTCGGAAGAATCGTATTTGTTATCTACAGGA | |
| | | GATTTTTTCAAATGAGATGGCGAAAGTAGATGATAGTTTCTTT | |
| | | CATCGACTTGAAGAGTCTTTTTTGGTGGAAGAAGACAAGAAG | |
| | | CATGAACGTCATCCTATTTTTGGAAATATAGTAGATGAAGTT | |
| | | GCTTATCATGAGAAATATCCAACTATCTATCATCTGCGAAAA | |
| | | AAATTGGTAGATTCTACTGATAAAGCGGATTTGCGCTTAATCT | |
| | | ATTTGGCCTTAGCGCATATGATTAAGTTTCGTGGTCATTTTTT | |
| | | GATTGAGGGAGATTTAAATCCTGATAATAGTGATGTGGACAA | |
| | | ACTATTTATCCAGTTGGTACAAACCTACAATCAATTATTTGAA | |
| | | GAAAACCCTATTAACGCAAGTGGAGTAGATGCTAAAGCGATT | |
| | | CTTTCTGCACGATTGAGTAAATCAAGACGATTAGAAAATCTC | |
| | | ATTGCTCAGCTCCCCGGTGAGAAGAAAAATGGCTTATTTGGG | |
| | | AATCTCATTGCTTTGTCATTGGGTTTGACCCCTAATTTTAAAT | |
| | | CAAATTTTGATTTGGCAGAAGATGCTAAATTACAGCTTTCAA | |
| | | AAGATACTTACGATGATGATTTAGATAATTTATTGGCGCAAA | |
| | | TTGGAGATCAATATGCTGATTTGTTTTTGGCAGCTAAGAATTT | |
| | | ATCAGATGCTATTTTACTTTCAGATATCCTAAGAGTAAATACT | |
| | | GAAATAACTAAGGCTCCCCTATCAGCTTCAATGATTAAACGC | |
| | | TACGATGAACATCATCAAGACTTGACTCTTTTAAAAGCTTTAG | |
| | | TTCGACAACAACTTCCAGAAAAGTATAAAGAAATCTTTTTTG | |
| | | ATCAATCAAAAAACGGATATGCAGGTTATATTGATGGGGGAG | |
| | | CTAGCCAAGAAGAATTTTATAAATTTATCAAACCAATTTTAG | |
| | | AAAAAATGGATGGTACTGAGGAATTATTGGTGAAACTAAATC | |
| | | GTGAAGATTTGCTGCGCAAGCAACGGACCTTTGACAACGGCT | |
| | | CTATTCCCCATCAAATTCACTTGGGTGAGCTGCATGCTATTTT | |
| | | GAGAAGACAAGAAGACTTTTATCCATTTTTAAAAGACAATCG | |
| | | TGAGAAGATTGAAAAAATCTTGACTTTTCGAATTCCTTATTAT | |
| | | GTTGGTCCATTGGCGCGTGGCAATAGTCGTTTTGCATGGATG | |
| | | ACTCGGAAGTCTGAAGAAACAATTACCCCATGGAATTTTGAA | |
| | | GAAGTTGTCGATAAAGGTGCTTCAGCTCAATCATTTATTGAA | |
| | | CGCATGACAAACTTTGATAAAAATCTTCCAAATGAAAAAGTA | |
| | | CTACCAAAACATAGTTTGCTTTATGAGTATTTTACGGTTTATA | |
| | | ACGAATTGACAAAGGTCAAATATGTTACTGAAGGAATGCGAA | |
| | | AACCAGCATTTCTTTCAGGTGAACAGAAGAAAGCCATTGTTG | |
| | | ATTTACTCTTCAAAACAAATCGAAAAGTAACCGTTAAGCAAT | |
| | | TAAAAGAAGATTATTTCAAAAAAATAGAATGTTTTGATAGTG | |
| | | TTGAAATTTCAGGAGTTGAAGATAGATTTAATGCTTCATTAG | |
| | | GTACCTACCATGATTTGCTAAAAATTATTAAAGATAAAGATT | |
| | | TTTTGGATAATGAAGAAAATGAAGATATCTTAGAGGATATTG | |
| | | TTTTAACATTGACCTTATTTGAAGATAGGGAGATGATTGAGG | |
| | | AAAGACTTAAAACATATGCTCACCTCTTTGATGATAAGGTGA | |
| | | TGAAACAGCTTAAACGTCGCCGTTATACTGGTTGGGGACGTT | |
| | | TGTCTCGAAAATTGATTAATGGTATTAGGGATAAGCAATCTG | |
| | | GCAAAACAATATTAGATTTTTTGAAATCAGATGGTTTTGCCA | |
| | | ATCGCAATTTTATGCAGCTGATCCATGATGATAGTTTGACATT | |
| | | TAAAGAAGACATTCAAAAAGCACAAGTGTCTGGACAAGGCG | |
| | | ATAGTTTACATGAACATATTGCAAATTTAGCTGGTAGCCCTGC | |
| | | TATTAAAAAAGGTATTTTACAGACTGTAAAAGTTGTTGATGA | |
| | | ATTGGTCAAAGTAATGGGGCGGCATAAGCCAGAAAATATCGT | |
| | | TATTGAAATGGCACGTGAAAATCAGGGAGGTTCAGGTGGATC | |
| | | GCGCCAATTGGTTGAAACTCGCCAAATCACTAAGCATGTGGC | |
| | | ACAAATTTTGGATAGTCGCATGAATACTAAATACGATGAAAA | |
| | | TGATAAACTTATTCGAGAGGTTAAAGTGATTACCTTAAAATC | |
| | | TAAATTAGTTTCTGACTTCCGAAAAGATTTCCAATTCTATAAA | |
| | | GTACGTGAGATTAACAATTACCATCATGCCCATGATGCGTAT | |
| | | CTAAATGCCGTCGTTGGAACTGCTTTGATTAAGAAATATCCA | |
| | | AAACTTGAATCGGAGTTTGTCTATGGTGATTATAAAGTTTATG | |
| | | ATGTTCGTAAAATGATTGCTAAGTCTGAGCAAGAAATAGGCA | |
| | | AAGCAACCGCAAAATATTTCTTTTACTCTAATATCATGAACTT | |
| | | CTTCAAAACAGAAATTACACTTGCAAATGGAGAGATTCGCAA | |
| | | ACGCCCTCTAATCGAAACTAATGGGGAAACTGGAGAAATTGT | |
| | | CTGGGATAAAGGGCGAGATTTTGCCACAGTGCGCAAAGTATT | |
| | | GTCCATGCCCCAAGTCAATATTGTCAAGAAAACAGAAGTACA | |
| | | GACAGGCGGATTCTCCAAGGAGTCAATTTTACCAAAAAGAAA | |
| | | TTCGGACAAGCTTATTGCTCGTAAAAAAGACTGGGATCCAAA | |
| | | AAAATATGGTGGTTTTGATAGTCCAACGGTAGCTTATTCAGTC | |
| | | CTAGTGGTTGCTAAGGTGGAAAAAGGGAAATCGAAGAAGTT | |
| | | AAAATCCGTTAAAGAGTTACTAGGGATCACAATTATGGAAAG | |
| | | AAGTTCCTTTGAAAAAAATCCGATTGACTTTTTAGAAGCTAA | |
| | | AGGATATAAGGAAGTTAAAAAAGACTTAATCATTAAACTACC | |
| | | TAAATATAGTCTTTTTGAGTTAGAAAACGGTCGTAAACGGAT | |
| | | GCTGGCTAGTGCCGGAGAATTACAAAAAGGAAATGAGCTGG | |
| | | CTCTGCCAAGCAAATATGTGAATTTTTTATATTTAGCTAGTCA | |
| | | TTATGAAAAGTTGAAGGGTAGTCCAGAAGATAACGAACAAA | |
| | | AACAATTGTTTGTGGAGCAGCATAAGCATTATTTAGATGAGA | |
| | | TTATTGAGCAAATCAGTGAATTTTCTAAGCGTGTTATTTTAGC | |
| | | AGATGCCAATTTAGATAAAGTTCTTAGTGCATATAACAAACA | |
| | | TAGAGACAAACCAATACGTGAACAAGCAGAAAATATTATTCA | |
| | | TTTATTTACGTTGACGAATCTTGGAGCTCCCGCTGCTTTTAAA | |
| | | TATTTTGATACAACAATTGATCGTAAAAAGTATACGTCTACA | |
| | | AAAGAAGTTTTAGATGCCACTCTTATCCATCAATCCATCACTG | |
| | | GTCTTTATGAAACACGCATTGATTTGAGTCAGCTAGGAGGTG | |
| | | ACGGCACCGGCGGGCCCAAGAAGAAGAGGAAGGTATACCCA | |
| | | TACGATGTTCCTGACTATGCGGGCTATCCCTATGACGTCCCGG | |
| | | ACTATGCAGGATCGTATCCTTATGACGTTCCAGATTACGCTGG | |
| | | ATCCGCCGCTCCGGCAGCTAAGAAAAAGAAACTGGATTACCC | |
| | | GTATGACGTACCTGATTACGCTGGTTATCCCTATGATGTCCCG | |
| | | GACTACGCTGGCTCGTACCCTTATGATGTACCTGACTACGCTT | |
| | | TCGAATCCGGAAAGCCCTATAAATGTCCTGAATGTGGCAAGT | |
| | | CCTTCTCGCGGAGCGACGACCTGACACGGCACCAACGTACGC | |
| | | ACACTGGTGAGAAGCCATACGCGTGTCCTGTCGAGTCCTGTG | |
| | | ACCGCCGCTTCAGTCAGAAGGGACACCTGACACGGCACATCC | |
| | | GCATTCACACAGGGCAAAAACCGTTTCAATGCCGCATCTGCA | |
| | | TGAGGAACTTCAGCATCCGTAGCAGCCTGACACGGCACATCC | |
| | | GCACCCACACAGGAGAAAAGCCCTTCGCCTGTGACATCTGCG | |
| | | GCAGGAAGTTCGCGCTGAGCCACCACCTGACACGGCACACCA | |
| | | AGATCCACCTCCGTCAGAAAGACCCCGGGTAA | |
| dCas9*_PhlF | gene | ATGGATAAGAAATACTCAATAGGCTTAGCTATCGGCACAAAT | 98 |
| | | AGCGTCGGATGGGCGGTGATCACTGATGAATATAAGGTTCCG | |
| | | TCTAAAAAGTTCAAGGTTCTGGGAAATACAGACCGCCACAGT | |
| | | ATCAAAAAAAATCTTATAGGGGCTCTTTTATTTGACAGTGGA | |
| | | GAGACAGCGGAAGCGACTCGTCTCAAACGGACAGCTCGTAG | |
| | | AAGGTATACACGTCGGAAGAATCGTATTTGTTATCTACAGGA | |
| | | GATTTTTTCAAATGAGATGGCGAAAGTAGATGATAGTTTCTTT | |
| | | CATCGACTTGAAGAGTCTTTTTTGGTGGAAGAAGACAAGAAG | |
| | | CATGAACGTCATCCTATTTTTGGAAATATAGTAGATGAAGTT | |
| | | GCTTATCATGAGAAATATCCAACTATCTATCATCTGCGAAAA | |
| | | AAATTGGTAGATTCTACTGATAAAGCGGATTTGCGCTTAATCT | |
| | | ATTTGGCCTTAGCGCATATGATTAAGTTTCGTGGTCATTTTTT | |
| | | GATTGAGGGAGATTTAAATCCTGATAATAGTGATGTGGACAA | |
| | | ACTATTTATCCAGTTGGTACAAACCTACAATCAATTATTTGAA | |
| | | GAAAACCCTATTAACGCAAGTGGAGTAGATGCTAAAGCGATT | |
| | | CTTTCTGCACGATTGAGTAAATCAAGACGATTAGAAAATCTC | |
| | | ATTGCTCAGCTCCCCGGTGAGAAGAAAAATGGCTTATTTGGG | |
| | | AATCTCATTGCTTTGTCATTGGGTTTGACCCCTAATTTTAAAT | |
| | | CAAATTTTGATTTGGCAGAAGATGCTAAATTACAGCTTTCAA | |
| | | AAGATACTTACGATGATGATTTAGATAATTTATTGGCGCAAA | |
| | | TTGGAGATCAATATGCTGATTTGTTTTTGGCAGCTAAGAATTT | |
| | | ATCAGATGCTATTTTACTTTCAGATATCCTAAGAGTAAATACT | |
| | | GAAATAACTAAGGCTCCCCTATCAGCTTCAATGATTAAACGC | |
| | | TACGATGAACATCATCAAGACTTGACTCTTTTAAAAGCTTTAG | |
| | | TTCGACAACAACTTCCAGAAAAGTATAAAGAAATCTTTTTTG | |
| | | ATCAATCAAAAAACGGATATGCAGGTTATATTGATGGGGGAG | |
| | | CTAGCCAAGAAGAATTTTATAAATTTATCAAACCAATTTTAG | |
| | | AAAAAATGGATGGTACTGAGGAATTATTGGTGAAACTAAATC | |
| | | GTGAAGATTTGCTGCGCAAGCAACGGACCTTTGACAACGGCT | |
| | | CTATTCCCCATCAAATTCACTTGGGTGAGCTGCATGCTATTTT | |
| | | GAGAAGACAAGAAGACTTTTATCCATTTTTAAAAGACAATCG | |
| | | TGAGAAGATTGAAAAAATCTTGACTTTTCGAATTCCTTATTAT | |
| | | GTTGGTCCATTGGCGCGTGGCAATAGTCGTTTTGCATGGATG | |
| | | ACTCGGAAGTCTGAAGAAACAATTACCCCATGGAATTTTGAA | |
| | | GAAGTTGTCGATAAAGGTGCTTCAGCTCAATCATTTATTGAA | |
| | | CGCATGACAAACTTTGATAAAAATCTTCCAAATGAAAAAGTA | |
| | | CTACCAAAACATAGTTTGCTTTATGAGTATTTTACGGTTTATA | |
| | | ACGAATTGACAAAGGTCAAATATGTTACTGAAGGAATGCGAA | |
| | | AACCAGCATTTCTTTCAGGTGAACAGAAGAAAGCCATTGTTG | |
| | | ATTTACTCTTCAAAACAAATCGAAAAGTAACCGTTAAGCAAT | |
| | | TAAAAGAAGATTATTTCAAAAAAATAGAATGTTTTGATAGTG | |
| | | TTGAAATTTCAGGAGTTGAAGATAGATTTAATGCTTCATTAG | |
| | | GTACCTACCATGATTTGCTAAAAATTATTAAAGATAAAGATT | |
| | | TTTTGGATAATGAAGAAAATGAAGATATCTTAGAGGATATTG | |
| | | TTTTAACATTGACCTTATTTGAAGATAGGGAGATGATTGAGG | |
| | | AAAGACTTAAAACATATGCTCACCTCTTTGATGATAAGGTGA | |
| | | TGAAACAGCTTAAACGTCGCCGTTATACTGGTTGGGGACGTT | |
| | | TGTCTCGAAAATTGATTAATGGTATTAGGGATAAGCAATCTG | |
| | | GCAAAACAATATTAGATTTTTTGAAATCAGATGGTTTTGCCA | |
| | | ATCGCAATTTTATGCAGCTGATCCATGATGATAGTTTGACATT | |
| | | TAAAGAAGACATTCAAAAAGCACAAGTGTCTGGACAAGGCG | |
| | | ATAGTTTACATGAACATATTGCAAATTTAGCTGGTAGCCCTGC | |
| | | TATTAAAAAAGGTATTTTACAGACTGTAAAAGTTGTTGATGA | |
| | | ATTGGTCAAAGTAATGGGGCGGCATAAGCCAGAAAATATCGT | |
| | | TATTGAAATGGCACGTGAAAATCAGGGAGGTTCAGGTGGATC | |
| | | GCGCCAATTGGTTGAAACTCGCCAAATCACTAAGCATGTGGC | |
| | | ACAAATTTTGGATAGTCGCATGAATACTAAATACGATGAAAA | |
| | | TGATAAACTTATTCGAGAGGTTAAAGTGATTACCTTAAAATC | |
| | | TAAATTAGTTTCTGACTTCCGAAAAGATTTCCAATTCTATAAA | |
| | | GTACGTGAGATTAACAATTACCATCATGCCCATGATGCGTAT | |
| | | CTAAATGCCGTCGTTGGAACTGCTTTGATTAAGAAATATCCA | |
| | | AAACTTGAATCGGAGTTTGTCTATGGTGATTATAAAGTTTATG | |
| | | ATGTTCGTAAAATGATTGCTAAGTCTGAGCAAGAAATAGGCA | |
| | | AAGCAACCGCAAAATATTTCTTTTACTCTAATATCATGAACTT | |
| | | CTTCAAAACAGAAATTACACTTGCAAATGGAGAGATTCGCAA | |
| | | ACGCCCTCTAATCGAAACTAATGGGGAAACTGGAGAAATTGT | |
| | | CTGGGATAAAGGGCGAGATTTTGCCACAGTGCGCAAAGTATT | |
| | | GTCCATGCCCCAAGTCAATATTGTCAAGAAAACAGAAGTACA | |
| | | GACAGGCGGATTCTCCAAGGAGTCAATTTTACCAAAAAGAAA | |
| | | TTCGGACAAGCTTATTGCTCGTAAAAAAGACTGGGATCCAAA | |
| | | AAAATATGGTGGTTTTGATAGTCCAACGGTAGCTTATTCAGTC | |
| | | CTAGTGGTTGCTAAGGTGGAAAAAGGGAAATCGAAGAAGTT | |
| | | AAAATCCGTTAAAGAGTTACTAGGGATCACAATTATGGAAAG | |
| | | AAGTTCCTTTGAAAAAAATCCGATTGACTTTTTAGAAGCTAA | |
| | | AGGATATAAGGAAGTTAAAAAAGACTTAATCATTAAACTACC | |
| | | TAAATATAGTCTTTTTGAGTTAGAAAACGGTCGTAAACGGAT | |
| | | GCTGGCTAGTGCCGGAGAATTACAAAAAGGAAATGAGCTGG | |
| | | CTCTGCCAAGCAAATATGTGAATTTTTTATATTTAGCTAGTCA | |
| | | TTATGAAAAGTTGAAGGGTAGTCCAGAAGATAACGAACAAA | |
| | | AACAATTGTTTGTGGAGCAGCATAAGCATTATTTAGATGAGA | |
| | | TTATTGAGCAAATCAGTGAATTTTCTAAGCGTGTTATTTTAGC | |
| | | AGATGCCAATTTAGATAAAGTTCTTAGTGCATATAACAAACA | |
| | | TAGAGACAAACCAATACGTGAACAAGCAGAAAATATTATTCA | |
| | | TTTATTTACGTTGACGAATCTTGGAGCTCCCGCTGCTTTTAAA | |
| | | TATTTTGATACAACAATTGATCGTAAAAAGTATACGTCTACA | |
| | | AAAGAAGTTTTAGATGCCACTCTTATCCATCAATCCATCACTG | |
| | | GTCTTTATGAAACACGCATTGATTTGAGTCAGCTAGGAGGTG | |
| | | ACGGCACCGGCGGGCCCAAGAAGAAGAGGAAGGTATACCCA | |
| | | TACGATGTTCCTGACTATGCGGGCTATCCCTATGACGTCCCGG | |
| | | ACTATGCAGGATCGTATCCTTATGACGTTCCAGATTACGCTGG | |
| | | ATCCGCCGCTCCGGCAGCTAAGAAAAAGAAACTGGATTACCC | |
| | | GTATGACGTACCTGATTACGCTGGTTATCCCTATGATGTCCCG | |
| | | GACTACGCTGGCTCGTACCCTTATGATGTACCTGACTACGCTT | |
| | | TCGAATCCGGAGCACGTACCCCGAGCCGTAGCAGCATTGGTA | |
| | | GCCTGCGTAGTCCGCATACCCATAAAGCAATTCTGACCAGCA | |
| | | CCATTGAAATCCTGAAAGAATGTGGTTATAGCGGTCTGAGCA | |
| | | TTGAAAGCGTTGCACGTCGTGCCGGTGCAAGCAAACCGACCA | |
| | | TTTATCGTTGGTGGACCAATAAAGCAGCACTGATTGCCGAAG | |
| | | TGTATGAAAATGAAAGCGAACAGGTGCGTAAATTTCCGGATC | |
| | | TGGGTAGCTTTAAAGCCGATCTGGATTTTCTGCTGCGTAATCT | |
| | | GTGGAAAGTTTGGCGTGAAACCATTTGTGGTGAAGCATTTCG | |
| | | TTGTGTTATTGCAGAAGCACAGCTGGACCCTGCAACCCTGAC | |
| | | CCAGCTGAAAGATCAGTTTATGGAACGTCGTCGTGAGATGCC | |
| | | GAAAAAACTGGTTGAAAATGCCATTAGCAATGGTGAACTGCC | |
| | | GAAAGATACCAATCGTGAACTGCTGCTGGATATGATTTTTGG | |
| | | TTTTTGTTGGTATCGCCTGCTGACCGAACAGCTGACCGTTGAA | |
| | | CAGGATATTGAAGAATTTACCTTCCTGCTGATTAATGGTGTTT | |
| | | GTCCGGGTACACAGCGTTAA | |
| rfp | gene | ATGGCTTCCTCCGAAGACGTTATCAAAGAGTTCATGCGTTTCA | 99 |
| | | AAGTTCGTATGGAAGGTTCCGTTAACGGTCACGAGTTCGAAA | |
| | | TCGAAGGTGAAGGTGAAGGTCGTCCGTACGAAGGTACCCAG | |
| | | ACCGCTAAACTGAAAGTTACCAAAGGTGGTCCGCTGCCGTTC | |
| | | GCTTGGGACATCCTGTCCCCGCAGTTCCAGTACGGTTCCAAA | |
| | | GCTTACGTTAAACACCCGGCTGACATCCCGGACTACCTGAAA | |
| | | CTGTCCTTCCCGGAAGGTTTCAAATGGGAACGTGTTATGAAC | |
| | | TTCGAAGACGGTGGTGTTGTTACCGTTACCCAGGACTCCTCCC | |
| | | TGCAAGACGGTGAGTTCATCTACAAAGTTAAACTGCGTGGTA | |
| | | CCAACTTCCCGTCCGACGGTCCGGTTATGCAGAAAAAAACCA | |
| | | TGGGTTGGGAAGCTTCCACCGAACGTATGTACCCGGAAGACG | |
| | | GTGCTCTGAAAGGTGAAATCAAAATGCGTCTGAAACTGAAAG | |
| | | ACGGTGGTCACTACGACGCTGAAGTTAAAACCACCTACATGG | |
| | | CTAAAAAACCGGTTCAGCTGCCGGGTGCTTACAAAACCGACA | |
| | | TCAAACTGGACATCACCTCCCACAACGAAGACTACACCATCG | |
| | | TTGAACAGTACGAACGTGCTGAAGGTCGTCACTCCACCGGTG | |
| | | CTTAATAA | |
| lacI | gene | ATGAAACCAGTAACGTTATACGATGTCGCAGAGTATGCCGGT | 122 |
| | | GTCTCTTATCAGACCGTTTCCCGCGTGGTGAACCAGGCCAGC | |
| | | CACGTTTCTGCGAAAACGCGGGAAAAAGTGGAAGCGGCGAT | |
| | | GGCGGAGCTGAATTACATTCCCAACCGCGTGGCACAACAACT | |
| | | GGCGGGCAAACAGTCGTTGCTGATTGGCGTTGCCACCTCCAG | |
| | | TCTGGCCCTGCACGCGCCGTCGCAAATTGTCGCGGCGATTAA | |
| | | ATCTCGCGCCGATCAACTGGGTGCCAGCGTGGTGGTGTCGAT | |
| | | GGTAGAACGAAGCGGCGTCGAAGCCTGTAAAGCGGCGGTGC | |
| | | ACAATCTTCTCGCGCAACGCGTCAGTGGGCTGATCATTAACT | |
| | | ATCCGCTGGATGACCAGGATGCCATTGCTGTGGAAGCTGCCT | |
| | | GCACTAATGTTCCGGCGTTATTTCTTGATGTCTCTGACCAGAC | |
| | | ACCCATCAACAGTATTATTTTCTCCCATGAGGACGGTACGCG | |
| | | ACTGGGCGTGGAGCATCTGGTCGCATTGGGTCACCAGCAAAT | |
| | | CGCGCTGTTAGCGGGCCCATTAAGTTCTGTCTCGGCGCGTCTG | |
| | | CGTCTGGCTGGCTGGCATAAATATCTCACTCGCAATCAAATTC | |
| | | AGCCGATAGCGGAACGGGAAGGCGACTGGAGTGCCATGTCC | |
| | | GGTTTTCAACAAACCATGCAAATGCTGAATGAGGGCATCGTT | |
| | | CCCACTGCGATGCTGGTTGCCAACGATCAGATGGCGCTGGGC | |
| | | GCAATGCGCGCCATTACCGAGTCCGGGCTGCGCGTTGGTGCG | |
| | | GATATCTCGGTAGTGGGATACGACGATACCGAAGATAGCTCA | |
| | | TGTTATATCCCGCCGTTAACCACCATCAAACAGGATTTTCGCC | |
| | | TGCTGGGGCAAACCAGCGTGGACCGCTTGCTGCAACTCTCTC | |
| | | AGGGCCAGGCGGTGAAGGGCAATCAGCTGTTGCCAGTCTCAC | |
| | | TGGTGAAAAGAAAAACCACCCTGGCGCCCAATACGCAAACC | |
| | | GCCTCTCCCCGCGCGTTGGCCGATTCATTAATGCAGCTGGCAC | |
| | | GACAGGTTTCCCGACTGGAAAGCGGGCAGTGA | |
| tetR | gene | ATGTCCAGATTAGATAAAAGTAAAGTGATTAACAGCGCATTA | 100 |
| | | GAGCTGCTTAATGAGGTCGGAATCGAAGGTTTAACAACCCGT | |
| | | AAACTCGCCCAGAAGCTAGGTGTAGAGCAGCCTACATTGTAT | |
| | | TGGCATGTAAAAAATAAGCGGGCTTTGCTCGACGCCTTAGCC | |
| | | ATTGAGATGTTAGATAGGCACCATACTCACTTTTGCCCTTTAG | |
| | | AAGGGGAAAGCTGGCAAGATTTTTTACGTAATAACGCTAAAA | |
| | | GTTTTAGATGTGCTTTACTAAGTCATCGCGATGGAGCAAAAG | |
| | | TACATTTAGGTACACGGCCTACAGAAAAACAGTATGAAACTC | |
| | | TCGAAAATCAATTAGCCTTTTTATGCCAACAAGGTTTTTCACT | |
| | | AGAGAATGCATTATATGCACTCAGCGCTGTGGGGCATTTTAC | |
| | | TTTAGGTTGCGTATTGGAAGATCAAGAGCATCAAGTCGCTAA | |
| | | AGAAGAAAGGGAAACACCTACTACTGATAGTATGCCGCCATT | |
| | | ATTACGACAAGCTATCGAATTATTTGATCACCAAGGTGCAGA | |
| | | GCCAGCCTTCTTATTCGGCCTTGAATTGATCATATGCGGATTA | |
| | | GAAAAACAACTTAAATGTGAAAGTGGGTCCTAA | |
| vanR(1) | gene | ATGGACATGCCTCGTATTAAACCGGGTCAGCGTGTTATGATG | 101 |
| | | GCACTGCGTAAAATGATTGCAAGCGGTGAAATCAAAAGTGGT | |
| | | GAACGTATTGCAGAAATTCCGACCGCAGCAGCACTGGGTGTT | |
| | | AGCCGTATGCCGGTTCGTATCGCACTGCGTTCACTGGAACAA | |
| | | GAAGGTCTGGTTGTTCGTCTGGGTGCACGTGGTTATGCAGCC | |
| | | CGTGGTGTTAGCAGCGATCAGATTCGTGATGCAATTGAAGTT | |
| | | CGTGGTGTTCTGGAAGGTTTTGCAGCACGTCGTCTGGCAGAA | |
| | | CGTGGTATGACCGCAGAAACCCATGCACGTTTTGTTGTACTG | |
| | | ATTGCAGAAGGTGAAGCACTGTTTGCAGCCGGTCGCCTGAAT | |
| | | GGTGAAGATCTGGATCGTTATGCCGCATATAATCAGGCATTT | |
| | | CATGATACCCTGGTTAGCGCAGCAGGTAATGGTGCAGTTGAA | |
| | | AGCGCACTGGCACGTAATGGTTTTGAACCGTTTGCAGCAGCC | |
| | | GGTGCACTGGCCCTGGATCTGATGGACCTGTCTGCCGAATAT | |
| | | GAACATCTGCTGGCAGCACATCGTCAGCATCAGGCAGTTCTG | |
| | | GATGCAGTTAGCTGTGGTGATGCCGAAGGTGCAGAACGTATT | |
| | | ATGCGTGATCATGCACTGGCAGCAATTCGTAATGCAAAAGTT | |
| | | TTTGAAGCAGCAGCAAGCGCAGGCGCACCGCTGGGTGCAGC | |
| | | ATGGTCAATTCGTGCAGATTGA | |
| betI(1) | gene | ATGCCGAAACTGGGTATGCAGAGCATTCGTCGTCGTCAGCTG | 102 |
| | | ATTGATGCAACCCTGGAAGCAATTAATGAAGTTGGTATGCAT | |
| | | GATGCAACCATTGCACAGATTGCACGTCGTGCCGGTGTTAGC | |
| | | ACCGGTATTATTAGCCATTATTTCCGCGATAAAAACGGTCTAC | |
| | | TGGAAGCAACCATGCGTGATATTACCAGCCAGCTGCGTGATG | |
| | | CAGTTCTGAATCGTCTGCATGCACTGCCGCAGGGTAGCGCAG | |
| | | AACAGCGTCTGCAGGCAATTGTTGGTGGTAATTTTGATGAAA | |
| | | CCCAGGTTAGCAGCGCAGCAATGAAAGCATGGCTGGCATTTT | |
| | | GGGCAATCAGCATGCATCAGCCGATGCTGTATCGTCTGCAGC | |
| | | AGGTTAGCAGTCGTCGTCTGCTGAGCAATCTGGTTAGCGAAT | |
| | | TTCGTCGTGAACTGCCTCGTGAACAGGCACAAGAGGCAGGTT | |
| | | ATGGTCTGGCAGCACTGATTGATGGTCTGTGGCTGCGTGCAG | |
| | | CACTGAGCGGTAAACCGCTGGATAAAACCCGTGCAAATAGCC | |
| | | TGACCCGTCATTTTATCACCCAGCATCTGCCGACCGATTGA | |
| luxR | gene | ATGAAAAACATAAATGCCGACGACACATACAGAATAATTAAT | 103 |
| | | AAAATTAAAGCTTGTAGAAGCAATAATGATATTAATCAATGC | |
| | | TTATCTGATATGACTAAAATGGTACATTGTGAATATTATTTAC | |
| | | TCGCGATCATTTATCCTCATTCTATGGTTAAATCTGATATTTC | |
| | | AATCCTAGATAATTACCCTAAAAAATGGAGGCAATATTATGA | |
| | | TGACGCTAATTTAATAAAATATGATCCTATAGTAGATTATTCT | |
| | | AACTCCAATCATTCACCAATTAATTGGAATATATTTGAAAAC | |
| | | AATGCTGTAAATAAAAAATCTCCAAATGTAATTAAAGAAGCG | |
| | | AAAACATCAGGTCTTATCACTGGGTTTAGTTTCCCTATTCATA | |
| | | CGGCTAACAATGGCTTCGGAATGCTTAGTTTTGCACATTCAG | |
| | | AAAAAGACAACTATATAGATAGTTTATTTTTACATGCGTGTAT | |
| | | GAACATACCATTAATTGTTCCTTCTCTAGTTGATAATTATCGA | |
| | | AAAATAAATATAGCAAATAATAAATCAAACAACGATTTAACC | |
| | | AAAAGAGAAAAAGAATGTTTAGCGTGGGCATGCGAAGGAAA | |
| | | AAGCTCTTGGGATATTTCAAAAATATTAGGTTGCAGTGAGCG | |
| | | TACTGTCACTTTCCATTTAACCAATGCGCAAATGAAACTCAAT | |
| | | ACAACAAACCGCTGCCAAAGTATTTCTAAAGCAATTTTAACA | |
| | | GGAGCAATTGATTGCCCATACTTTAAAAATTAA | |
| T1 | Terminator | AAAAAAAAAAAAGGCCTCCCAAATCGGGGGGCCTTTTTTATT | 104 |
| | | GATAACAAAA | |
| T2 | Terminator | CTCGGTACCAAATTCCAGAAAAGAGACGCTTAACAGCGTCTT | 105 |
| | | TTTTCGTTTTGGTCC | |
| T3 | Terminator | CCAATTATTGAAGGCCGCTAACGCGGCCTTTTTTTGTTTCTGG | 106 |
| | | TCTCCC | |
| T4 | Terminator | CCAATTATTGAAGGCCTCCCAAATCGGGGGGCCTTTTTTATTG | 107 |
| | | ATAACAAAA | |
| T5 | Terminator | CTCGGTACCAAAAAAAAAAAAAAAGACGCTGAAAAGCGTCT | 108 |
| | | TTTTTCGTTTTGGTCC | |
| T6 | Terminator | CTCGGTACCAAAAAAAAAAAAAAAGACGCTGAAAAGCGTCT | 109 |
| | | TTTTTTTTTTTGGTCC | |
| T7 | Terminator | CTCGGTACCAAACCAATTATTGAAGACGCTGAAAAGCGTCTT | 110 |
| | | TTTTCGTTTTGGTCC | |
| T8 | Terminator | CTCGGTACCAAATTCCAGAAAAGAGACGCTTTTAGAGCGTCT | 111 |
| | | TTTTTCGTTTTGGTCC | |
| T9 | Terminator | CTCGGTACCAAATTCCAGAAAAGAGACGCTGAAAAGCGTCTT | 112 |
| | | TTTTTTTTTTGGTCC | |
| T10 | Terminator | GACGAACAATAAGGCCTCCCTAACGGGGGGCCTTTTTTATTG | 113 |
| | | ATAACAAAA | |
| T11 | Terminator | GACGAACAATAAGGCCTCCCGAAAGGGGGGCCTTTTTTATTG | 114 |
| | | ATAACAAAA | |
| T12 | Terminator | TCTAACTAAAAACACCCTAACGGGTGTTTTTTTGTTTCTGGTC | 115 |
| | | TGCC | |
| T13 | Terminator | CCAATTATTGAACACCCTTCGGGGTGTTTTTTTGTTTCTGGTCT | 116 |
| | | CCC | |
| T14 | Terminator | CCAATTATTGAAGACGCTTAACAGCGTCTTTTTTTGTTTCTGG | 117 |
| | | TCTCCC | |
| T15 | Terminator | TTTTCGAAAAAACACCCTAACGGGTGTTTTTTTGTTTCTGGTC | 118 |
| | | TCCC | |
| T16 | Terminator | CTCGGTACCAAATCTAACTAAAAAGACGCTGAAAAGCGTCTT | 119 |
| | | TTTTCGTTTTGGTCC | |
| L3S2P55 | Terminator | CTCGGTACCAAAGACGAACAATAAGACGCTGAAAAGCGTCTT | 120 |
| | | TTTTCGTTTTGGTCC | |
| L3S2P53 | Terminator | CTCGGTACCAAACCAATTATTGAAGACGCTGAAAAGCGTCTT | 121 |
| | | TTTTCGTTTTGGTCC | |
| L3S2P11 | Terminator | CTCGGTACCAAATTCCAGAAAAGAGACGCTTTCGAGCGTCTT | 123 |
| | | TTTTCGTTTTGGTCC | |
| L3S2P44 | Terminator | CTCGGTACCAAACCAATTATTGAAGACGCTGAAAAGCGTCTT | 124 |
| | | TTTTTGTTTCGGTCC | |
| ECK1200 | Terminator | GGAAACACAGAAAAAAGCCCGCACCTGACAGTGCGGGCTTTT | 125 |
| 33737 | | TTTTTCGACCAAAGG | |
| B0010* | Terminator | CCAGGCATCAAATAAAACGAAAGGCTCAGTCGAAAGACTGG | 126 |
| | | GCCTTTCGTTTTATCTGTTGTTTGTCGGTGAACGCTCTCTACTA | |
| | | GAGTCACACTGGCTCACCTTCGGGTGGGCCTTTCTGCGTTTAT | |
| | | A | |

One goal of this study was to evaluate a maximum number of sgRNAs that can be used together. Therefore, the system was tuned to minimize the expression level of each sgRNA to the point where they are as low as possible but still could minimally function as a NOT gate. In accordance with this approach, the constitutive promoter (pCon) was selected such that each sgRNA yields ˜10-fold repression when measured in the context of the N16 construct (FIG. 12). In essence, this maximizes the number of sgRNAs that can be used simultaneously, thus representing an upper limit. Toxicity of expressing sgRNAs was also studied and only a slight decrease in the growth of E. coli cells was observed as more sgRNAs were simultaneously expressed (FIG. 13).

The impact on the sgRNA9 gate was measured as a function of the number of additional sgRNAs co-expressed (FIG. 3B). The additional sgRNAs do not bind to any DNA sequences in the system because their cognate promoters are not included. This response was compared for both dCas9 and dCas9*_PhlF expressed to the maximal level prior to observing a growth defect (0.7 and 2.5 ng/ml aTc, respectively). In both cases, there is a significant decline in repression even with the first few additional sgRNAs. The slope is steeper for dCas9 and the response falls below 10-fold after 7 more sgRNAs are co-expressed, while for dCas9*_PhlF this increases to 14 sgRNAs.

Discussion.

The original uses intended for Cas9 and dCas9 have different constraints than those required for genetic circuits. Genome editing and knockdown experiments only require transient and low levels of expression for activity. These applications benefit from the capability of sgRNA to be designed to target essentially any region of the genome and this programmability could be very useful for building out sets of orthogonal regulators for genetic circuits. However, integrating a circuit into an application is more complicated, for example to produce a chemical product in a fermenter or integrate information in the human gut (Lian J., et al., Nat. Commun., 2017 Nov. 22; 8(1): 1688; Cress B. F., et al., Nucleic Acids Res., 2016 May 19; 44(9): 4472-85; Mimee M., et al., Cell Syst., 2015 Jul. 29; 1(1): 62-71; Fernandez-Rodriguez J., et al., Nat. Chem. Biol., 2017 July; 13(7): 706-8; Brophy J. A. N. and Voigt, C. A., Nat. Methods, 2014. 11(5): 508-20). For these purposes, a circuit cannot reduce growth or require significant cellular resources or energy to function. One of these problems has been solved, as described herein, where the growth impact of dCas9 is greatly reduced by increasing the required DNA sequence to which it binds by swapping a 3 bp PAM site for a 30 bp PhlF operator. This allows the expression of dCas9*_PhlF to be increased to ˜10⁴ copies per cell, which is just about as high as one can expect to push the expression of a large protein in E. coli (Milo R. and Phillips R., Garland Science, 2015).

Repetitive sequences shared between gates is another challenge that must be solved before large sgRNA circuits can be built based on dCas9*_PhlF. The shared sequences can lead to genetic instability due to homologous recombination (Lou C., et al., Nat. Biotechnol., 2012 November; 30(11): 1137-42; Sleight S. C. and Sauro H. M., ACS Synth. Biol., 2013 Sep. 20; 2(9): 519-28). All of the sgRNA-based gates share the identical 83 bp tracrRNA sequences, and the output promoters share the identical 30 bp PhlF operator (FIG. 14). In addition, converting the NOT gates to NOR gates requires either duplicating the sgRNA or using a ribozyme to cleave 5′-UTR generated by two upstream promoters in series (Nielsen A. A., et al., Science, 2016. 352(6281): aac7341; Nielsen A. A. and Voigt C. A., Mol. Syst. Biol., 2014. 10(763): 1-11; Gander M. W., et al., Nat. Commun., 2017 May 25; 8: 15459). Both of these approaches lead to longer regions of repeated DNA. Stabilizing circuits would require sequence diversification and the creation of part libraries (e.g., of ribozymes) with diverse sequences, approaches that have been applied previously (Chen Y. J., et al., Nat. Methods, 2013 July; 10(7): 659-64; vett S. T., et al., Genetics, 2002 March; 160(3): 851-59).

However, before undertaking this effort, it is important to consider whether the concept makes sense. The pool of dCas9*_PhlF would need to be maintained at a constant ˜10⁴ molecules irrespective of the number of active gates. Our experimental data and model show that this can support about 15 sgRNA-based gates (Methods section). This is about on par with the number of available protein-based gates and is a harsh limitation to the huge number of potential gates considering sgRNA programmability alone (estimated to be ˜10⁷ sgRNA-promoter pairs) (Nielsen A. A. and Voigt C. A., Mol. Syst. Biol., 2014. 10(763): 1-11). The retroactivity due to having to share the dCas9*_PhlF resource also changes as each additional sgRNAs is added to the system. When designing circuits, a mathematical model would have to be used to mitigate this complexity. Thus, the benefit of sgRNA-based gates, even when the dCas9 toxicity is solved, is not a scale-up in size, although there may be other benefits for certain scenarios.

One such scenario may be in eukaryotes where using dCas9-based gates have an advantage (Li Y., et al., Nat. Chem. Biol., 2015 March; 11(3): 207-13; Gander M. W., et al., Nat. Commun., 2017 May 25; 8: 15459; Nissim L., et al., Cell, 2017 Nov. 16; 171(5): 1138-50). The lack of translation at the gate level means that that circuit function can be entirely localized to the nucleus (once a dCas9 pool has been imported), thus avoiding the capping and export of the mRNA and importing of each protein-based repressor. Another may be for organisms where for which the circuit needs to be carried at low copy and the design of high-expression promoters remains elusive (Mimee M., et al., Cell Syst., 2015 Jul. 29; 1(1): 62-71).

A false concept is that sgRNA gates require less cellular resources because they do not require translation to function. While each gate only requires a new sgRNA to be transcribed, for it to be functional it needs a dCas9*_PhlF to form a complex that represses the output promoter. The binding of sgRNA to dCas9 is very tight (K_{d}=10 pM) (Wright A. V., et al., Proc. Natl. Acad. Sci. USA, 2015. 112(10): p. 2984-89) and dCas9 binds tightly to DNA (K_{d}=1 nM) (Sternberg S. H., et al., Nature, 2014. 507(7490): 62-67; Richardson C. D., et al., Nat. Biotechnol., 2016 March; 34(3): 339-44; Josephs E. A., et al., Nucleic Acids Res., 2015 Oct. 15; 43(18): 8924-41), requiring DNA replication machinery for removal during division (Jones D. L., et al., Science, 2017 Sep. 29; 357(6358): 1420-24). Therefore, it is likely that recycling of the pool will be low (reuse of dCas9 after dissociating from a previous sgRNA). This makes the cost of each dCas9*_PhlF:sgRNA “repressor” high when compared to a protein-based repressor (e.g., TetR). Putting it in terms of ATP consumption, an estimation is that the former requires ˜6000 ATP/repressor and the latter ˜600 ATP/repressor (Methods).

The sharing of a resource is a common feature of cells, including natural regulatory networks (Cookson N. A., et al., Mol. Syst. Biol., 2011 Dec. 20; 7:561; Mishra D., et al., Nat. Biotechnol., 2014 December; 32(12): 1268-75). One example are sigma factors, turned on in response to different cellular needs, that all must share core RNA polymerase to initiate transcription from a promoter (Gruber T. M. and Gross C. A., Annu. Rev. Microbiol., 2003; 57: 441-66). If multiple sigma factors were co-expressed, this would draw down the core resource. It has been shown that B. subtilis has an innovative solution: each sigma factor is expressed as an independent pulse and the pulsing time is changed with respect to need, as opposed to the expression level (Park J., et al., Cell Syst. 2018 Feb. 28; 6(2): 216-29). In the natural network, this is achieved with feedback loops of a complexity still elusive to achieve in engineered systems. Still, it may be a solution to the circuit limitations of dCas9 as well as other similar problems in the field (Cookson N. A., et al., Mol. Syst. Biol., 2011 Dec. 20; 7:561; Segall-Shapiro T. H., et al., Mol. Syst. Biol., 2014 Jul. 30; 10: 742). Until then, our results point to the difficulty of using a genetic circuit paradigm that requires a shared (and expensive) non-recyclable resource in bacteria. This work highlights the need to develop theoretical and experimental frameworks to quantify the cellular impact of introducing systems into cells, prior to performing experiments, in order to rationally guide design decisions.

### REFERENCES

1. Barrangou R., Fremaux C., Deveau H., Richards M., Boyaval P., Moineau S., Romero D. A., and Horvath P., CRISPR provides acquired resistance against viruses in prokaryotes. Science, 2007 Mar. 23; 315(5819): 1709-12.
2. Bikard D., Jiang W., Samai P., Hochschild A., Zhang F., and Marraffini L. A., Programmable repression and activation of bacterial gene expression using an engineered CRISPR-Cas system. Nucleic Acids Res., 2013 August; 41(15): 7429-37.
3. Blattner F. R., Plunkett G. 3^{rd}, Bloch C. A., Perna N. T., Burland V., Riley M., Collado-Vides J., Glasner J. D., Rode C. K., Mayhew G. F., Gregor J., Davis N. W., Kirkpatrick H. A., Goeden M. A., Rosen D. J., Mau B., and Shao Y., The complete genome sequence of Escherichia coli K-12. Science, 1997 Sep. 5; 277(5331): 1453-62.
4. Bolukbasi M. F., Gupta A., Oikemus S., Den A. G., Garber M., Brodsky M. H., Zhu L. J., and Wolfe S. A., DNA-binding-domain fusions enhance the targeting range and precision of Cas9. Nat. Methods, 2015 December; 12(12): 1150-56.
5. Brewster R. C., Weinert F. M., Garcia H. G., Song D., Rydefelt M., and Phillips R., The transcription factor titration effect dictates level of gene expression. Cell, 2014 March; 156(6): 1312-23.
6. Brophy J. A. N. and Voigt, C. A., Principles of genetic circuit design. Nat. Methods, 2014. 11(5): 508-20.
7. Ceroni F., Boo A., Furini S., Gorochowski T. E., Ladak Y. N., Awan A. R., Gilbert C., Stan G. B., and Ellis T., Burden-driven feedback control of gene expression. Nat. Methods, 2018 May; 15(5): 387-93.
8. Chen B., Gilbert L. A., Cimini B. A., Schnitzbauer J., Zhang W., Li G. W., Park J., Blackburn E. H., Weissman J. S., Qi L. S., and Huang B., Dynamic imaging of genomic loci in living human cells by an optimized CRISPR/Cas system. Cell, 2013. 155(7): 1479-91.
9. Chen P. Y., Qian Y., and Del Vecchio D., A model for resource competition in CRISPR-mediated gene repression. bioRxiv, 2018 Feb. 4: doi.org/10.1101/266015.
10. Chen Y. J., Liu P., Nielsen A. A., Brophy J. A., Clancy K., Peterson T., Voigt C. A., Characterization of 582 natural and synthetic terminators and quantification of their design constraints. Nat. Methods, 2013 July; 10(7): 659-64.
11. Cho S., Choe D., Lee E., Kim S. C., Palsson B., and Cho B. K., High-level dCas9 expression induces abnormal cell morphology in Escherichia coli. ACS Synth. Biol., 2018 Apr. 20; 7(4): 1085-94.
12. Cong L., Ran F. A., Cox D., Lin S., Barretto R., Habib N., Hsu P. D., Wu X., Jiang W., Marraffini L. A., and Zhang F., Multiplex genome engineering using CRISPR/Cas systems. Science, 2013. 339(6121): 819-23.
13. Cookson N. A., Mather W. H., Danino T., Mondragon-Palomino O., Williams R. J., Tsimring L. S., and Hasty J., Queueing up for enzymatic processing: correlated signaling through coupled degradation. Mol. Syst. Biol., 2011 Dec. 20; 7:561.
14. Cress B. F., Jones J. A., Kim D. C., Leitz Q. D., Englaender J. A., Collins S. M., Linhardt R. J., and Koffas M. A., Rapid generation of CRISPR/dCas9-regulated, orthogonally repressible hybrid T7-lac promoters for modular, tuneable control of metabolic pathway fluxes in Escherichia coli. Nucleic Acids Res., 2016 May 19; 44(9): 4472-85.
15. Del Vecchio D., Ninfa A. J. and Sontag E. D., Modular cell biology: retroactivity and insulation. Mol. Syst. Biol., 2008. 4(161): 1-16.
16. Deltcheva E., Chylinski K., Sharma C. M., Conzales K., Chao Y., Pirzada Z. A., Eckert M. R., Vogel J., and Charpentier E., CRISPR RNA maturation by trans-encoded small RNA and host factor RNase III. Nature, 2011 Mar. 31; 471(7340): 602-7.
17. Didovyk A., Borek B., Hasty J., and Tsimring L., Orthogonal modular gene repression in Escherichia coli using engineered CRISPR/Cas9. ACS Synth. Biol., 2016 Jan. 15; 5(1): 81-8.
18. Elowitz M. B. and Leibler S., A synthetic oscillatory network of transcriptional regulators. Nature, 2000. 403(6767): 335-38.
19. Fernandez-Rodriguez J., Moser F., Song M., and Voigt C. A., Engineering RGB color vision into Escherichia coli. Nat. Chem. Biol., 2017 July; 13(7): 706-8.
20. Ferrell J. E. Jr and Ha S. H., Ultrasensitivity part III: cascades, bistable switches, and oscillators. Trends Biochem. Sci., 2014 December; 39(12): 612-8.
21. Fu Y., Sander J. D., Reyon D., Cascio V. M. and Joung J. K., Improving CRISPR-Cas nuclease specificity using truncated guide RNAs. Nat. Biotechnol., 2014. 32(3): 279-84.
22. Gaber R., Lear T., Majerle A., Ster B., Dobnikar A., Bencina M., and Jerala R., Designable DNA-binding domains enable construction of logic circuits in mammalian cells. Nat. Chem. Biol., 2014 March; 10(3): 203-8.
23. Gao Y., Xiong X., Wong S., Charles E. J., Lim W. A., and Qi L. S., Complex transcriptional modulation with orthogonal and inducible dCas9 regulators. Nat. Methods, 2016 December; 13(12): 1043-49.
24. Gander M. W., Vrana J. D., Voje W. E., Carothers J. M., and Klavins E., Digital logic circuits in yeast with CRISPR-dCas9 NOR gates. Nat. Commun., 2017 May 25; 8: 15459.
25. Gardner T. S., Cantor C. R., and Collins J. J., Construction of a genetic toggle switch in Escherichia coli. Nature, 2000 Jan. 20; 403(6767): 339-42.
26. Garg A., Lohmueller J. J., Silver P. A., and Armel T. Z., Engineering synthetic TAL effectors with orthogonal target sites. Nucleic Acids Res., 2012 August; 40(15): 7584-95.
27. Gasiunas G., Barrangou R., Horvath P., and Siksnys V., Cas9-crRNA ribonucleoprotein complex mediates specific DNA cleavage for adaptive immunity in bacteria. Proc. Natl. Acad. Sci. USA, 2012 Sep. 25; 109(39): 15539-40.
28. Guilinger J. P., Thompson D. B. and Liu D. R., Fusion of catalytically inactive Cas9 to FokI nuclease improves the specificity of genome modification. Nat. Biotechnol., 2014. 32(6): 577-82.
29. Gruber T. M. and Gross C. A., Multiple sigma subunits and the partitioning of bacterial transcription space. Annu. Rev. Microbiol., 2003; 57: 441-66.
30. Holowko M. B., Wang H., Jayaraman P., and Poh C. L., Biosensing Vibrio cholerae with genetically engineered Escherichia coli. ACS Synth. Biol., 2016 Nov. 18; 5(11): 1275-83.
31. Hooshangi S., Thiberge S., and Weiss R., Ultrasensitivity and noise propagation in a synthetic transcriptional cascade. Proc. Natl. Acad. Sci. USA, 2005 Mar. 8; 102(10): 3581-86.
32. Hsu P. D., Scott D. A., Weinstein J. A., Ran F. A., Konermann S., Agarwala V., Li Y., Fine E. J., Wu X., Shalem O., Cradick T. J., Marraffini L. A., Bao G., and Zhang F., DNA targeting specificity of RNA-guided Cas9 nucleases. Nat. Biotechnol., 2013. 31(9): 827-32.
33. Jayanthi S., Nilgiriwala K. S., and Del Vecchio D., Retroactivity controls the temporal dynamics of gene transcription. ACS Synth. Biol., 2-13 Aug. 16; 2(8): 431-41.
34. Jinek M., Chylinski K., Fonfara I., Hauer M., Doudna J. A., and Charpentier E., A programmable dual-RNA-guided DNA endonuclease in adaptive bacterial immunity. Science, 2012. 337(6096): 816-821.
35. Jones D. L., Leroy P., Unoson C., Fange D., Curic V., Lawson M. J., and Elf J., Kinetics of dCas9 target search in Escherichia coli. Science, 2017 Sep. 29; 357(6358): 1420-24.
36. Josephs E. A., Kocak D. D., Fitzgibbon C. J., McMenemy J., Gersbach C. A., and Marszalek P. E., Structure and specificity of the RNA-guided endonuclease Cas9 during DNA interrogation, target binding and cleavage. Nucleic Acids Res., 2015 Oct. 15; 43(18): 8924-41.
37. Kaleta C., Schauble S., Rinas U., and Schuster S., Metabolic costs of amino acid and protein production in Escherichia coli. Biotechnol. J., 2013 September; 8(9): 1105-14.
38. Khalil A. S. and Collins J. J., Synthetic biology: applications come of age. Nat. Rev. Genet., 2010 May; 11(5): 367-79.
39. Kiani S., Beal J., Ebrahimkhani M. R., Huh J., Hall R. N., Xie Z., Li Y., and Weiss R., CRISPR transcriptional repression devices and layered circuits in mammalian cells. Nat. Methods, 2014 July; 11(7): 723-6.
40. Kim D., Bae S., Park J., Kim E., Kim S., Yu H. R., Hwang J., Kim J. I., and Kim J. S., Digenome-seq: genome-wide profiling of CRISPR-Cas9 off-target effects in human cells. Nat. Methods, 2015. 12(3): 237-43.
41. Kleinstiver B. P., Prew M. S., Tsai S. Q., Topkar V. V., Nguyen N. T., Zheng Z., Gonzales A. P., Li Z., Peterson R. T., Yeh J. R., Aryee M. J., and Joung J. K., Engineered CRISPR-Cas9 nucleases with altered PAM specificities. Nature, 2015. 523(7561): 481-85.
42. Mali P., Aach J., Stranges P. B., Esvelt K. M., Moosburner M., Kosuri S., Yang L, and Church G. M., CAS9 transcriptional activators for target specificity screening and paired nickases for cooperative genome engineering. Nat. Biotechnol., 2013. 31(9): 833-38.
43. Lee Y. J., Hoynes-O'Connor A., Leong M. C., and Moon T. S., Programmable control of bacterial gene expression with the combined CRISPR and antisense RNA system. Nucleic Acids Res., 2016 Mar. 18; 44(5): 2462-73.
44. Li Y., Jian Y., Liao W., Li Z., Weiss R., and Xie Z., Modular construction of mammalian gene circuits using TALE transcriptional repressors. Nat. Chem. Biol., 2015 March; 11(3): 207-13.
45. Lian J., HamediRad M., Hu S., and Zhao H., Combinatorial metabolic engineering using an orthogonal tri-functional CRISPR system. Nat. Commun., 2017 Nov. 22; 8(1): 1688.
46. Lovett S. T., Hurley R. L., Sutera V. A., Aubuchon R. H., and Lebedeva M. A., Crossing over between regions of limited homology in Escherichia coli: RecA-dependent and RecA-independent pathways. Genetics, 2002 March; 160(3): 851-59.
47. Lou C., Stanton B., Chen Y. J., Munsky B., and Voigt C. A., Ribozyme-based insulator parts buffer synthetic circuits from genetic context. Nat. Biotechnol., 2012 November; 30(11): 1137-42.
48. Lynch M. and Marinov G. K., The bioenergetic costs of a gene. Proc. Natl. Acad. Sci. USA, 2015 Dec. 22; 112(51): 15690-5.
49. Mali P., Yang L., Esvelt K. M., Aach J., Guell M., DiCario J. E., Norville J. E., and Church G. M., RNA-guided human genome engineering via Cas9. Science, 2013. 339(6121): 823-26.
50. Meyer A. J., Segall-Shapiro T. H., and Voigt C. A., Marionette: E. coli containing 12 highly-optimized small molecule sensors. bioRxiv., 2018 Apr. 10: doi.org/10.1101/285866.
51. Milo R. and Phillips R., Cell biology by the numbers. Garland Science, 2015.
52. Mimee M., Tucker A. C., Voigt C. A., and Lu T. K., Programming a human commensal bacterium, Bacteroides thetaiotaomicron, to sense and respond to stimuli in the murine gut microbiota. Cell Syst., 2015 Jul. 29; 1(1): 62-71.
53. Mishra D., Rivera P. M., Lin A., Del Vecchio D., and Weiss R., A load driver device for engineering modularity in biological networks. Nat. Biotechnol., 2014 December; 32(12): 1268-75.
54. Nielsen A. A. and Voigt C. A., Multi-input CRISPR/Cas genetic circuits that interface host regulatory networks. Mol. Syst. Biol., 2014. 10(763): 1-11.
55. Nielsen A. A., Der B. S., Shin J., Vaidyanathan P., Paralanov V., Strychalski E. A., Ross D., Densmore D., and Voigt C. A., Genetic circuit design automation. Science, 2016. 352(6281): aac7341.
56. Nielsen A. A., Segall-Shapiro T. H., and Voigt C. A., Advances in genetic circuit design: novel biochemistries, deep part mining, and precision gene expression. Curr. Opin. Chem. Biol., 2013 December; 17(6): 878-92.
57. Nissim L., Wu M. R., Pery E., Binder-Nissim A., Susuki H. I., Stupp D., Wehrspaun C., Tabach Y., Sharp P. A., and Lu T. K., Synthetic RNA-based immunomodulatory gene circuits for cancer immunotherapy. Cell, 2017 Nov. 16; 171(5): 1138-50.
58. Nihongaki Y., Kawano F., Nakajima T. and Sato M., Photoactivatable CRISPR-Cas9 for optogenetic genome editing. Nat. Biotechnol., 2015. 33(7): 755-60.
59. Park J., Dies M., Lin Y., Hormoz S., Smith-Unna S. E., Quinodoz S., Hernandez-Jimenez M. J., Garcia-Ojalvo J., Lock J. C. W., and Elowitz M. B., Molecular time sharing through dynamic pulsing in single cells. Cell Syst. 2018 Feb. 28; 6(2): 216-29.
60. Pasini M., Fernandez-Castane A., Jaramillo A., de Mas C., Caminal G., and Ferrer P., Using promoter libraries to reduce metabolic burden due to plasmid-encoded proteins in recombinant Escherichia coli. N. Biotechnol. 2016 Jan. 25; 33(1): 78-90.
61. Peters J. M., Silvis M. R., Zhao D., Hawkins J. S., Gross C. A., and Qi L. S., Bacterial CRISPR: accomplishments and prospects. Curr. Opin. Microbiol., 2015 October; 27: 121-26.
62. Purnick P. E. and Weiss R., The second wave of synthetic biology: from modules to systems. Nat. Rev. Mol. Cell. Biol., 2009 June; 10(6): 410-22.
63. Qi Lei S., Larson M. H., Gilbert L. A., Doudna J. A., Weissman J. S., Arkin A. P., and Lim W. A., Repurposing CRISPR as an RNA-guided platform for sequence-specific control of gene expression. Cell, 2013. 152(5): 1173-83.
64. Qian Y., Huang H. H., Jimenez J. I., and Del Vecchio D., Resource competition shapes the response of genetic circuits. ACS Synth. Biol., 2017 Jul. 21; 6(7): 1263-72.
65. Richardson C. D., Ray G. J., DeWitt M. A., Curie G. L., and Corn J. E., Enhancing homology-directed genome editing by catalytically active and inactive CRISPR-Cas9 using asymmetric donor DNA. Nat. Biotechnol., 2016 March; 34(3): 339-44.
66. Rock J. M., Hopkins F. F., Chavez A., Diallo M., Chase M. R., Gerrick E. R., Pritchard J. R., Church G. M., Rubin E. J., Sassetti C. M., Schnappinger D., and Fortune S. M., Programmable transcriptional repression in mycobacteria using an orthogonal CRISPR interference platform. Nat. Microbiol., 2017. 2(16274): 1-9.
67. Segall-Shapiro T. H., Meyer A. J., Ellington A. D., Sontag E. D., and Voigt C. A. A ‘resource allocator’ for transcription based on a highly fragmented T7 RNA polymerase. Mol. Syst. Biol., 2014 Jul. 30; 10: 742.
68. Slaymaker I. M., Gao L., Zetsche B., Scott D. A., Yan W. X., and Zhang F., Rationally engineered Cas9 nucleases with improved specificity. Science, 2016. 351(6268): 84-88.
69. Sleight S. C. and Sauro H. M., Visualization of evolutionary stability dynamics and competitive fitness of Escherichia coli engineered with randomized multigene circuits. ACS Synth. Biol., 2013 Sep. 20; 2(9): 519-28.
70. Stanton B. C., Nielsen A. A., Tamsir A., Clancy K., Peterson T., and Voigt C. A., Genomic mining of prokaryotic repressors for orthogonal logic gates. Nat. Chem. Biol., 2014. 10(2): 99-105.
71. Sternberg S. H., LaFrance B., Kaplan M. and Doudna J. A., Conformational control of DNA target cleavage by CRISPR-Cas9. Nature, 2015. 527(7576): 110-13.
72. Sternberg S. H., Redding S., Jinek M., Green E. C. and Doudna J. A., DNA interrogation by the CRISPR RNA-guided endonuclease Cas9. Nature, 2014. 507(7490): 62-67.
73. Strogatz S. H., Nonlinear dynamics and chaos: with applications to physics, biology, chemistry, and engineering. Hachette UK, 2014.
74. Tamsir A., Tabor J. J. and Voigt C. A., Robust multicellular computing using genetically encoded NOR gates and chemical ‘wires’. Nature, 2011. 469(7329): 212-15.
75. Tzur A., Moore J. K., Jorgensen P., Shapiro H. M., and Kirschner M. W., Optimizing optical flow cytometry for cell volume-based sorting and analysis. PloS One, 2011 Jan. 20; 6(1): e16053.
76. Weinberg B. H., Pham N. T. H., Caraballo L. D., Lozanoski T., Engel A., Bhatia S., and Wong W. W., Large-scale design of robust genetic circuits with multiple inputs and outputs for mammalian cells. Nat. Biotechnol., 2017 May; 35(5): 453-62.
77. Wright A. V., Sternberg S. H., Taylor D. W., Staahl B. T., Bardales J. A., Kornfeld J. E., and Doudna J. A., Rational design of a split-Cas9 enzyme complex. Proc. Natl. Acad. Sci. USA, 2015. 112(10): 2984-89.
78. Yokobayashi Y., Weiss R., and Arnold F. H., Directed evolution of a genetic circuit. Proc. Natl. Acad. Sci. USA, 2002 Dec. 24; 99(26): 16587-91.
79. Zetsche B., Volz S. E. and Zhang F., A Split-Cas9 architecture for inducible genome editing and transcription modulation. Nat. Biotechnol., 2015. 33(2): 139-42.
80. Zhang Y., Ge X., Yang F., Zhang L., Zheng J., Tan X., Jin Z. B., Qu J., and Gu F., Comparison of non-canonical PAMs for CRISPR/Cas9-mediated DNA cleavage in human cells. Sci. Rep., 2014. 4(5405): 1-5.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

1. A genetic circuit comprising a single polynucleotide or a combination of polynucleotides, wherein the single polynucleotide or the combination of polynucleotides encode:
(a) at least one fusion protein comprising a catalytically-inactive CRISPR/Cas protein fused to a transcription factor, wherein the catalytically-inactive CRISPR/Cas protein comprises a mutated PAM domain and a mutated or absent HNH domain,
(b) at least one small guide RNA, and
(c) at least one output sequence whose expression is operably linked to an output promoter, wherein the output promoter comprises a transcription factor operator and a cognate promoter comprising an sgRNA target site and, optionally, a PAM site.

2. The genetic circuit of claim 1, wherein the catalytically-inactive CRISPR/Cas protein of the at least one fusion protein also comprises a functional RuvC domain.

3. The genetic circuit of claim 1, wherein the mutation of the HNH domain of the catalytically-inactive CRISPR/Cas protein of the at least one fusion protein consists of the deletion of the entire domain and its replacement by an amino acid linker sequence.

4. The genetic circuit of claim 1, wherein the catalytically-inactive CRISPR/Cas protein of the at least one fusion protein consists of amino acids 1 to 1368 of Cas9, wherein the Cas9 amino acid sequence contains D10A and R1335K mutations and the Cas9 amino acids 768 to 919 are replaced by a GGSGGS (SEQ ID NO: 127) amino acid linker sequence.

5. (canceled)

6. The genetic circuit of claim 1, wherein the transcription factor of the at least one fusion protein is selected from the group consisting of PhlF, BM3R1, and a ZFP protein.

7.-8. (canceled)

9. The genetic circuit of claim 1, wherein the transcription factor of the at least one fusion protein activates the expression of the at least one output sequence.

10. The genetic circuit of claim 1, wherein:
the transcription factor operator and the cognate promoter of the output promoter that is operably linked to the at least one output sequence are on the same DNA strand;
the transcription factor operator and the cognate promoter of the output promoter that is operably linked to the at least one output sequence are on complementary DNA strands;
the catalytically-inactive CRISPR/Cas protein of the at least one fusion protein is fused to the transcription factor of the at least one fusion protein with a C-terminal polypeptide bond; or
the catalytically-inactive CRISPR/Cas protein of the at least one fusion protein is fused to the transcription factor of the at least one fusion protein with an N-terminal polypeptide bond.

11. (canceled)

12. The genetic circuit of claim 1, wherein the transcription factor operator and the cognate promoter of the output promoter that is operably linked to the at least one output sequence are separated by 0 to 20 base pairs.

13.-14. (canceled)

15. The genetic circuit of claim 1, wherein the catalytically-inactive CRISPR/Cas protein and the transcription factor of the at least one fusion protein are separated by a linker peptide.

16. The genetic circuit of claim 1, wherein the single polynucleotide or the combination of polynucleotides encode:
(a) at least one fusion protein comprising a catalytically-inactive CRISPR/Cas protein fused to a transcription factor, wherein the catalytically-inactive CRISPR/Cas protein comprises a mutated PAM domain and a mutated or absent HNH domain;
(b) between two and thirty unique sgRNAs, wherein the expression of at least one of the unique sgRNAs is under the control of an inducible promoter; and
(c) between one and twenty-nine output sequences, each of whose expression is operably linked to an independent output promoter, wherein at least two of the output promoters comprise a transcription factor operator and a cognate promoter comprising a unique sgRNA target site and, optionally, a PAM site, and wherein:
(i) the unique sgRNA target site of each output promoter comprising an sgRNA target site comprises an sgRNA target site of one of the sgRNAs in (b); and
(ii) the unique sgRNA target site of at least one of the output promoters comprises the sgRNA target site of the at least one sgRNA under the control of an inducible promoter in (b).

17. The genetic circuit of claim 1, wherein
(a) the catalytically-inactive CRISPR/Cas protein of the at least one fusion protein consists of amino acids 1 to 1368 of Cas9, wherein the Cas9 amino acid sequence contains D10A and R1335K mutations and the Cas9 amino acids 768 to 919 are replaced by a GGSGGS (SEQ ID NO: 127) amino acid linker sequence,
(b) the transcription factor of the at least one fusion protein is PhlF,
(c) the catalytically-inactive CRISPR/Cas protein is fused to PhlF with a C-terminal polypeptide bond,
(d) the transcription factor operator of the output promoter that is operably linked to the at least one output sequence is a PhlF operator, and
(e) the PhlF operator and the cognate promoter of the output promoter that is operably linked to the at least one output sequence are separated by 0 to 20 base pairs.

18. The genetic circuit of claim 1, wherein the genetic circuit is encoded on a single polynucleotide, optionally wherein the single polynucleotide is a plasmid.

19. (canceled)

20. The genetic circuit of claim 1, wherein the genetic circuit is encoded on more than one polynucleotides, optionally wherein at least one of the polynucleotides is a plasmid.

21. (canceled)

22. A polynucleotide or combination of polynucleotides comprising the nucleotide sequence of the genetic circuit of claim 1.

23. (canceled)

24. A cell comprising the genetic circuit of the polynucleotide or combination of polynucleotides of claim 22.

25. A method of regulating expression of an output sequence of a genetic circuit comprising introducing the genetic circuit of claim 1 into a cell.

26. A fusion protein comprising a catalytically-inactive Cas9 protein linked by a C-terminal polypeptide bond to PhlF, wherein
(a) the catalytically-inactive Cas9 protein comprises a mutated PAM domain, a mutated HNH domain, and a functional RuvCI domain, and
(b) optionally, the catalytically-inactive Cas9 protein and the PhlF protein are separated by a linker peptide.

27. The fusion protein of claim 26, wherein the mutation of the Cas9 HNH domain consists of the deletion of the entire domain and its replacement by an amino acid linker sequence.

28. The fusion protein of claim 26, wherein the catalytically-inactive Cas9 protein amino acid sequence contains D10A and R1335K mutations and the Cas9 amino acids 768 to 919 are replaced by a GGSGGS (SEQ ID NO: 127) amino acid linker sequence.

29. A polynucleotide encoding for the fusion protein of claim 16.


SEQUENCE LISTING (139 sequences)

SEQ ID NO: 1 - DNA, 103 bp, Artificial Sequence
  Gate
ataatacccctactaggagtgttttagagctagaaatagcaagttaaaataaggctagtccgttatcaacttgaaaaagtggcaccgagtcggtgcttttttt

SEQ ID NO: 2 - DNA, 91 bp, Artificial Sequence
  Gate
tgtctacccgaaggcggcgtatgatacgaaacgtaccgtatcgttaaggtacatggtttacaccaactcctagtaggggtattatgctagc

SEQ ID NO: 3 - DNA, 103 bp, Artificial Sequence
  Gate
ataataccgcactctcctaggttttagagctagaaatagcaagttaaaataaggctagtccgttatcaacttgaaaaagtggcaccgagtcggtgcttttttt

SEQ ID NO: 4 - DNA, 91 bp, Artificial Sequence
  Gate
tttaggtttgccgacgcccgatgatacgaaacgtaccgtatcgttaaggtggtttgtttacaccactaggagagtgcggtattatgctagc

SEQ ID NO: 5 - DNA, 103 bp, Artificial Sequence
  Gate
ataataccctagggacccctgttttagagctagaaatagcaagttaaaataaggctagtccgttatcaacttgaaaaagtggcaccgagtcggtgcttttttt

SEQ ID NO: 6 - DNA, 91 bp, Artificial Sequence
  Gate
agacaaccttgacatggggcatgatacgaaacgtaccgtatcgttaaggtatacactttacaccaaggggtccctagggtattatgctagc

SEQ ID NO: 7 - DNA, 103 bp, Artificial Sequence
  Gate
ataataccagtcctagcctagttttagagctagaaatagcaagttaaaataaggctagtccgttatcaacttgaaaaagtggcaccgagtcggtgcttttttt

SEQ ID NO: 8 - DNA, 91 bp, Artificial Sequence
  Gate
aagtccttatctgcgcaatcatgatacgaaacgtaccgtatcgttaaggtcgatgatttacaccataggctaggactggtattatgctagc

SEQ ID NO: 9 - DNA, 103 bp, Artificial Sequence
  Gate
ataataccaggtcctaagtggttttagagctagaaatagcaagttaaaataaggctagtccgttatcaacttgaaaaagtggcaccgagtcggtgcttttttt

SEQ ID NO: 10 - DNA, 91 bp, Artificial Sequence
  Gate
tctatacatccgaagtcgagatgatacgaaacgtaccgtatcgttaaggttacagatttacaccacacttaggacctggtattatgctagc

SEQ ID NO: 11 - DNA, 103 bp, Artificial Sequence
  Gate
ataataccgaccctccctctgttttagagctagaaatagcaagttaaaataaggctagtccgttatcaacttgaaaaagtggcaccgagtcggtgcttttttt

SEQ ID NO: 12 - DNA, 91 bp, Artificial Sequence
  Gate
cacatcaatcgctaggtggcatgatacgaaacgtaccgtatcgttaaggtccggcgtttacaccaagagggagggtcggtattatgctagc

SEQ ID NO: 13 - DNA, 103 bp, Artificial Sequence
  Gate
ataatacctgtcctaacactgttttagagctagaaatagcaagttaaaataaggctagtccgttatcaacttgaaaaagtggcaccgagtcggtgcttttttt

SEQ ID NO: 14 - DNA, 91 bp, Artificial Sequence
  Gate
gattcgaataatctcgagccatgatacgaaacgtaccgtatcgttaaggttgctattttacaccaagtgttaggacaggtattatgctagc

SEQ ID NO: 15 - DNA, 103 bp, Artificial Sequence
  Gate
ataataccccctctagctaggttttagagctagaaatagcaagttaaaataaggctagtccgttatcaacttgaaaaagtggcaccgagtcggtgcttttttt

SEQ ID NO: 16 - DNA, 91 bp, Artificial Sequence
  Gate
gtctcgaacacctatcagttatgatacgaaacgtaccgtatcgttaaggtctcgactttacaccactagctagagggggtattatgctagc

SEQ ID NO: 17 - DNA, 103 bp, Artificial Sequence
  Gate
ataatacccgtgtgacccgtgttttagagctagaaatagcaagttaaaataaggctagtccgttatcaacttgaaaaagtggcaccgagtcggtgcttttttt

SEQ ID NO: 18 - DNA, 91 bp, Artificial Sequence
  Gate
tccgaatgacatgcgtctccatgatacgaaacgtaccgtatcgttaaggtacagcctttacaccaacgggtcacacgggtattatgctagc

SEQ ID NO: 19 - DNA, 103 bp, Artificial Sequence; identical to SEQ ID NO: 3
  Gate

SEQ ID NO: 20 - DNA, 91 bp, Artificial Sequence
  Gate
ccaaacgccatatctttgacatgatacgaaacgtaccgtatcgttaaggtacaacctttacaccactaggagagtgcggtattatgctagc

SEQ ID NO: 21 - DNA, 103 bp, Artificial Sequence
  Gate
ataataccctctagtctagagttttagagctagaaatagcaagttaaaataaggctagtccgttatcaacttgaaaaagtggcaccgagtcggtgcttttttt

SEQ ID NO: 22 - DNA, 91 bp, Artificial Sequence
  Gate
acaaagcctattacgatgacatgatacgaaacgtaccgtatcgttaaggttagtaatttacaccatctagactagagggtattatgctagc

SEQ ID NO: 23 - DNA, 103 bp, Artificial Sequence
  Gate
ataataccctcctagtctaggttttagagctagaaatagcaagttaaaataaggctagtccgttatcaacttgaaaaagtggcaccgagtcggtgcttttttt

SEQ ID NO: 24 - DNA, 91 bp, Artificial Sequence
  Gate
gtgagagtactttatacgctatgatacgaaacgtaccgtatcgttaaggttttgactttacaccactagactaggagggtattatgctagc

SEQ ID NO: 25 - DNA, 103 bp, Artificial Sequence
  Gate
ataataccactactagagtggttttagagctagaaatagcaagttaaaataaggctagtccgttatcaacttgaaaaagtggcaccgagtcggtgcttttttt

SEQ ID NO: 26 - DNA, 91 bp, Artificial Sequence
  Gate
tggtcgcagcagagcgaggaatgatacgaaacgtaccgtatcgttaaggtcgaagttttacaccacactctagtagtggtattatgctagc

SEQ ID NO: 27 - DNA, 103 bp, Artificial Sequence
  Gate
ataatacctgtcctagaggtgttttagagctagaaatagcaagttaaaataaggctagtccgttatcaacttgaaaaagtggcaccgagtcggtgcttttttt

SEQ ID NO: 28 - DNA, 91 bp, Artificial Sequence
  Gate
ccttgaggagctggttgtaaatgatacgaaacgtaccgtatcgttaaggtctgggctttacaccaacctctaggacaggtattatgctagc

SEQ ID NO: 29 - DNA, 103 bp, Artificial Sequence
  Gate
ataataccagtgtacctagtgttttagagctagaaatagcaagttaaaataaggctagtccgttatcaacttgaaaaagtggcaccgagtcggtgcttttttt

SEQ ID NO: 30 - DNA, 91 bp, Artificial Sequence
  Gate
tttctcagcgtaatcgttcgatgatacgaaacgtaccgtatcgttaaggtaccgaatttacaccaactaggtacactggtattatgctagc

SEQ ID NO: 31 - DNA, 103 bp, Artificial Sequence
  Gate
ataataccgacataggatctgttttagagctagaaatagcaagttaaaataaggctagtccgttatcaacttgaaaaagtggcaccgagtcggtgcttttttt

SEQ ID NO: 32 - DNA, 91 bp, Artificial Sequence
  Gate
cgagattcccttatccttttatgatacgaaacgtaccgtatcgttaaggtatgcactttacaccaagatcctatgtcggtattatgctagc

SEQ ID NO: 33 - DNA, 103 bp, Artificial Sequence
  Gate
ataataccgggagtcctatagttttagagctagaaatagcaagttaaaataaggctagtccgttatcaacttgaaaaagtggcaccgagtcggtgcttttttt

SEQ ID NO: 34 - DNA, 91 bp, Artificial Sequence
  Gate
gatcgcctcactttgaaattatgatacgaaacgtaccgtatcgttaaggtgcggcctttacaccatataggactcccggtattatgctagc

SEQ ID NO: 35 - DNA, 103 bp, Artificial Sequence; identical to SEQ ID NO: 5
  Gate

SEQ ID NO: 36 - DNA, 91 bp, Artificial Sequence
  Gate
aatagatacagttaggtttgatgatacgaaacgtaccgtatcgttaaggtgaccagtttacaccaaggggtccctagggtattatgctagc

SEQ ID NO: 37 - DNA, 103 bp, Artificial Sequence
  Gate
ataataccgtatgggactctgttttagagctagaaatagcaagttaaaataaggctagtccgttatcaacttgaaaaagtggcaccgagtcggtgcttttttt

SEQ ID NO: 38 - DNA, 91 bp, Artificial Sequence
  Gate
tgtagaggttaagcaggtcaatgatacgaaacgtaccgtatcgttaaggtcatgactttacaccaagagtcccatacggtattatgctagc

SEQ ID NO: 39 - DNA, 103 bp, Artificial Sequence
  Gate
ataataccagactctagggtgttttagagctagaaatagcaagttaaaataaggctagtccgttatcaacttgaaaaagtggcaccgagtcggtgcttttttt

SEQ ID NO: 40 - DNA, 91 bp, Artificial Sequence
  Gate
ttaaccactgtaagaaagttatgatacgaaacgtaccgtatcgttaaggtctcgtatttacaccaaccctagagtctggtattatgctagc

SEQ ID NO: 41 - DNA, 103 bp, Artificial Sequence
  Gate
ataatacctcctactagactgttttagagctagaaatagcaagttaaaataaggctagtccgttatcaacttgaaaaagtggcaccgagtcggtgcttttttt

SEQ ID NO: 42 - DNA, 91 bp, Artificial Sequence
  Gate
gccttgagttaggctctctcatgatacgaaacgtaccgtatcgttaaggtgcatattttacaccaagtctagtaggaggtattatgctagc

SEQ ID NO: 43 - DNA, 103 bp, Artificial Sequence
  Gate
ataatacctctagagtccctgttttagagctagaaatagcaagttaaaataaggctagtccgttatcaacttgaaaaagtggcaccgagtcggtgcttttttt

SEQ ID NO: 44 - DNA, 91 bp, Artificial Sequence
  Gate
ctccgtcggagttgacgtcgatgatacgaaacgtaccgtatcgttaaggttcggattttacaccaagggactctagaggtattatgctagc

SEQ ID NO: 45 - DNA, 103 bp, Artificial Sequence
  Gate
ataataccacccctagggacgttttagagctagaaatagcaagttaaaataaggctagtccgttatcaacttgaaaaagtggcaccgagtcggtgcttttttt

SEQ ID NO: 46 - DNA, 91 bp, Artificial Sequence
  Gate
acgactaccgcagtgcagtaatgatacgaaacgtaccgtatcgttaaggttttaattttacaccagtccctaggggtggtattatgctagc

SEQ ID NO: 47 - DNA, 103 bp, Artificial Sequence
  Gate
ataataccgacttggaccccgttttagagctagaaatagcaagttaaaataaggctagtccgttatcaacttgaaaaagtggcaccgagtcggtgcttttttt

SEQ ID NO: 48 - DNA, 91 bp, Artificial Sequence
  Gate
ccctgtcgttagtctccgagatgatacgaaacgtaccgtatcgttaaggttttaggtttacaccaggggtccaagtcggtattatgctagc

SEQ ID NO: 49 - DNA, 103 bp, Artificial Sequence
  Gate
ataataccaggacctagtatgttttagagctagaaatagcaagttaaaataaggctagtccgttatcaacttgaaaaagtggcaccgagtcggtgcttttttt

SEQ ID NO: 50 - DNA, 91 bp, Artificial Sequence
  Gate
ctgttccgcgtcacatcaacatgatacgaaacgtaccgtatcgttaaggtggagtatttacaccaatactaggtcctggtattatgctagc

SEQ ID NO: 51 - DNA, 103 bp, Artificial Sequence
  Gate
ataataccagtcctacctctgttttagagctagaaatagcaagttaaaataaggctagtccgttatcaacttgaaaaagtggcaccgagtcggtgcttttttt

SEQ ID NO: 52 - DNA, 91 bp, Artificial Sequence
  Gate
caactcgtgatatccgcctgatgatacgaaacgtaccgtatcgttaaggtgctcgctttacaccaagaggtaggactggtattatgctagc

SEQ ID NO: 53 - DNA, 103 bp, Artificial Sequence; identical to SEQ ID NO: 15
  Gate

SEQ ID NO: 54 - DNA, 91 bp, Artificial Sequence
  Gate
acggagtctgagacccggcgatgatacgaaacgtaccgtatcgttaaggtaagacctttacaccactagctagagggggtattatgctagc

SEQ ID NO: 55 - DNA, 103 bp, Artificial Sequence
  Gate
ataataccactagacctagtgttttagagctagaaatagcaagttaaaataaggctagtccgttatcaacttgaaaaagtggcaccgagtcggtgcttttttt

SEQ ID NO: 56 - DNA, 91 bp, Artificial Sequence
  Gate
ggttaagttgaacctccgatatgatacgaaacgtaccgtatcgttaaggtcacttctttacaccaactaggtctagtggtattatgctagc

SEQ ID NO: 57 - DNA, 103 bp, Artificial Sequence
  Gate
ataataccactagtccaagggttttagagctagaaatagcaagttaaaataaggctagtccgttatcaacttgaaaaagtggcaccgagtcggtgcttttttt

SEQ ID NO: 58 - DNA, 91 bp, Artificial Sequence
  Gate
ctcagaagctaccaatgtttatgatacgaaacgtaccgtatcgttaaggtttaaggtttacaccaccttggactagtggtattatgctagc

SEQ ID NO: 59 - DNA, 103 bp, Artificial Sequence
  Gate
ataataccgtctaggaccccgttttagagctagaaatagcaagttaaaataaggctagtccgttatcaacttgaaaaagtggcaccgagtcggtgcttttttt

SEQ ID NO: 60 - DNA, 91 bp, Artificial Sequence
  Gate
ggttctatatctctaggggtatgatacgaaacgtaccgtatcgttaaggtcgacattttacaccaggggtcctagacggtattatgctagc

SEQ ID NO: 61 - DNA, 82 bp, Artificial Sequence
  Promoter
tccgaatgacatgcgtctcccgctccaacaccgttggttgaacagcctttacaccaacgggtcacacgggtattatgctagc

SEQ ID NO: 62 - DNA, 82 bp, Artificial Sequence
  Promoter
tccgaatgacatgcgtctccggtgttggagcggttggttgaacagcctttacaccaacgggtcacacgggtattatgctagc

SEQ ID NO: 63 - DNA, 67 bp, Artificial Sequence
  Promoter
tccgaatgacatgcgtctccggtgttggagcgtttacaccaacgggtcacacgggtattatgctagc

SEQ ID NO: 64 - DNA, 69 bp, Artificial Sequence
  Promoter
tccgaatgacatgcgtctccggtgttggagcgcctttacaccaacgggtcacacgggtattatgctagc

SEQ ID NO: 65 - DNA, 71 bp, Artificial Sequence
  Promoter
tccgaatgacatgcgtctccggtgttggagcgagcctttacaccaacgggtcacacgggtattatgctagc

SEQ ID NO: 66 - DNA, 73 bp, Artificial Sequence
  Promoter
tccgaatgacatgcgtctccggtgttggagcgacagcctttacaccaacgggtcacacgggtattatgctagc

SEQ ID NO: 67 - DNA, 75 bp, Artificial Sequence
  Promoter
tccgaatgacatgcgtctccggtgttggagcggaacagcctttacaccaacgggtcacacgggtattatgctagc

SEQ ID NO: 68 - DNA, 87 bp, Artificial Sequence
  Promoter
tccgaatgacatgcgtctccatgatacgaaacgtaccgtatcgttaaggtcctttacaccaacgggtcacacgggtattatgctagc

SEQ ID NO: 69 - DNA, 89 bp, Artificial Sequence
  Promoter
tccgaatgacatgcgtctccatgatacgaaacgtaccgtatcgttaaggtagcctttacaccaacgggtcacacgggtattatgctagc

SEQ ID NO: 70 - DNA, 90 bp, Artificial Sequence
  Promoter
tccgaatgacatgcgtctccatgatacgaaacgtaccgtatcgttaaggtcagcctttacaccaacgggtcacacgggtattatgctagc

SEQ ID NO: 71 - DNA, 91 bp, Artificial Sequence; identical to SEQ ID NO: 18
  Promoter

SEQ ID NO: 72 - DNA, 92 bp, Artificial Sequence
  Promoter
tccgaatgacatgcgtctccatgatacgaaacgtaccgtatcgttaaggtaacagcctttacaccaacgggtcacacgggtattatgctagc

SEQ ID NO: 73 - DNA, 100 bp, Artificial Sequence
  Promoter
tccgaatgacatgcgtctccatgatacgaaacgtaccgtatcgttaaggtgttggttgaacagcctttacaccaacgggtcacacgggtattatgctagc

SEQ ID NO: 74 - DNA, 91 bp, Artificial Sequence
  Promoter
tccgaatgacatgcgtctccatatacatacatgcttgtttgtttgtaaacacagcctttacaccaacgggtcacacgggtattatgctagc

SEQ ID NO: 75 - DNA, 83 bp, Artificial Sequence
  Promoter
tccgaatgacatgcgtctccatatttaaaattcttgtttaaaacagcctttacaccaacgggtcacacgggtattatgctagc

SEQ ID NO: 76 - DNA, 81 bp, Artificial Sequence
  Promoter
tccgaatgacatgcgtctcccggaatgaacgttcattccgacagcctttacaccaacgggtcacacgggtattatgctagc

SEQ ID NO: 77 - DNA, 35 bp, Artificial Sequence
  Promoter
tttacagctagctcagtcctaggtattatgctagc

SEQ ID NO: 78 - DNA, 35 bp, Artificial Sequence
  Promoter
tttacaccaactcctagtaggggtattatgctagc

SEQ ID NO: 79 - DNA, 56 bp, Artificial Sequence
  Promoter
tgttgacaattaatcatcggctcgtataatgtgtggaattgtgagcgctcacaatt

SEQ ID NO: 80 - DNA, 50 bp, Artificial Sequence
  Promoter
attggatccaattgacagctagctcagtcctaggtaccattggatccaat

SEQ ID NO: 81 - DNA, 63 bp, Artificial Sequence
  Promoter
agcgcgggtgagagggattcgttaccaatagacaattgattggacgttcaatataatgctagc

SEQ ID NO: 82 - DNA, 55 bp, Artificial Sequence
  Promoter
acctgtaggatcgtacaggtttacgcaagaaaatggtttgttacagtcgaataaa

SEQ ID NO: 83 - DNA, 74 bp, Artificial Sequence
  Promoter
tactccaccgttggcttttttccctatcagtgatagagattgacatccctatcagtgatagagataatgagcac

SEQ ID NO: 84 - DNA, 195 bp, Artificial Sequence
  sgRNA array
tttacaccaactcctagtaggggtattatgctagcataataccgcactctcctaggttttagagctagaaatagcaagttaaaataaggctagtccgttatcaacttgaaaaagtggcaccgagtcggtgctttttttctcggtaccaaattttcgaaaaaagacgctgaaaagcgtcttttttcgttttggtcc

SEQ ID NO: 85 - DNA, 640 bp, Artificial Sequence
  sgRNA array
tttacaccaactcctagtaggggtattatgctagcataataccgcactctcctaggttttagagctagaaatagcaagttaaaataaggctagtccgttatcaacttgaaaaagtggcaccgagtcggtgctttttttctcggtaccaaattttcgaaaaaagacgctgaaaagcgtcttttttcgttttggtccccaaacgccatatctttgactccgttaacggtcacgagtttttacaccaactcctagtaggggtattatgctagcataatacctgtcctagaggtgttttagagctagaaatagcaagttaaaataaggctagtccgttatcaacttgaaaaagtggcaccgagtcggtgctttttttaaaaaaaaaaaaggcctcccaaatcggggggccttttttattgataacaaaaccttgaggagctggttgtaatttacaccaactcctagtaggggtattatgctagcataataccagtgtacctagtgttttagagctagaaatagcaagttaaaataaggctagtccgttatcaacttgaaaaagtggcaccgagtcggtgctttttttctcggtaccaaattccagaaaagagacgcttaacagcgtcttttttcgttttggtcc

SEQ ID NO: 86 - DNA, 1077 bp, Artificial Sequence
  sgRNA array
tttacaccaactcctagtaggggtattatgctagcataataccgcactctcctaggttttagagctagaaatagcaagttaaaataaggctagtccgttatcaacttgaaaaagtggcaccgagtcggtgctttttttctcggtaccaaattttcgaaaaaagacgctgaaaagcgtcttttttcgttttggtccccaaacgccatatctttgactccgttaacggtcacgagtttttacaccaactcctagtaggggtattatgctagcataatacctgtcctagaggtgttttagagctagaaatagcaagttaaaataaggctagtccgttatcaacttgaaaaagtggcaccgagtcggtgctttttttaaaaaaaaaaaaggcctcccaaatcggggggccttttttattgataacaaaaccttgaggagctggttgtaatttacaccaactcctagtaggggtattatgctagcataataccagtgtacctagtgttttagagctagaaatagcaagttaaaataaggctagtccgttatcaacttgaaaaagtggcaccgagtcggtgctttttttctcggtaccaaattccagaaaagagacgcttaacagcgtcttttttcgttttggtcctttctcagcgtaatcgttcgcgaaatcgaaggtgaaggtgtttacaccaactcctagtaggggtattatgctagcataataccgacataggatctgttttagagctagaaatagcaagttaaaataaggctagtccgttatcaacttgaaaaagtggcaccgagtcggtgctttttttccaattattgaaggccgctaacgcggcctttttttgtttctggtctccccgagattcccttatcctttttttacaccaactcctagtaggggtattatgctagcataataccgggagtcctatagttttagagctagaaatagcaagttaaaataaggctagtccgttatcaacttgaaaaagtggcaccgagtcggtgctttttttccaattattgaaggcctcccaaatcggggggccttttttattgataacaaaa

SEQ ID NO: 87 - DNA, 1292 bp, Artificial Sequence
  sgRNA array
tttacaccaactcctagtaggggtattatgctagcataatacctgtcctagaggtgttttagagctagaaatagcaagttaaaataaggctagtccgttatcaacttgaaaaagtggcaccgagtcggtgctttttttaaaaaaaaaaaaggcctcccaaatcggggggccttttttattgataacaaaaccttgaggagctggttgtaatttacaccaactcctagtaggggtattatgctagcataataccagtgtacctagtgttttagagctagaaatagcaagttaaaataaggctagtccgttatcaacttgaaaaagtggcaccgagtcggtgctttttttctcggtaccaaattccagaaaagagacgcttaacagcgtcttttttcgttttggtcctttctcagcgtaatcgttcgcgaaatcgaaggtgaaggtgtttacaccaactcctagtaggggtattatgctagcataataccgacataggatctgttttagagctagaaatagcaagttaaaataaggctagtccgttatcaacttgaaaaagtggcaccgagtcggtgctttttttccaattattgaaggccgctaacgcggcctttttttgtttctggtctccccgagattcccttatcctttttttacaccaactcctagtaggggtattatgctagcataataccgggagtcctatagttttagagctagaaatagcaagttaaaataaggctagtccgttatcaacttgaaaaagtggcaccgagtcggtgctttttttccaattattgaaggcctcccaaatcggggggccttttttattgataacaaaagatcgcctcactttgaaatttatcaaagagttcatgcgtttttacaccaactcctagtaggggtattatgctagcataataccctagggacccctgttttagagctagaaatagcaagttaaaataaggctagtccgttatcaacttgaaaaagtggcaccgagtcggtgctttttttctcggtaccaaaaaaaaaaaaaaagacgctgaaaagcgtcttttttcgttttggtccaatagatacagttaggtttgtttacaccaactcctagtaggggtattatgctagcataataccccctctagctaggttttagagctagaaatagcaagttaaaataaggctagtccgttatcaacttgaaaaagtggcaccgagtcggtgctttttttctcggtaccaaaaaaaaaaaaaaagacgctgaaaagcgtcttttttttttttggtcc

SEQ ID NO: 88 - DNA, 1743 bp, Artificial Sequence
  sgRNA array
tttacaccaactcctagtaggggtattatgctagcataatacctgtcctagaggtgttttagagctagaaatagcaagttaaaataaggctagtccgttatcaacttgaaaaagtggcaccgagtcggtgctttttttaaaaaaaaaaaaggcctcccaaatcggggggccttttttattgataacaaaaccttgaggagctggttgtaatttacaccaactcctagtaggggtattatgctagcataataccagtgtacctagtgttttagagctagaaatagcaagttaaaataaggctagtccgttatcaacttgaaaaagtggcaccgagtcggtgctttttttctcggtaccaaattccagaaaagagacgcttaacagcgtcttttttcgttttggtcctttctcagcgtaatcgttcgcgaaatcgaaggtgaaggtgtttacaccaactcctagtaggggtattatgctagcataataccgacataggatctgttttagagctagaaatagcaagttaaaataaggctagtccgttatcaacttgaaaaagtggcaccgagtcggtgctttttttccaattattgaaggccgctaacgcggcctttttttgtttctggtctccccgagattcccttatcctttttttacaccaactcctagtaggggtattatgctagcataataccgggagtcctatagttttagagctagaaatagcaagttaaaataaggctagtccgttatcaacttgaaaaagtggcaccgagtcggtgctttttttccaattattgaaggcctcccaaatcggggggccttttttattgataacaaaagatcgcctcactttgaaatttatcaaagagttcatgcgtttttacaccaactcctagtaggggtattatgctagcataataccctagggacccctgttttagagctagaaatagcaagttaaaataaggctagtccgttatcaacttgaaaaagtggcaccgagtcggtgctttttttctcggtaccaaaaaaaaaaaaaaagacgctgaaaagcgtcttttttcgttttggtccaatagatacagttaggtttgtttacaccaactcctagtaggggtattatgctagcataataccccctctagctaggttttagagctagaaatagcaagttaaaataaggctagtccgttatcaacttgaaaaagtggcaccgagtcggtgctttttttctcggtaccaaaaaaaaaaaaaaagacgctgaaaagcgtcttttttttttttggtccacggagtctgagactcggcgaaggtcgtccgtacgaaggttttacaccaactcctagtaggggtattatgctagcataataccgtatgggactctgttttagagctagaaatagcaagttaaaataaggctagtccgttatcaacttgaaaaagtggcaccgagtcggtgctttttttctcggtaccaaaccaattattgaagacgctgaaaagcgtcttttttcgttttggtcctgtagaggttaagcaggtcatttacaccaactcctagtaggggtattatgctagcataataccagactctagggtgttttagagctagaaatagcaagttaaaataaggctagtccgttatcaacttgaaaaagtggcaccgagtcggtgctttttttctcggtaccaaattccagaaaagagacgcttttagagcgtcttttttcgttttggtcc

SEQ ID NO: 89 - DNA, 2187 bp, Artificial Sequence
  sgRNA array
tttacaccaactcctagtaggggtattatgctagcataatacctgtcctagaggtgttttagagctagaaatagcaagttaaaataaggctagtccgttatcaacttgaaaaagtggcaccgagtcggtgctttttttaaaaaaaaaaaaggcctcccaaatcggggggccttttttattgataacaaaaccttgaggagctggttgtaatttacaccaactcctagtaggggtattatgctagcataataccagtgtacctagtgttttagagctagaaatagcaagttaaaataaggctagtccgttatcaacttgaaaaagtggcaccgagtcggtgctttttttctcggtaccaaattccagaaaagagacgcttaacagcgtcttttttcgttttggtcctttctcagcgtaatcgttcgcgaaatcgaaggtgaaggtgtttacaccaactcctagtaggggtattatgctagcataataccgacataggatctgttttagagctagaaatagcaagttaaaataaggctagtccgttatcaacttgaaaaagtggcaccgagtcggtgctttttttccaattattgaaggccgctaacgcggcctttttttgtttctggtctccccgagattcccttatcctttttttacaccaactcctagtaggggtattatgctagcataataccgggagtcctatagttttagagctagaaatagcaagttaaaataaggctagtccgttatcaacttgaaaaagtggcaccgagtcggtgctttttttccaattattgaaggcctcccaaatcggggggccttttttattgataacaaaagatcgcctcactttgaaatttatcaaagagttcatgcgtttttacaccaactcctagtaggggtattatgctagcataataccctagggacccctgttttagagctagaaatagcaagttaaaataaggctagtccgttatcaacttgaaaaagtggcaccgagtcggtgctttttttctcggtaccaaaaaaaaaaaaaaagacgctgaaaagcgtcttttttcgttttggtccaatagatacagttaggtttgtttacaccaactcctagtaggggtattatgctagcataataccccctctagctaggttttagagctagaaatagcaagttaaaataaggctagtccgttatcaacttgaaaaagtggcaccgagtcggtgctttttttctcggtaccaaaaaaaaaaaaaaagacgctgaaaagcgtcttttttttttttggtccacggagtctgagactcggcgaaggtcgtccgtacgaaggttttacaccaactcctagtaggggtattatgctagcataataccgtatgggactctgttttagagctagaaatagcaagttaaaataaggctagtccgttatcaacttgaaaaagtggcaccgagtcggtgctttttttctcggtaccaaaccaattattgaagacgctgaaaagcgtcttttttcgttttggtcctgtagaggttaagcaggtcatttacaccaactcctagtaggggtattatgctagcataataccagactctagggtgttttagagctagaaatagcaagttaaaataaggctagtccgttatcaacttgaaaaagtggcaccgagtcggtgctttttttctcggtaccaaattccagaaaagagacgcttttagagcgtcttttttcgttttggtccttaaccactgtaagaaagttacccagaccgctaaactgaatttacaccaactcctagtaggggtattatgctagcataatacctcctactagactgttttagagctagaaatagcaagttaaaataaggctagtccgttatcaacttgaaaaagtggcaccgagtcggtgctttttttctcggtaccaaattccagaaaagagacgctgaaaagcgtcttttttttttttggtccgccttgagttaggctctctctttacaccaactcctagtaggggtattatgctagcataatacctctagagtccctgttttagagctagaaatagcaagttaaaataaggctagtccgttatcaacttgaaaaagtggcaccgagtcggtgctttttttgacgaacaataaggcctccctaacggggggccttttttattgataacaaaa

SEQ ID NO: 90 - DNA, 2554 bp, Artificial Sequence
  sgRNA array
tttacaccaactcctagtaggggtattatgctagcataatacctgtcctagaggtgttttagagctagaaatagcaagttaaaataaggctagtccgttatcaacttgaaaaagtggcaccgagtcggtgctttttttaaaaaaaaaaaaggcctcccaaatcggggggccttttttattgataacaaaaccttgaggagctggttgtaatttacaccaactcctagtaggggtattatgctagcataataccagtgtacctagtgttttagagctagaaatagcaagttaaaataaggctagtccgttatcaacttgaaaaagtggcaccgagtcggtgctttttttctcggtaccaaattccagaaaagagacggtcgtccgtacgaaggttttacaccaactcctagtaggggtattatgctagcataataccgtatgggactctgttttagagctagaaatagcaagttaaaataaggctagtccgttatcaacttgaaaaagtggcaccgagtcggtgctttttttctcggtaccaaaccaattattgaagacgctgaaaagcgtcttttttcgttttggtcctgtagaggttaagcaggtcatttacaccaactcctagtaggggtattatgctagcataataccagactctagggtgttttagagctagaaatagcaagttaaaataaggctagtccgttatcaacttgaaaaagtggcaccgagtcggtgctttttttctcggtaccaaattccagaaaagagacgcttttagagcgtcttttttcgttttggtccttaaccactgtaagaaagttacccagaccgctaaactgaatttacaccaactcctagtaggggtattatgctagcataatacctcctactagactgttttagagctagaaatagcaagttaaaataaggctagtccgttatcaacttgaaaaagtggcaccgagtcggtgctttttttctcggtaccaaattccagaaaagagacgctgaaaagcgtcttttttttttttggtccgccttgagttaggctctctctttacaccaactcctagtaggggtattatgctagcataatacctctagagtccctgttttagagctagaaatagcaagttaaaataaggctagtccgttatcaacttgaaaaagtggcaccgagtcggtgctttttttgacgaacaataaggcctccctaacggggggccttttttattgataacaaaactccgtcggagttgacgtcgtgccgttcgcttgggacatctttacaccaactcctagtaggggtattatgctagcataataccaggacctagtatgttttagagctagaaatagcaagttaaaataaggctagtccgttatcaacttgaaaaagtggcaccgagtcggtgctttttttgacgaacaataaggcctcccgaaaggggggccttttttattgataacaaaactgttccgcgtcacatcaactttacaccaactcctagtaggggtattatgctagcataataccagtcctacctctgttttagagctagaaatagcaagttaaaataaggctagtccgttatcaacttgaaaaagtggcaccgagtcggtgcttttttttctaactaaaaacaccctaacgggtgtttttttgtttctggtctgcccaactcgtgatatccgcctgagttaccaaaggtggtccgctttacaccaactcctagtaggggtattatgctagcataataccacccctagggacgttttagagctagaaatagcaagttaaaataaggctagtccgttatcaacttgaaaaagtggcaccgagtcggtgctttttttccaattattgaacacccttcggggtgtttttttgtttctggtctcccacgactaccgcagtgcagtatttacaccaactcctagtaggggtattatgctagcataataccgacttggaccccgttttagagctagaaatagcaagttaaaataaggctagtccgttatcaacttgaaaaagtggcaccgagtcggtgctttttttccaattattgaagacgcttaacagcgtctttttttgtttctggtctccctcctgtcgttagtctccgagtcaaagttcgtatggaaggttttacaccaactcctagtaggggtattatgctagcataataccctcctagtctaggttttagagctagaaatagcaagttaaaataaggctagtccgttatcaacttgaaaaagtggcaccgagtcggtgctttttttttttcgaaaaaacaccctaacgggtgtttttttgtttctggtctcccgtgagagtactttatacgcttttacaccaactcctagtaggggtattatgctagcataataccactactagagtggttttagagctagaaatagcaagttaaaataaggctagtccgttatcaacttgaaaaagtggcaccgagtcggtgctttttttctcggtaccaaatctaactaaaaagacgctgaaaagcgtcttttttcgttttggtcc

SEQ ID NO: 91 - DNA, 3053 bp, Artificial Sequence
  sgRNA array
tttacaccaactcctagtaggggtattatgctagcataatacctgtcctagaggtgttttagagctagaaatagcaagttaaaataaggctagtccgttatcaacttgaaaaagtggcaccgagtcggtgctttttttaaaaaaaaaaaaggcctcccaaatcggggggccttttttattgataacaaaaccttgaggagctggttgtaatttacaccaactcctagtaggggtattatgctagcataataccagtgtacctagtgttttagagctagaaatagcaagttaaaataaggctagtccgttatcaacttgaaaaagtggcaccgagtcggtgctttttttctcggtaccaaattccagaaaagagacgcttaacagcgtcttttttcgttttggtcctttctcagcgtaatcgttcgcgaaatcgaaggtgaaggtgtttacaccaactcctagtaggggtattatgctagcataataccgacataggatctgttttagagctagaaatagcaagttaaaataaggctagtccgttatcaacttgaaaaagtggcaccgagtcggtgctttttttccaattattgaaggccgctaacgcggcctttttttgtttctggtctccccgagattcccttatcctttttttacaccaactcctagtaggggtattatgctagcataataccgggagtcctatagttttagagctagaaatagcaagttaaaataaggctagtccgttatcaacttgaaaaagtggcaccgagtcggtgctttttttccaattattgaaggcctcccaaatcggggggccttttttattgataacaaaagatcgcctcactttgaaatttatcaaagagttcatgcgtttttacaccaactcctagtaggggtattatgctagcataataccctagggacccctgttttagagctagaaatagcaagttaaaataaggctagtccgttatcaacttgaaaaagtggcaccgagtcggtgctttttttctcggtaccaaaaaaaaaaaaaaagacgctgaaaagcgtcttttttcgttttggtccaatagatacagttaggtttgtttacaccaactcctagtaggggtattatgctagcataataccccctctagctaggttttagagctagaaatagcaagttaaaataaggctagtccgttatcaacttgaaaaagtggcaccgagtcggtgctttttttctcggtaccaaaaaaaaaaaaaaagacgctgaaaagcgtcttttttttttttggtccacggagtctgagactcggcgaaggtcgtccgtacgaaggttttacaccaactcctagtaggggtattatgctagcataataccgtatgggactctgttttagagctagaaatagcaagttaaaataaggctagtccgttatcaacttgaaaaagtggcaccgagtcggtgctttttttctcggtaccaaaccaattattgaagacgctgaaaagcgtcttttttcgttttggtcctgtagaggttaagcaggtcatttacaccaactcctagtaggggtattatgctagcataataccagactctagggtgttttagagctagaaatagcaagttaaaataaggctagtccgttatcaacttgaaaaagtggcaccgagtcggtgctttttttctcggtaccaaattccagaaaagagacgcttttagagcgtcttttttcgttttggtccttaaccactgtaagaaagttacccagaccgctaaactgaatttacaccaactcctagtaggggtattatgctagcataatacctcctactagactgttttagagctagaaatagcaagttaaaataaggctagtccgttatcaacttgaaaaagtggcaccgagtcggtgctttttttctcggtaccaaattccagaaaagagacgctgaaaagcgtcttttttttttttggtccgccttgagttaggctctctctttacaccaactcctagtaggggtattatgctagcataatacctctagagtccctgttttagagctagaaatagcaagttaaaataaggctagtccgttatcaacttgaaaaagtggcaccgagtcggtgctttttttgacgaacaataaggcctccctaacggggggccttttttattgataacaaaactccgtcggagttgacgtcgtgccgttcgcttgggacatctttacaccaactcctagtaggggtattatgctagcataataccaggacctagtatgttttagagctagaaatagcaagttaaaataaggctagtccgttatcaacttgaaaaagtggcaccgagtcggtgctttttttgacgaacaataaggcctcccgaaaggggggccttttttattgataacaaaactgttccgcgtcacatcaactttacaccaactcctagtaggggtattatgctagcataataccagtcctacctctgttttagagctagaaatagcaagttaaaataaggctagtccgttatcaacttgaaaaagtggcaccgagtcggtgcttttttttctaactaaaaacaccctaacgggtgtttttttgtttctggtctgcccaactcgtgatatccgcctgagttaccaaaggtggtccgctttacaccaactcctagtaggggtattatgctagcataataccacccctagggacgttttagagctagaaatagcaagttaaaataaggctagtccgttatcaacttgaaaaagtggcaccgagtcggtgctttttttccaattattgaacacccttcggggtgtttttttgtttctggtctcccacgactaccgcagtgcagtatttacaccaactcctagtaggggtattatgctagcataataccgacttggaccccgttttagagctagaaatagcaagttaaaataaggctagtccgttatcaacttgaaaaagtggcaccgagtcggtgctttttttccaattattgaagacgcttaacagcgtctttttttgtttctggtctccc

SEQ ID NO: 92 - DNA, 3493 bp, Artificial Sequence
  sgRNA array
tttacaccaactcctagtaggggtattatgctagcataatacctgtcctagaggtgttttagagctagaaatagcaagttaaaataaggctagtccgttatcaacttgaaaaagtggcaccgagtcggtgctttttttaaaaaaaaaaaaggcctcccaaatcggggggccttttttattgataacaaaaccttgaggagctggttgtaatttacaccaactcctagtaggggtattatgctagcataataccagtgtacctagtgttttagagctagaaatagcaagttaaaataaggctagtccgttatcaacttgaaaaagtggcaccgagtcggtgctttttttctcggtaccaaattccagaaaagagacgcttaacagcgtcttttttcgttttggtcctttctcagcgtaatcgttcgcgaaatcgaaggtgaaggtgtttacaccaactcctagtaggggtattatgctagcataataccgacataggatctgttttagagctagaaatagcaagttaaaataaggctagtccgttatcaacttgaaaaagtggcaccgagtcggtgctttttttccaattattgaaggccgctaacgcggcctttttttgtttctggtctccccgagattcccttatcctttttttacaccaactcctagtaggggtattatgctagcataataccgggagtcctatagttttagagctagaaatagcaagttaaaataaggctagtccgttatcaacttgaaaaagtggcaccgagtcggtgctttttttccaattattgaaggcctcccaaatcggggggccttttttattgataacaaaagatcgcctcactttgaaatttatcaaagagttcatgcgtttttacaccaactcctagtaggggtattatgctagcataataccctagggacccctgttttagagctagaaatagcaagttaaaataaggctagtccgttatcaacttgaaaaagtggcaccgagtcggtgctttttttctcggtaccaaaaaaaaaaaaaaagacgctgaaaagcgtcttttttcgttttggtccaatagatacagttaggtttgtttacaccaactcctagtaggggtattatgctagcataataccccctctagctaggttttagagctagaaatagcaagttaaaataaggctagtccgttatcaacttgaaaaagtggcaccgagtcggtgctttttttctcggtaccaaaaaaaaaaaaaaagacgctgaaaagcgtcttttttttttttggtccacggagtctgagactcggcgaaggtcgtccgtacgaaggttttacaccaactcctagtaggggtattatgctagcataataccgtatgggactctgttttagagctagaaatagcaagttaaaataaggctagtccgttatcaacttgaaaaagtggcaccgagtcggtgctttttttctcggtaccaaaccaattattgaagacgctgaaaagcgtcttttttcgttttggtcctgtagaggttaagcaggtcatttacaccaactcctagtaggggtattatgctagcataataccagactctagggtgttttagagctagaaatagcaagttaaaataaggctagtccgttatcaacttgaaaaagtggcaccgagtcggtgctttttttctcggtaccaaattccagaaaagagacgcttttagagcgtcttttttcgttttggtccttaaccactgtaagaaagttacccagaccgctaaactgaatttacaccaactcctagtaggggtattatgctagcataatacctcctactagactgttttagagctagaaatagcaagttaaaataaggctagtccgttatcaacttgaaaaagtggcaccgagtcggtgctttttttctcggtaccaaattccagaaaagagacgctgaaaagcgtcttttttttttttggtccgccttgagttaggctctctctttacaccaactcctagtaggggtattatgctagcataatacctctagagtccctgttttagagctagaaatagcaagttaaaataaggctagtccgttatcaacttgaaaaagtggcaccgagtcggtgctttttttgacgaacaataaggcctccctaacggggggccttttttattgataacaaaactccgtcggagttgacgtcgtgccgttcgcttgggacatctttacaccaactcctagtaggggtattatgctagcataataccaggacctagtatgttttagagctagaaatagcaagttaaaataaggctagtccgttatcaacttgaaaaagtggcaccgagtcggtgctttttttgacgaacaataaggcctcccgaaaggggggccttttttattgataacaaaactgttccgcgtcacatcaactttacaccaactcctagtaggggtattatgctagcataataccagtcctacctctgttttagagctagaaatagcaagttaaaataaggctagtccgttatcaacttgaaaaagtggcaccgagtcggtgcttttttttctaactaaaaacaccctaacgggtgtttttttgtttctggtctgcccaactcgtgatatccgcctgagttaccaaaggtggtccgctttacaccaactcctagtaggggtattatgctagcataataccacccctagggacgttttagagctagaaatagcaagttaaaataaggctagtccgttatcaacttgaaaaagtggcaccgagtcggtgctttttttccaattattgaacacccttcggggtgtttttttgtttctggtctcccacgactaccgcagtgcagtatttacaccaactcctagtaggggtattatgctagcataataccgacttggaccccgttttagagctagaaatagcaagttaaaataaggctagtccgttatcaacttgaaaaagtggcaccgagtcggtgctttttttccaattattgaagacgcttaacagcgtctttttttgtttctggtctccctcctgtcgttagtctccgagtcaaagttcgtatggaaggttttacaccaactcctagtaggggtattatgctagcataataccctcctagtctaggttttagagctagaaatagcaagttaaaataaggctagtccgttatcaacttgaaaaagtggcaccgagtcggtgctttttttttttcgaaaaaacaccctaacgggtgtttttttgtttctggtctcccgtgagagtactttatacgcttttacaccaactcctagtaggggtattatgctagcataataccactactagagtggttttagagctagaaatagcaagttaaaataaggctagtccgttatcaacttgaaaaagtggcaccgagtcggtgctttttttctcggtaccaaatctaactaaaaagacgctgaaaagcgtcttttttcgttttggtcc

SEQ ID NO: 93 - DNA, 75 bp, Artificial Sequence
  insulator
agctgtcaccggatgtgctttccggtctgatgagtccgtgaggacgaaacagcctctacaaataattttgtttaa

SEQ ID NO: 94 - DNA, 4107 bp, Artificial Sequence
  gene
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 95 - DNA, 4635 bp, Artificial Sequence
  gene
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaaaagtatacgtctacaaaagaagttttagatgccactcttatccatcaatccatcactggtctttatgaaacacgcattgatttgagtcagctaggaggtgacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggaaagccctataaatgtcctgaatgtggcaagtccttctcgcggagcgacgacctgacacggcaccaacgtacgcacactggtgagaagccatacgcgtgtcctgtcgagtcctgtgaccgccgcttcagtcagaagggacacctgacacggcacatccgcattcacacagggcaaaaaccgtttcaatgccgcatctgcatgaggaacttcagcatccgtagcagcctgacacggcacatccgcacccacacaggagaaaagcccttcgcctgtgacatctgcggcaggaagttcgcgctgagccaccacctgacacggcacaccaagatccacctccgtcagaaagaccccgggtaa

SEQ ID NO: 96 - DNA, 4203 bp, Artificial Sequence
  gene
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagggaggttcaggtggatcgcgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaaaagtatacgtctacaaaagaagttttagatgccactcttatccatcaatccatcactggtctttatgaaacacgcattgatttgagtcagctaggaggtgacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggatttcgaatccggaaagccctataaatgtcctgaatgtggcaagtccttctcgcggagcgacgacctgacacggcaccaacgtacgcacactggtgagaagccatacgcgtgtcctgtcgagtcctgtgaccgccgcttcagtcagaagggacacctgacacggcacatccgcattcacacagggcaaaaaccgtttcaatgccgcatctgcatgaggaacttcagcatccgtagcagcctgacacggcacatccgcacccacacaggagaaaagcccttcgcctgtgacatctgcggcaggaagttcgcgctgagccaccacctgacacggcacaccaagatccacctccgtcagaaagaccccgggtaa

SEQ ID NO: 97 - DNA, 4293 bp, Artificial Sequence
  gene
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagggaggttcaggtggatcgcgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaaaagtatacgtctacaaaagaagttttagatgccactcttatccatcaatccatcactggtctttatgaaacacgcattgatttgagtcagctaggaggtgacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggattacccgtatgacgtacctgattacgctggttatccctatgatgtcccggactacgctggctcgtacccttatgatgtacctgactacgctttcgaatccggaaagccctataaatgtcctgaatgtggcaagtccttctcgcggagcgacgacctgacacggcaccaacgtacgcacactggtgagaagccatacgcgtgtcctgtcgagtcctgtgaccgccgcttcagtcagaagggacacctgacacggcacatccgcattcacacagggcaaaaaccgtttcaatgccgcatctgcatgaggaacttcagcatccgtagcagcctgacacggcacatccgcacccacacaggagaaaagcccttcgcctgtgacatctgcggcaggaagttcgcgctgagccaccacctgacacggcacaccaagatccacctccgtcagaaagaccccgggtaa

SEQ ID NO: 98 - DNA, 4536 bp, Artificial Sequence
  gene
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagggaggttcaggtggatcgcgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaaaagtatacgtctacaaaagaagttttagatgccactcttatccatcaatccatcactggtctttatgaaacacgcattgatttgagtcagctaggaggtgacggcaccggcgggcccaagaagaagaggaaggtatacccatacgatgttcctgactatgcgggctatccctatgacgtcccggactatgcaggatcgtatccttatgacgttccagattacgctggatccgccgctccggcagctaagaaaaagaaactggattacccgtatgacgtacctgattacgctggttatccctatgatgtcccggactacgctggctcgtacccttatgatgtacctgactacgctttcgaatccggagcacgtaccccgagccgtagcagcattggtagcctgcgtagtccgcatacccataaagcaattctgaccagcaccattgaaatcctgaaagaatgtggttatagcggtctgagcattgaaagcgttgcacgtcgtgccggtgcaagcaaaccgaccatttatcgttggtggaccaataaagcagcactgattgccgaagtgtatgaaaatgaaagcgaacaggtgcgtaaatttccggatctgggtagctttaaagccgatctggattttctgctgcgtaatctgtggaaagtttggcgtgaaaccatttgtggtgaagcatttcgttgtgttattgcagaagcacagctggaccctgcaaccctgacccagctgaaagatcagtttatggaacgtcgtcgtgagatgccgaaaaaactggttgaaaatgccattagcaatggtgaactgccgaaagataccaatcgtgaactgctgctggatatgatttttggtttttgttggtatcgcctgctgaccgaacagctgaccgttgaacaggatattgaagaatttaccttcctgctgattaatggtgtttgtccgggtacacagcgttaa

SEQ ID NO: 99 - DNA, 681 bp, Artificial Sequence
  gene
atggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataa

SEQ ID NO: 100 - DNA, 624 bp, Artificial Sequence
  gene
atgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcctaa

SEQ ID NO: 101 - DNA, 735 bp, Artificial Sequence
  gene
atggacatgcctcgtattaaaccgggtcagcgtgttatgatggcactgcgtaaaatgattgcaagcggtgaaatcaaaagtggtgaacgtattgcagaaattccgaccgcagcagcactgggtgttagccgtatgccggttcgtatcgcactgcgttcactggaacaagaaggtctggttgttcgtctgggtgcacgtggttatgcagcccgtggtgttagcagcgatcagattcgtgatgcaattgaagttcgtggtgttctggaaggttttgcagcacgtcgtctggcagaacgtggtatgaccgcagaaacccatgcacgttttgttgtactgattgcagaaggtgaagcactgtttgcagccggtcgcctgaatggtgaagatctggatcgttatgccgcatataatcaggcatttcatgataccctggttagcgcagcaggtaatggtgcagttgaaagcgcactggcacgtaatggttttgaaccgtttgcagcagccggtgcactggccctggatctgatggacctgtctgccgaatatgaacatctgctggcagcacatcgtcagcatcaggcagttctggatgcagttagctgtggtgatgccgaaggtgcagaacgtattatgcgtgatcatgcactggcagcaattcgtaatgcaaaagtttttgaagcagcagcaagcgcaggcgcaccgctgggtgcagcatggtcaattcgtgcagattga

SEQ ID NO: 102 - DNA, 588 bp, Artificial Sequence
  gene
atgccgaaactgggtatgcagagcattcgtcgtcgtcagctgattgatgcaaccctggaagcaattaatgaagttggtatgcatgatgcaaccattgcacagattgcacgtcgtgccggtgttagcaccggtattattagccattatttccgcgataaaaacggtctactggaagcaaccatgcgtgatattaccagccagctgcgtgatgcagttctgaatcgtctgcatgcactgccgcagggtagcgcagaacagcgtctgcaggcaattgttggtggtaattttgatgaaacccaggttagcagcgcagcaatgaaagcatggctggcattttgggcaatcagcatgcatcagccgatgctgtatcgtctgcagcaggttagcagtcgtcgtctgctgagcaatctggttagcgaatttcgtcgtgaactgcctcgtgaacaggcacaagaggcaggttatggtctggcagcactgattgatggtctgtggctgcgtgcagcactgagcggtaaaccgctggataaaacccgtgcaaatagcctgacccgtcattttatcacccagcatctgccgaccgattga

SEQ ID NO: 103 - DNA, 753 bp, Artificial Sequence
  gene
atgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggaatgcttagttttgcacattcagaaaaagacaactatatagatagtttatttttacatgcgtgtatgaacataccattaattgttccttctctagttgataattatcgaaaaataaatatagcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaa

SEQ ID NO: 104 - DNA, 52 bp, Artificial Sequence
  gene
aaaaaaaaaaaaggcctcccaaatcggggggccttttttattgataacaaaa

SEQ ID NO: 105 - DNA, 57 bp, Artificial Sequence
  Terminator
ctcggtaccaaattccagaaaagagacgcttaacagcgtcttttttcgttttggtcc

SEQ ID NO: 106 - DNA, 49 bp, Artificial Sequence
  Terminator
ccaattattgaaggccgctaacgcggcctttttttgtttctggtctccc

SEQ ID NO: 107 - DNA, 52 bp, Artificial Sequence
  Terminator
ccaattattgaaggcctcccaaatcggggggccttttttattgataacaaaa

SEQ ID NO: 108 - DNA, 57 bp, Artificial Sequence
  Terminator
ctcggtaccaaaaaaaaaaaaaaagacgctgaaaagcgtcttttttcgttttggtcc

SEQ ID NO: 109 - DNA, 57 bp, Artificial Sequence
  Terminator
ctcggtaccaaaaaaaaaaaaaaagacgctgaaaagcgtcttttttttttttggtcc

SEQ ID NO: 110 - DNA, 57 bp, Artificial Sequence
  Terminator
ctcggtaccaaaccaattattgaagacgctgaaaagcgtcttttttcgttttggtcc

SEQ ID NO: 111 - DNA, 58 bp, Artificial Sequence
  Terminator
ctcggtaccaaattccagaaaagagacgcttttagagcgtcttttttcgttttggtcc

SEQ ID NO: 112 - DNA, 57 bp, Artificial Sequence
  Terminator
ctcggtaccaaattccagaaaagagacgctgaaaagcgtcttttttttttttggtcc

SEQ ID NO: 113 - DNA, 51 bp, Artificial Sequence
  Terminator
gacgaacaataaggcctccctaacggggggccttttttattgataacaaaa

SEQ ID NO: 114 - DNA, 51 bp, Artificial Sequence
  Terminator
gacgaacaataaggcctcccgaaaggggggccttttttattgataacaaaa

SEQ ID NO: 115 - DNA, 47 bp, Artificial Sequence
  Terminator
tctaactaaaaacaccctaacgggtgtttttttgtttctggtctgcc

SEQ ID NO: 116 - DNA, 47 bp, Artificial Sequence
  Terminator
ccaattattgaacacccttcggggtgtttttttgtttctggtctccc

SEQ ID NO: 117 - DNA, 49 bp, Artificial Sequence
  Terminator
ccaattattgaagacgcttaacagcgtctttttttgtttctggtctccc

SEQ ID NO: 118 - DNA, 47 bp, Artificial Sequence
  Terminator
ttttcgaaaaaacaccctaacgggtgtttttttgtttctggtctccc

SEQ ID NO: 119 - DNA, 57 bp, Artificial Sequence
  Terminator
ctcggtaccaaatctaactaaaaagacgctgaaaagcgtcttttttcgttttggtcc

SEQ ID NO: 120 - DNA, 57 bp, Artificial Sequence
  Terminator
ctcggtaccaaagacgaacaataagacgctgaaaagcgtcttttttcgttttggtcc

SEQ ID NO: 121 - DNA, 57 bp, Artificial Sequence; identical to SEQ ID NO: 110
  Terminator

SEQ ID NO: 122 - DNA, 1083 bp, Artificial Sequence
  Terminator
atgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 123 - DNA, 57 bp, Artificial Sequence
  Terminator
ctcggtaccaaattccagaaaagagacgctttcgagcgtcttttttcgttttggtcc

SEQ ID NO: 124 - DNA, 57 bp, Artificial Sequence
  Terminator
ctcggtaccaaaccaattattgaagacgctgaaaagcgtctttttttgtttcggtcc

SEQ ID NO: 125 - DNA, 57 bp, Artificial Sequence
  Terminator
ggaaacacagaaaaaagcccgcacctgacagtgcgggctttttttttcgaccaaagg

SEQ ID NO: 126 - DNA, 129 bp, Artificial Sequence
  Terminator
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 127 - protein, 6 aa, Artificial Sequence
  Amino Acid Linker Sequence
GGSGGS

SEQ ID NO: 128 - protein, 1368 aa, Streptococcus pyogenes [sequence omitted]

SEQ ID NO: 129 - protein, 4 aa, Artificial Sequence
  Linker
GGGS

SEQ ID NO: 130 - protein, 6 aa, Artificial Sequence
  Linker
GGGGGG

SEQ ID NO: 131 - protein, 8 aa, Artificial Sequence
  Linker
GGGGGGGG

SEQ ID NO: 132 - protein, 15 aa, Artificial Sequence
  Linker
EAAAKEAAAKEAAAK

SEQ ID NO: 133 - protein, 46 aa, Artificial Sequence
  Linker
AEAAAKEAAAKEAAAKEAAAKALEAEAAAKEAAAKEAAAKEAAAKA

SEQ ID NO: 134 - protein, 5 aa, Artificial Sequence
  Linker
PAPAP

SEQ ID NO: 135 - protein, 17 aa, Artificial Sequence
  Linker
VSQTSKLTRAETVFPDV

SEQ ID NO: 136 - protein, 6 aa, Artificial Sequence
  Linker
RVLAEA

SEQ ID NO: 137 - protein, 10 aa, Artificial Sequence
  Linker
EDVVCCSMSY

SEQ ID NO: 138 - protein, 8 aa, Artificial Sequence
  Linker
GGIEGRGS

SEQ ID NO: 139 - protein, 4 aa, Artificial Sequence
  Linker
GFLG